(19) United States
(12) Patent Application Publication
(10) Pub. No.: US 2022/0049292 A1
(43) Pub. Date: Feb. 17, 2022

(54) MULTIPLEXED FLUORESCENT DETECTION OF ANALYTES

(71) Applicants: Illumina, Inc., San Diego, CA (US); IlLUMINA CAMBRIDGE LIMITED, Cambridge (GB)

(72) Inventors: Nikolai Romanov, Cambridge (GB); Stanley S. Hong, Palo Alto, CA (US); Patrick McCauley, Linton (GB); Xiaohai Liu, Cambridge (GB); Danilo Condello, San Francisco, CA (US); Merek Siu, San Diego, CA (US)

(21) Appl. No.: 17/250,836

(22) PCT Filed: Mar. 2, 2020
(86) PCT No.: PCT/EP2020/055426

§ 371 Date: Mar. 10, 2021

Related U.S. Application Data
(60) Provisional application No. 62/812,883, filed on Mar. 1, 2019.

(30) Foreign Application Priority Data
Jun. 17, 2019 (NL) 2023327

(51) Int. Cl. C12Q 1/6818; G01N 21/64

(52) U.S. Cl. CPC C12Q 1/6818; G01N 2021/6439; G01N 2021/6421; G01N 21/6428

(57) ABSTRACT

In a first aspect, a method includes: providing a sample, the sample including a first nucleotide and a second nucleotide; contacting the sample with a first fluorescent dye and a second fluorescent dye, the first fluorescent dye emitting first emitted light within a first wavelength band responsive to a first excitation illumination light, the second fluorescent dye emitting second emitted light within a second wavelength band responsive to a second excitation illumination light; simultaneously collecting, using one or more image detectors, multiplexed fluorescent light comprising the first emitted light and the second emitted light, the first emitted light being a first color channel corresponding to the first wavelength band and the second emitted light being a second color channel corresponding to the second wavelength band; and identifying the first nucleotide based on the first wavelength band of the first color channel and the second nucleotide based on the second wavelength band of the second color channel.

### CROSS-REFERENCE TO RELATED APPLICATIONS

This application claims the benefit of priority to U.S. Provisional Application No. 62/812,883, filed Mar. 1, 2019, and Dutch Application No. 2023327, filed Jun. 17, 2019. The entire contents of each of the aforementioned applications are hereby incorporated by reference.

### BACKGROUND

Sequencing by synthesis (SBS) technology uses modified deoxyribonucleotide triphosphates (dNTPs) including a terminator and a fluorescent dye having an emission spectrum. The fluorescent dye is covalently attached to a dNTP. The output of the fluorescent dye after irradiation by light (i.e., fluorescence) can be detected by a camera. When a single fluorescent color is used, each of the four bases are added in a separate cycle of DNA synthesis and imaging. In some implementations, separate fluorescent dyes for each of the four bases can be utilized. In further implementations, 2-channel and 4-channel SBS technology can use a mix of dye-labeled dNTPs. Images can be taken of each DNA cluster using light sources with different wavelength bands and output from appropriate fluorescent dyes with respective emission spectra

### SUMMARY

The present disclosure describes examples of systems or methods that can provide improved imaging throughput in an SBS system by simultaneously imaging a sample using two or more color channels. The dyes used and the characteristics of the color channels can facilitate low or no crosstalk between the color channels, such that the multiplexed fluorescent light can be used to identify nucleotides quickly, efficiently and reliably. This can provide significant improvements compared to other approaches that may require images to be captured sequentially, thereby providing a lower throughput. Any of multiple color channels can be used, including, but not limited to, blue and green color channels. Examples of systems or techniques that can be used to perform SBS based on multiplexed fluorescent light are described. Examples of dyes that can be used for labeling a sample to perform SBS based on multiplexed fluorescent light are described.

In a first aspect, a method includes providing a sample, the sample including a first nucleotide and a second nucleotide; contacting the sample with a first fluorescent dye and a second fluorescent dye, the first fluorescent dye emitting first emitted light within a first wavelength band responsive to a first excitation illumination light, the second fluorescent dye emitting second emitted light within a second wavelength band responsive to a second excitation illumination light; simultaneously collecting, using one or more image detectors, multiplexed fluorescent light comprising the first emitted light and the second emitted light, the first emitted light being a first color channel corresponding to the first wavelength band and the second emitted light being a second color channel corresponding to the second wavelength band; and identifying the first nucleotide based on the first wavelength band of the first color channel and the second nucleotide based on the second wavelength band of the second color channel.

Implementations can include any or all of the following features. The first wavelength band corresponds to a blue color and the second wavelength band corresponds to a green color. The first wavelength band is included within a range of about 450 nm to about 525 nm, and wherein the second wavelength band is included within a range of about 525 nm to about 650 nm. A first mean or peak wavelength is defined for a first emission spectrum of the first fluorescent dye, and a second mean or peak wavelength is defined for a second emission spectrum of the second fluorescent dye, the first and second mean or peak wavelengths having at least a predefined separation from each other. The first wavelength band has shorter wavelengths than the second wavelength band, wherein the second wavelength band is associated with a first wavelength, and wherein a wavelength emission separation between the first fluorescent dye and the second fluorescent dye is defined so that an emission spectrum of the first fluorescent dye includes at most a predefined amount of light at or above the first wavelength. Simultaneously collecting the multiplexed fluorescent light includes: detecting the first emitted light using a first optical subsystem for the first color channel, and detecting the second emitted light using a second optical subsystem for the second color channel, wherein an emission dichroic filter directs the first emitted light of the first color channel to the first optical subsystem and the second emitted light of the second color channel to the second optical subsystem. At least one of the first optical subsystem and the second optical subsystem includes an angled optical path. An emission spectrum of the first fluorescent dye has a peak in the first wavelength band. The sample further includes a third nucleotide, and the method further comprises: contacting the sample with a third fluorescent dye emitting third emitted light within the first wavelength band responsive to the first excitation illumination light, and emitting fourth emitted light within the second wavelength band responsive to the second excitation illumination, wherein the multiplexed fluorescent light further comprises the third emitted light and the fourth emitted light; and identifying the third nucleotide based on the first wavelength band of the first color channel and on the second wavelength band of the second color channel. The sample further includes a third nucleotide, and wherein the method further comprises: contacting the sample with a third fluorescent dye emitting third emitted light within a third wavelength band responsive to a third excitation illumination light, wherein the multiplexed fluorescent light further comprises the third emitted light; and identifying the third nucleotide based on the third wavelength band.

In a second aspect, an apparatus includes: a flow cell containing a sample, the sample including a first nucleotide and a second nucleotide, wherein the first nucleotide is coupled to a first fluorescent dye, wherein the second nucleotide is coupled to a second fluorescent dye, the first fluorescent dye emitting first emitted light within a first wavelength band responsive to a first excitation illumination light, the second fluorescent dye emitting second emitted light within a second wavelength band responsive to a second excitation illumination light; an illumination system simultaneously providing the first excitation illumination light and the second excitation illumination light to the flow cell; and a light collection system simultaneously collecting multiplexed fluorescent light comprising the first emitted light and the second emitted light, the first emitted light being a first color channel corresponding to the first wavelength band and the second emitted light being a second color channel corresponding to the second wavelength band.

Implementations can include any or all of the following features. The first wavelength band corresponds to a blue color and the second wavelength band corresponds to a green color. The first wavelength band is included within a range of about 450 nm to about 525 nm, and wherein the second wavelength band is included within a range of about 525 nm to about 650 nm. A first mean or peak wavelength is defined for a first emission spectrum of the first fluorescent dye, and a second mean or peak wavelength is defined for a second emission spectrum of the second fluorescent dye, the first and second mean or peak wavelengths having at least a predefined separation from each other. The first wavelength band has shorter wavelengths than the second wavelength band, wherein the second wavelength band is associated with a first wavelength, and wherein a wavelength emission separation between the first fluorescent dye and the second fluorescent dye is defined so that an emission spectrum of the first fluorescent dye includes at most a predefined amount of light at or above the first wavelength. The light collection system includes: a first optical subsystem for the first color channel detecting the first emitted light, and a second optical subsystem for the second color channel detecting the second emitted light, wherein an emission dichroic filter directs the first emitted light of the first color channel to the first optical subsystem and the second emitted light of the second color channel to the second optical subsystem. At least one of the first optical subsystem and the second optical subsystem includes an angled optical path. An emission spectrum of the first fluorescent dye has a peak in the first wavelength band. The sample further includes a third nucleotide coupled to a third fluorescent dye emitting third emitted light within the first wavelength band responsive to the first excitation illumination light, and emitting fourth emitted light within the second wavelength band responsive to the second excitation illumination, and wherein the multiplexed fluorescent light further comprises the third emitted light and the fourth emitted light. The sample further includes a third nucleotide coupled to a third fluorescent dye emitting third emitted light within a third wavelength band responsive to a third excitation illumination light, wherein the multiplexed fluorescent light further comprises the third emitted light.

The details of one or more examples of implementations are set forth in the accompanying drawings and the description below. Other features will be apparent from the description and drawings, and from the claims.

### BRIEF DESCRIPTION OF THE DRAWINGS

FIG. 1 is a diagram of a system including an instrument, a cartridge, and a flowcell.

FIG. 2 is a diagram of an illumination system including a flow cell according to an example implementation.

FIG. 3 is a diagram including plots of emission spectra of red and green dyes according to an example implementation.

FIG. 4 is a scatterplot illustrating a two-channel sequencing analysis having sequential imaging using green and red dyes of FIG. 3.

FIG. 5 is a scatter plot illustrating a two-channel sequencing analysis having simultaneous multiplexed imaging using green and red dyes of FIG. 3.

FIG. 6 is a diagram depicting metrics for the two-channel sequencing analyses of FIGS. 4-5.

FIG. 7 is a diagram including plots of emission spectra of blue and green dyes according to an example implementation.

FIG. 8 is a scatterplot illustrating a two-channel sequencing analysis having simultaneous multiplexed imaging using blue and green dyes of FIG. 7.

FIG. 9 is another diagram including plots of emission spectra of alternative blue and green dyes according to an example implementation.

FIG. 10 is a scatterplot illustrating a two-channel sequencing analysis having simultaneous multiplexed imaging using blue and green dyes of FIG. 9.

FIG. 11 is a scatterplot illustrating a two-channel sequencing analysis having simultaneous multiplexed imaging using other blue and green dyes.

FIG. 12 is a scatterplot illustrating a two-channel sequencing analysis having simultaneous multiplexed imaging using still other blue and green dyes.

FIG. 13 is another diagram including plots of emission spectra of alternative blue and green dyes and corresponding filter ranges according to an example implementation.

FIG. 14 is a scatterplot illustrating a two-channel sequencing analysis having simultaneous multiplexed imaging using blue and green dyes of FIG. 13 using a first filter range.

FIG. 15 is a scatterplot illustrating a two-channel sequencing analysis having simultaneous multiplexed imaging using blue and green dyes of FIG. 13 using a second filter range.

FIG. 16 is a scatterplot illustrating a two-channel sequencing analysis having simultaneous multiplexed imaging using the blue and green dyes of FIG. 9 and the second filter rage of FIG. 13.

FIG. 17 is a diagram depicting metrics for the two-channel sequencing analysis of FIG. 16.

FIG. 18 is a diagram representing a timeline of example sequential steps that may be involved in producing and analyzing multiplexed fluorescence images.

FIG. 19 is a diagram representing another timeline of example sequential steps that may be involved in producing and analyzing multiplexed fluorescence images.

FIG. 20 is a diagram representing a timeline of events involved in producing simultaneous images utilizing SIM imaging.

FIG. 21 is a flow chart illustrating a method of simultaneously imaging a sample according to an example implementation.

FIG. 22 is a flow chart illustrating a method of performing a sequencing operation.

FIG. 23 is another flow chart illustrating a method of performing a sequencing operation.

FIG. 24 is a scatterplot illustrating the usability of a fully functionalized A nucleotide labeled with dye I-4 described herein in a two-channel sequencing analysis.

FIG. 25 is a scatterplot illustrating the usability of a fully functionalized A nucleotide labeled with dye I-5 described herein in a two-channel sequencing analysis.

FIG. 26 is a scatterplot illustrating the usability of a fully functionalized A nucleotide labeled with dye I-6 described herein in a two-channel sequencing analysis.

### DETAILED DESCRIPTION

This document describes examples of systems and techniques that can provide robust sequencing by synthesis (SBS) results using simultaneous imaging of DNA clusters using two or more color channels. Such systems/techniques can provide one or more advantages over existing approaches, for example as described herein.

### I. Overview

Some approaches to performing SBS involve imaging of each wavelength band of emitted light from a corresponding fluorescent dye sequentially. That is, imaging a first wavelength band of emitted light corresponding to a first nucleotide, imaging a second wavelength band of emitted light corresponding to a second nucleotide, imaging a third wavelength band of emitted light corresponding to a third nucleotide, and imaging a fourth wavelength band of emitted light corresponding to a fourth nucleotide.

In some instances, such a sequential imaging process may lead to a low throughput of data due to separately imaging the four different wavelength bands of emitted light. In some implementations, two wavelength bands of emitted light have been utilized for identifying each nucleotide by reducing the number of images to deduce the nucleotide type to two, such as two-channel sequencing by synthesis. For example, a first wavelength band can be associated with two nucleotides, such as adenine and thymine. A second wavelength band can be associated with one overlapping nucleotide and a third nucleotide, such as adenine and cytosine.

The wavelength bands can be accomplished via fluorescent dyes that emit light within the corresponding wavelength band responsive to excitation light. In some implementations, two dyes, one for the first wavelength band and one for the second wavelength band, can each couple to a corresponding portion of a nucleic acid segment for adenine. Given a population of nucleic acid segments generated through amplification, at least some portions of a cluster of the population of nucleic acid segments can couple to the dye for the first wavelength band and to the dye for the second wavelength band. Thus, when the first dye is exposed to a first excitation light, the cluster emits light in the first wavelength band. When the second dye is exposed to a second excitation light, different from the first excitation light, the cluster emits light in the second wavelength band. Similarly, a dye emitting in the first wavelength band can couple to a corresponding portion of a nucleic acid segment for thymine and a dye emitting in the second wavelength band can couple to a corresponding portion of a nucleic acid segment for cytosine.

When the first wavelength band is imaged using a corresponding excitation light, an image for the first wavelength band emitted light can be acquired. When the second wavelength band is imaged using a corresponding excitation light, an image for the second wavelength band emitted light can be acquired. The acquisition of these images is temporally spaced such that the image acquisition of the first wavelength band of emitted light does not overlap with the second wavelength band of emitted light.

Portions of the two images that are depicted in both images can be determined to correspond to the overlapping nucleotide, such as adenine. Portions of the two images that are depicted in only the first image (and not emitting light in the second image) can be determined to correspond to the non-overlapping nucleotide associated with the first wavelength band emitting dye, such as thymine. Portions of the two images that are depicted in only the second image (and not emitting light in the first image) can be determined to correspond to the non-overlapping nucleotide associated with the second wavelength band emitting dye, such as cytosine. Portions of the two images that do not emit light in either the first or second first image can be determined to correspond to the fourth nucleotide, such as guanine.

In the foregoing systems, two or more sequential (i.e., temporally spaced) images are utilized to determine corresponding nucleotides. As described herein, simultaneous capturing of two or more different wavelength bands of emitted light may be achieved during a single imaging step, thereby eliminating the temporally spaced second set of images and thereby improving sequencing throughput by reducing the imaging steps to a single sequence for imaging. However, there may be difficulties to accomplishing the simultaneous two or more channel emitted light acquisition due to overlapping wavelength bands of emitted light. For example, in some cases when emitted light wavelength bands are too close to each other (e.g., blue and green bands), the respective emission spectra of different fluorescent dyes may overlap. In such cases, spectral “crosstalk” can occur and result in difficulties in processing to determine a corresponding nucleotide.

Described herein are systems and methods that simultaneously capture two or more wavelengths of emitted light in a single imaging step that can then be processed to determine corresponding nucleotides for a sequencing by synthesis process. In particular, a first wavelength band can be associated with two nucleotides, such as adenine and thymine. A second wavelength band can be associated with one nucleotide overlapping with the two nucleotides, and a third nucleotide, such as adenine and cytosine. The first wavelength band can have a first lower wavelength and a first upper wavelength. The second wavelength band can have a second lower wavelength and a second upper wavelength. In some implementations, the first lower wavelength is at least 50 nm from the second upper wavelength. In some implementations, the first lower wavelength and the second upper wavelength are set such that crosstalk is below a first predetermined value, such as 20%.

Separation between dyes can be defined in one or more other ways. This can be based on one or more of a wavelength band, color channel, or a fluorescent dye. A wavelength band can include all frequencies (e.g., an essentially continuous range of frequencies) between a first wavelength and a second wavelength. For example, the first and second wavelengths can be chosen so that the wavelength band includes blue light, or light of another color. A color channel represents the frequency or frequencies that are being detected by a detector. For example, frequencies of emitted light that are not within the color channel can be filtered out before reaching the detector. In some implementations, a color channel can include one or more wavelength bands. A fluorescent dye can be characterized in multiple ways, including, but not limited to, by its chemical structure and/or by its optical properties. In some implementations, a fluorescent dye can be characterized as emitting fluorescent light only in one or more wavelength bands, or as having a mean or peak wavelength at a frequency or within a wavelength band.

In some implementations, the separation can be defined based on an amount of emitted light from a corresponding dye above or below a predefined wavelength. The separation can be defined such that the amount of emitted light is at most a predetermined percentage above or below the predefined wavelength associated with the wavelength band of the other dye. For example, at most X percent of the fluorescent light of the dye is emitted above or below the other dye's predefined wavelength. In some implementations, the number X in the preceding example can be any suitable number, such as a range of values. For example, the range can be about 0-10% of the fluorescent light. As another example, the range can be about 0.5-5% of the fluorescent light. As another example, the range can be about 0.1-1% of the fluorescent light. In some implementations, a mean or peak wavelength separation between dyes can be used. For example, two dyes can be deemed to satisfy a separation metric if their mean or peak wavelengths are separated by at least a predetermined measure (e.g., a distance, or a percentage of either wavelength).

One or more fluorescent dyes can be utilized to emit light within the aforementioned wavelength bands. For instance, some dyes described herein may have an emission spectrum localized in a blue wavelength band to emit light for a blue color channel. Similarly, some dyes described herein may have an emission spectrum localized in a green wavelength band to emit light for a green color channel. Similarly, some dyes described herein may have an emission spectrum localized in a red wavelength band to emit light for a red color channel. For example, blue and green color channels can be detected. As another example, blue, green and red color channels can be detected. The emission spectrum of the dyes can be selected such that each is sufficiently localized in a blue spectral region and green spectral region, respectively, so as to have a reduced emission wavelength overlap

### II. Example Instrument and Illumination System for Multiplexed Fluorescence Detection

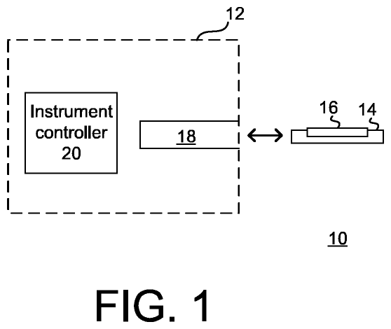

FIG. 1 is a diagram of a system 10 including an instrument 12, a cartridge 14, and a flowcell 16. The system 10 can be used for biological and/or chemical analysis. The system 10 can be used together with, or in the implementation of, one or more other examples described elsewhere herein.

The cartridge 14 can serve as a carrier for one or more samples, such as by way of the flowcell 16. The cartridge 14 can be configured to hold the flowcell 16 and transport the flowcell 16 into and out of direct interaction with the instrument 12. For example, the instrument 12 includes a receptacle 18 (e.g., an opening in its outer enclosure) to receive and accommodate the cartridge 14 at least during gathering of information from the sample. The cartridge 14 can be made of any suitable material(s). In some implementations, the cartridge 14 includes molded plastic or other durable material. For example, the cartridge 14 can form a frame for supporting or holding the flowcell 16.

Examples herein mention samples that are being analyzed. Such samples may include genetic material. In some implementations, the sample includes one or more template strands of genetic material. For example, using techniques and/or systems described herein, SBS can be performed on one or more template DNA strands.

The flowcell 16 can include one or more substrates configured for holding the sample(s) to be analyzed by the instrument 12. Any suitable material can be used for the substrate, including, but not limited to, glass, acrylic, and/or another plastic material. The flowcell 14 can allow liquids or other fluids to selectively be flowed relative to the sample(s). In some implementations, the flowcell 16 includes one or more flow structures that can hold the sample(s). In some implementations, the flowcell 12 can include at least one flow channel. For example, a flow channel can include one or more fluidic ports to facilitate flow of fluid.

The instrument 12 can operate to obtain any information or data that relates to at least one biological and/or chemical substance. The operation(s) can be controlled by a central unit or by one or more distributed controllers. Here, an instrument controller 20 is illustrated. For example, the controller 20 can be implemented using at least one processor, at least one storage medium (e.g., a memory and/or a drive) holding instructions for the operations of the instrument 12, and one or more other components, for example as described in the following. In some implementations, the instrument 12 can perform optical operations, including, but not limited to, illumination and/or imaging of the sample(s). For example, the instrument 12 can include one or more optical subsystems (e.g., an illumination subsystem and/or an imaging subsystem). In some implementations, the instrument 12 can perform thermal treatment, including, but not limited to, thermal conditioning of the sample(s). For example, the instrument 12 can include one or more thermal subsystems (e.g., a heater and/or cooler). In some implementations, the instrument 12 can perform fluid management, including, but not limited to, adding and/or removing fluid in contact with the sample(s). For example, the instrument 12 can include one or more fluid subsystems (e.g., a pump and/or a reservoir).

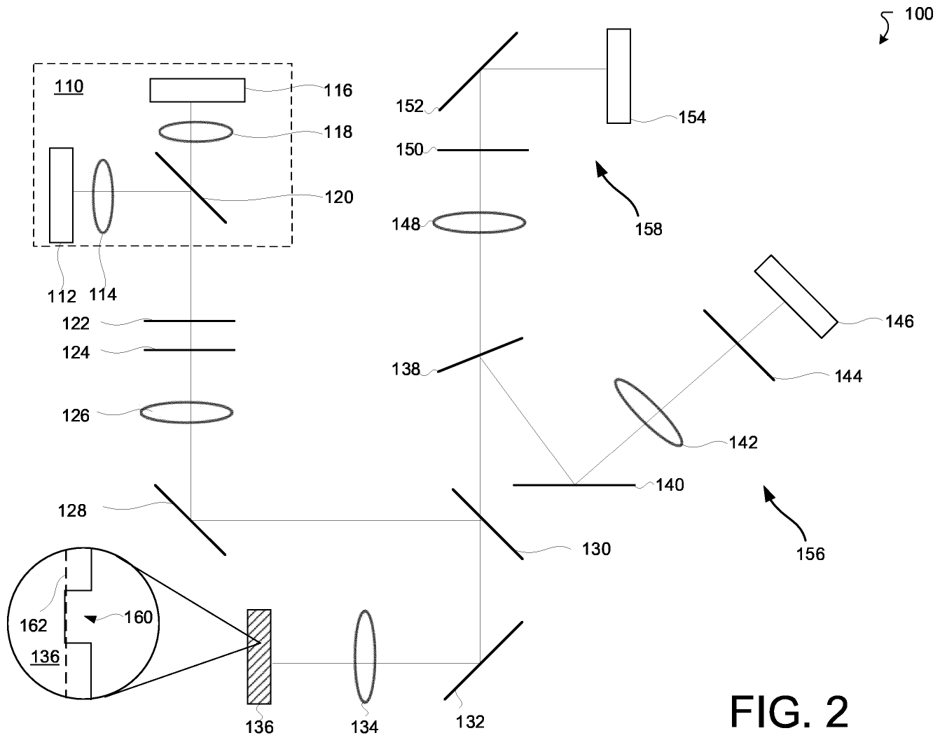

FIG. 2 is a diagram of an example illumination system 100. The illumination system 100 includes a light source assembly 110, an excitation dichroic filter 128, an objective lens 134, a flowcell 136, an emission dichroic filter 138, a first optical detection subsystem 156, and a second optical detection subsystem 158. The illumination system 100 enables simultaneous imaging of two color channels. In some implementations, another illumination system can be configured to enable simultaneous imaging of more than two color channels, e.g., three color channels, four color channels, or more. It is noted that there may be other optical configurations that can produce a similar, simultaneous imaging of multiple color channels.

The light source assembly 110 produces excitation illumination that is incident on the flowcell 136. This excitation illumination in turn will produce emitted illumination, or fluoresced illumination, from one or more fluorescent dyes that will be collected using the projection lenses 142 and 148. As shown in FIG. 2, the light source assembly 110 includes a first excitation illumination source 112 and corresponding converging lens 114, a second excitation illumination source 116 and corresponding converging lens 118, and a dichroic filter 120.

The first excitation illumination source 112 and the second excitation illumination source 116 exemplify an illumination system that can simultaneously provide respective excitation illumination lights for a sample (e.g., corresponding to respective color channels). In some implementations, each of the first excitation illumination source 112 and the second excitation illumination source 116 includes a light emitting diode (LED). In some implementations, at least one of the first excitation illumination source 112 and the second excitation illumination source 116 includes a laser. In some implementations, the first excitation illumination source 112 produces green light, i.e., narrow-band light with a peak or mean wavelength corresponding to a green color (e.g., about 560 nm). In some implementations, the second excitation illumination source 116 produces blue light, i.e., narrow-band light with a peak or mean wavelength corresponding to a blue color (e.g., about 490 nm).

The converging lenses 114 and 118 are each set a distance from the respective excitation illumination sources 112 and 116 such that the illumination emerging from each of the converging lenses 114/118 is focused at a field aperture 122.

The dichroic filter 120 reflects illumination from the first excitation illumination source 112 and transmits illumination from the second excitation illumination source 116. In some implementations, where the first excitation illumination source 112 produces green light and the second excitation illumination source 116 produces blue light, the dichroic filter reflects green light and transmits blue light. The dichroic filter 120 outputs mixed illumination with a mix of the two wavelengths, blue and green in the present example, onward through the optical path to be emitted by the objective lens 134.

In some implementations, the mixed excitation illumination output from the dichroic filter 120 can directly propagate toward the objective lens 134. In other implementations, the mixed excitation illumination can be further modified and/or controlled by additional intervening optical components prior to emission from the objective lens 134. In the example shown in FIG. 1, the mixed excitation illumination passes through a focus in the field aperture 122 to a blue/green filter 124 and then to a color-corrected collimating lens 126. The collimated excitation illumination from the lens 126 is incident upon a mirror 128 upon which it reflects and is incident on an excitation/emission dichroic filter 130. The excitation/emission dichroic filter 130 reflects the excitation illumination emitted from the light source assembly 110 while permitting emission illumination, which will be described further below, to pass through the excitation/emission dichroic filter 130 to be received by one or more optical subsystems 156, 158. The optical subsystems 156 and 158 exemplify a light collection system that can simultaneously collect multiplexed fluorescent light. The excitation illumination reflected from the excitation/emission dichroic filter 130 is then incident upon a mirror 132, from which it is incident upon the objective lens 134 towards the flowcell 136.

The objective lens 134 focuses the collimated excitation illumination from the mirror 132 onto the flowcell 136. In some implementations, the objective lens 134 is a microscope objective with a specified magnification factor of, for example, 1×, 2×, 4×, 5×, 6×, 8×, 10×, or higher. The objective lens 134 focuses the excitation illumination incident from the mirror 132 onto the flowcell 136 in a cone of angles, or numerical aperture, determined by the magnification factor. In some implementations, the objective lens 134 is movable on an axis that is normal to the flow cell (a “z-axis”). In some implementations, the illumination system 100 adjusts the z position of the tube lens 148 and tube lens 142 independently. For example, this can bring the green channel into focus on detector 154 and the blue channel perfectly into focus on detector 146 without having to move in z the objective. The independent adjustments in z of the tube lenses 148 and 142 may be a “one time adjustment” done when aligning the instrument for the first time.

The flowcell 136 contains a sample, such as a nucleotide sequence, to be analyzed. The flowcell 136 can include one or more channels 160 (here schematically illustrated by way of a cross-section view in an enlargement) configured to hold sample material and to facilitate actions to be taken with regard to the sample material, including, but not limited to, triggering chemical reactions or adding or removing material. An object plane 162 of the objective lens 134, here schematically illustrated using a dashed line, extends through the flowcell 136. For example, the object plane 162 can be defined so as to be adjacent the channel(s) 160.

The objective lens 134 can define a field of view. The field of view can define the area on the flowcell 136 from which an image detector captures emitted light using the objective lens 134. One or more image detectors, e.g., detectors 146 and 154, can be used. For example, when the first and second excitation illumination sources 112 and 116 generate respective excitation illumination having different wavelengths (or different wavelength ranges), the illumination system 100 can include separate image detectors 146 and 154 for the respective wavelengths (or wavelength ranges) of the emitted light. At least one of the image detectors 146 and 154 can include a charge-coupled device (CCD), such as a time-delay integration CCD camera, or a sensor fabricated based on complementary metal-oxide-semiconductor (CMOS) technology, such as chemically sensitive field effect transistors (chemFET), ion-sensitive field effect transistors (ISFET), and/or metal oxide semiconductor field effect transistors (MOSFET).

In some implementations, the illumination system 100 can include a structured illumination microscope (SIM). SIM imaging is based on spatially structured illumination light and reconstruction to result in a higher resolution image than an image produced solely using the magnification from the objective lens 134. For example, the structure can consist of or include a pattern or grating that interrupts the illuminating excitation light. In some implementations, the structure can include patterns of fringes. Fringes of light can be generated by impinging a light beam on a diffraction grating such that reflective or transmissive diffraction occurs. The structured light can be projected onto the sample, illuminating the sample according to the respective fringes which may occur according to some periodicity. To reconstruct an image using SIM, the two or more patterned images are used where the pattern of excitation illumination are at different phase angles to each other. For example, images of the sample can be acquired at different phases of the fringes in the structured light, sometimes referred to as the respective pattern phases of the images. This can allow various locations on the sample to be exposed to a multitude of illumination intensities. The set of resulting emitted light images can be combined to reconstruct the higher resolution image.

The sample material in the flowcell 136 is contacted with fluorescent dyes that couple to corresponding nucleotides. The fluorescent dyes emit fluorescent illumination upon being irradiated with corresponding excitation illumination incident on the flowcell 136 from the objective lens 134. The emitted illumination is identified with wavelength bands, each of which is can be categorized to a respective color channel. For example, the wavelength bands of the emitted illumination can correspond to a blue color (e.g., 450 nm-525 nm), a green color (e.g., 525 nm-570 nm), a yellow color (e.g., 570 nm-625 nm), a red color (e.g., 625 nm-750 nm), etc. In some implementations, the wavelength bands may be defined based on the two or more light wavelengths present during the simultaneous illumination. For example, when only blue and green colors are to be analyzed, the wavelength band corresponding to blue and green colors can be defined as different wavelength bands than the aforementioned ranges. For instance, a blue wavelength band can be set as emitted light from about 450 nm to 510 nm, such as 486 nm-506 nm. In some instances, the blue wavelength band can simply have an upper limit, such as about 500 nm-510 nm or about 506 nm. Similarly, the green wavelength band can be set as emitted light from about 525 nm to 650 nm, such as 584 nm-637 nm. While the foregoing green wavelength band extends into the yellow and red colors noted above, when analyzing emitted light expected to be in only the blue and green color ranges, the upper and/or lower ends of the wavelength band can be extended to capture additional emitted light that is emitted above or below the wavelength for the color. In some instances, the green wavelength band can simply have a lower limit, such as about 550 nm-600 nm or about 584 nm.

The fluorescent dyes are chemically conjoined with respective nucleotides, e.g., containing respective nucleobases. In this way, a dNTP labeled with a fluorescent dye may be identified based upon an emitted light wavelength being within a corresponding wavelength band when detected by an image detector 146, 154. That is, a first dNTP labeled with a blue dye can be identified responsive to an image detector 146, 154 receiving emitted light within a defined blue wavelength band, as discussed above. Similarly, another nucleotide labeled with a green dye may be identified responsive to an image detector 146, 154 receiving emitted light within a defined green wavelength band, as discussed above. Other color combinations of dye-labeled nucleotides for simultaneous DNA cluster imaging can also be used for sequencing with conjunction with appropriate illumination light sources and optical setup (e.g., blue and yellow; blue and red; green and red; yellow and red; blue, green, and red; blue, green, and yellow; blue, yellow, and red; green, yellow, and red; blue, green, yellow, and red; etc.).

The makeup of the fluorescent dyes is discussed in further detail below in section III describing various dyes. In some implementations, the fluorescent dyes are constructed such that each nucleotide may be robustly identified with a color channel using the simultaneous imaging platform enabled by the illumination system 100. Through selection of dye emission spectrum and filtering, multiplexed emitted light from the dyes can be implemented. In particular, because wavelength bands for near or similar colors, such as blue and green color channels, can be relatively close together, selection of certain fluorescent dyes with corresponding emission spectra that have a sufficiently small overlap can assist in reducing potential misidentification of nucleotides and, accordingly, errors in sequencing. In addition, the usage of waveband filtering can further aid in distinguishing certain fluorescent dyes that may have similar colors.

In some implementations, four types of nucleotides may be identified using two color channels. In that case, a first nucleotide may be associated with the first color channel only, a second nucleotide may be associated with the second color channel only, a third nucleotide may be associated with both color channels, and a fourth nucleotide may be associated with neither color channel.

The objective lens 134 also captures fluorescent light emitted by the fluoresced dye molecules in the flow cell 136. Upon capturing this emitted light, the objective lens 134 collects and conveys collimated light that includes the two color channels. This emitted light then propagates back along the path in which the original, excitation illumination arrived from the illumination source 110. It is noted that there is little to no interference expected between the emitted and excitation illumination along this path because of the lack of coherence between the emitted light and excitation illumination. That is, the emitted light is a result of a separate source, namely that of the fluorescent dye in contact with the sample material in the flowcell 136.

The emitted light, upon reflection by the mirror 132, is incident on the excitation/emission dichroic filter 130. The filter 130 transmits the emitted light to a blue/green dichroic filter 138.

In some implementations, a blue/green dichroic filter 138 transmits illumination associated with the blue color channel and reflects illumination associated with the green color channel. In some implementations, the blue/green dichroic filter 138 is selected such that the dichroic filter 138 reflects emitted illumination to an optical subsystem 156 that is within the defined green wavelength band and transmits emitted illumination to an optical subsystem 158 that is within the defined blue wavelength band, as discussed above. The optical subsystem 156 includes a tube lens 142, a filter 144, and the image detector 146. The optical subsystem 158 includes a tube lens 148, a filter 150, and the image detector 154.

In some implementations, the dichroic filter 138 and the dichroic filter 120 operate similarly to each other (e.g., both may reflect light of one color and transmit light of another color). In other implementations, the blue/green dichroic filter 138 and the dichroic filter 120 operate differently from each other (e.g., the dichroic filter 138 may transmit light of a color that the dichroic filter 120 reflects, and vice versa).

Assuming that the blue/green dichroic filter 138 transmits emitted illumination included in the blue color channel, the emitted illumination included in the green color channel may be reflected from the blue/green dichroic filter 138 into the optical subsystem 156. The mirror 140 then reflects the emitted illumination included in the green color channel to incidence on the tube lens 142 of the optical subsystem 156. The filter 144 of the optical subsystem 156 is then a green filter designed to transmit wavelengths in the green color channel of the emitted illumination and absorb or reflect all other wavelengths. The filter 144 may provide additional filtering not available at the blue/green dichroic filter 138. For example, if the blue/green dichroic filter 138 reflects a relatively broad wavelength range of green light, the filter 144 may further restrict that wavelength range so that only a relatively narrower wavelength range of green light reaches the image detector 146. The filter 144 may block any leaked excitation light and/or define a relatively tight wavelength band.

Simultaneously, the blue/green dichroic filter 138 transmits emitted illumination included in the blue color channel to the incidence on the tube lens 148 of the optical subsystem 158. The filter 150 of the optical subsystem 158 is a blue filter designed to transmit wavelengths in the blue color channel of the emitted illumination and absorb or reflect all other wavelengths. The filter 150 may provide additional filtering not available at the blue/green dichroic filter 138. For example, if the blue/green dichroic filter 138 transmits a relatively broad wavelength range of blue light, the filter 150 may further restrict that wavelength range so that only a relatively narrower wavelength range of blue light reaches the image detector 154. The filter 150 may block any leaked excitation light and/or define a relatively tight wavelength band.

In some implementations, and as shown in FIG. 2, the emitted illumination included in the blue color channel encounters a mirror 152 prior to the image detector 154. In example shown, the optical path in the optical subsystem 158 is angled so that the illumination system 100 as a whole may satisfy space or volume requirements. In some implementations, both such subsystems 156 and 158 have optical paths that are angled. In some implementations, neither of the optical paths in the subsystem 156 nor 158 is angled. As such, one or more of multiple optical subsystems can have at least one angled optical path.

Each tube lens 142 and 148 focuses the emitted illumination incident upon it onto respective image detectors 146 and 154. Each detector 146 and 154 includes, in some implementations, a charged coupled device (CCD) array. In some implementations, each image detector 146 and 154 includes a complementary metal-oxide semiconductor (CMOS) sensor.

As stated previously, the illumination system 100 is not required to be as shown in FIG. 2. For example, each of the mirrors 128, 132, 140 may be replaced with a prism or some other optical device that changes the direction of illumination. Each lens may be replaced with a diffraction grating, a diffractive optic, a Fresnel lens, or some other optical device that produces collimated or focused illumination from incident illumination. Furthermore, the illumination system 100 may be designed for separation over different wavelength bands other than blue/green, e.g., red/green or blue/red. Several blue, green, and red dyes discussed herein are further detailed in Section III entitled “Example Fluorescent Dyes” below.

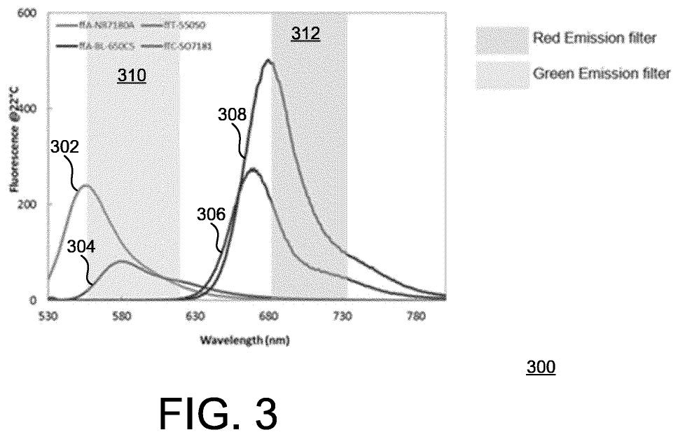

FIG. 3 is a diagram 300 including plots of emission spectra of red and green dyes according to an example implementation. Fluorescence is measured against the vertical axis and wavelength is indicated on the horizontal axis. Fluorescence can be measured in terms of the intensity of emitted light. In some implementations, one or more ways of determining light intensity can be used. For example, an arbitrary intensity unit relative to a calibrated benchmark can be used. Spectra 302 and 304 can be characterized as green dyes, and spectra 306 and 308 can be characterized as red dyes. The diagram 300 includes color channels 310 and 312. The color channel 310 can be associated with a green emission filter. For example, the color channel 310 can be considered a green color channel. The color channel 312 can be associated with a red emission filter. For example, the color channel 312 can be considered a red color channel.

Spectral crosstalk between channels can be a problem. Crosstalk can occur both when the color channels are illuminated sequentially and simultaneously. In some implementations, crosstalk of the lower wavelength channel into the higher wavelength channel can be considered a worse scenario. For example, this can involve the spectrum 302 or 304 spilling into the color channel 312. Here, the spectra 302-308 may have 2.4% crosstalk in a sequential illumination, and 2.8% crosstalk in a simultaneous illumination. For example, this can be considered relatively minimal crosstalk difference between simultaneous and sequential acquisition.

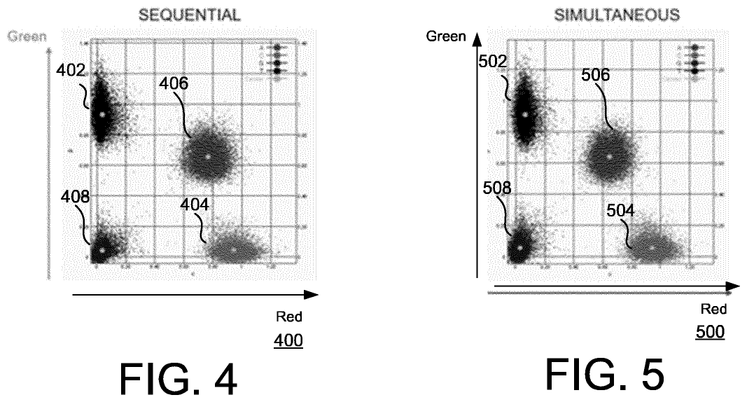

FIG. 4 is a scatterplot 400 illustrating a two-channel sequencing analysis having sequential imaging using green and red dyes of FIG. 3. The amount of emitted light detected in the green channel is indicated on the vertical axis and the amount of emitted light detected in the red channel is indicated on the horizontal axis. An emission 402 corresponds to a substantive emission in the green channel and little or no emission in the red channel. An emission 404 corresponds to a substantive emission in the red channel and little or no emission in the green channel. An emission 406 corresponds to substantive emissions in both the green and red channels. An emission 408 corresponds to little or no emission in both the green and red channels. As such, the emission 908 is an example of a fluorescent dye that does not emit substantial light within the wavelength band of the green channel, and that does not emit substantial light within the wavelength band of the red channel.

Each of the emissions 402-408 can correspond to detection of a corresponding nucleotide. For example, the emission 402 can correspond to detection of thymine. For example, the emission 404 can correspond to detection of cytosine. For example, the emission 406 can correspond to detection of adenine. For example, the emission 408 can correspond to detection of guanine. In the sequential imaging of the present example, the emissions 402-408 are relatively separate from each other and show minimal or negligible crosstalk.

FIG. 5 is a scatter plot illustrating a two-channel sequencing analysis having simultaneous multiplexed imaging using green and red dyes of FIG. 3. The amount of emitted light detected in the green channel is indicated on the vertical axis and the amount of emitted light detected in the red channel is indicated on the horizontal axis. An emission 502 corresponds to a substantive emission in the green channel and little or no emission in the red channel. An emission 504 corresponds to a substantive emission in the red channel and little or no emission in the green channel. An emission 506 corresponds to substantive emissions in both the green and red channels. An emission 508 corresponds to little or no emission in both the green and red channels. Each of the emissions 502-508 can correspond to detection of a corresponding nucleotide. For example, the emission 502 can correspond to detection of thymine. For example, the emission 504 can correspond to detection of cytosine. For example, the emission 506 can correspond to detection of adenine. For example, the emission 508 can correspond to detection of guanine. In the simultaneous imaging of the present example, the emissions 502-508 are relatively separate from each other and show minimal or negligible crosstalk.

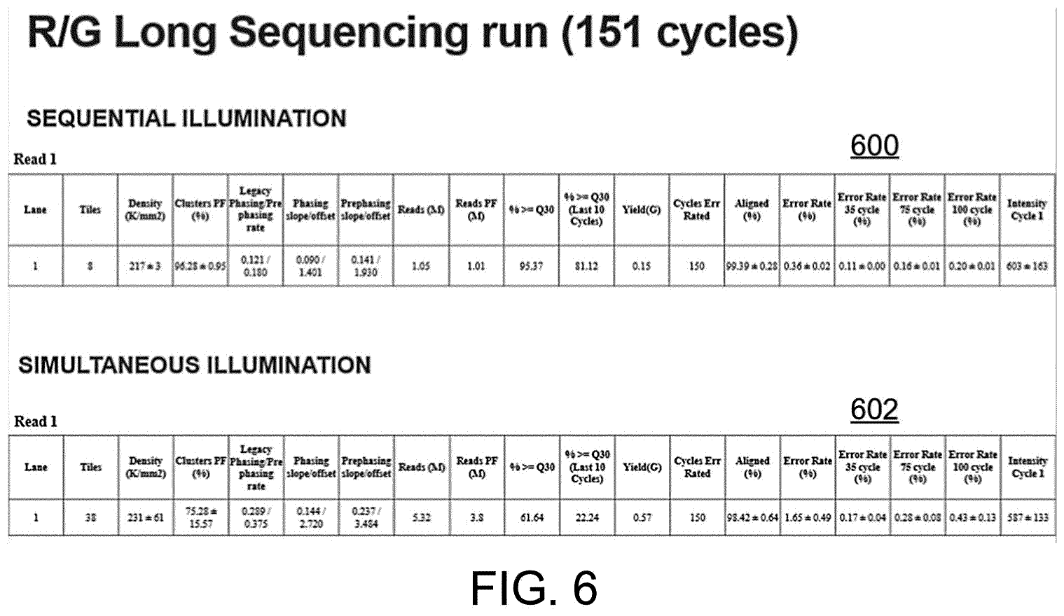

FIG. 6 is a diagram depicting metrics for the two-channel sequencing analyses of FIGS. 4-5. A metric 600 relates to the sequential illumination and a metric 602 relates to the simultaneous illumination.

That is, examples described above indicate that the level of crosstalk in a red/green system may be relatively low, even in a simultaneous acquisition. With other color channels, however, the amount of crosstalk may be more challenging.

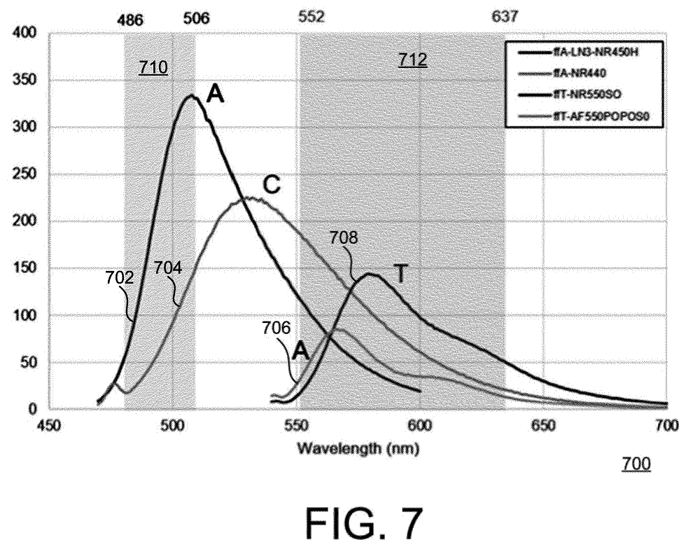

FIG. 7 is a diagram 700 including plots of emission spectra of blue and green dyes according to an example implementation. Fluorescence is measured against the vertical axis and wavelength is indicated on the horizontal axis. Spectra 702 and 704 can be characterized as blue dyes, and spectra 706 and 708 can be characterized as green dyes. For example, the spectrum 702 can correspond to detection of adenine in blue illumination. For example, the spectrum 704 can correspond to detection of cytosine. For example, the spectrum 706 can correspond to detection of adenine in green illumination. For example, the spectrum 708 can correspond to detection of thymine.

The diagram 700 includes color channels 710 and 712. The color channel 710 can be associated with a blue emission filter. For example, the color channel 710 can be considered a blue color channel. The color channel 712 can be associated with a green emission filter. For example, the color channel 712 can be considered a green color channel.

The diagram 700 shows that the spectrum 704, which may correspond to the blue emission for identifying a cytosine base, spills over significantly in the color channel 712. In some implementations, this may occur because the separation between the green and blue excitation wavelengths (which may be, e.g., about 70 nm) is relatively much smaller than the separation between the red and green wavelengths (which may be, e.g., about 140 nm, see FIG. 3). That is, the emission spectra of the blue dye emits wavelength components that overlap with the emission spectra of the green dye. The fluorescent emissions in the blue/green scenario (e.g., diagram 700) may therefore be much closer than in the red/green scenario (e.g., diagram 300). In sequential illumination with blue/green illumination, the amount of crosstalk may be relatively minimal or negligible. In simultaneous illumination, however, the crosstalk may be relatively significant. For example, the crosstalk may be about 40%.

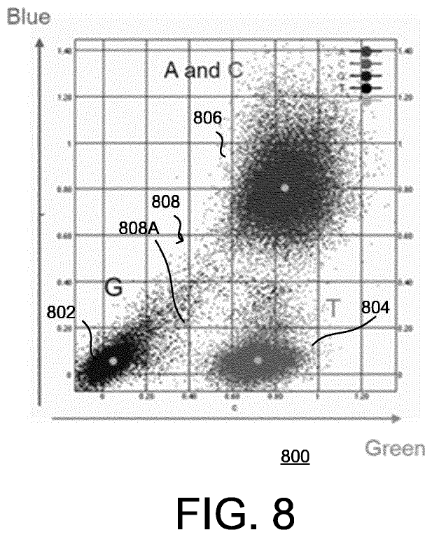

FIG. 8 is a scatterplot 800 illustrating a two-channel sequencing analysis having simultaneous multiplexed imaging using blue and green dyes of FIG. 7. The amount of emitted light detected in the blue channel is indicated on the vertical axis and the amount of emitted light detected in the green channel is indicated on the horizontal axis. An emission 802 corresponds to little or no emission in both the blue and green channels. As such, the emission 802 is an example of a fluorescent dye that does not emit substantial light within the wavelength band of the blue channel, and that does not emit substantial light within the wavelength band of the green channel. An emission 804 corresponds to a substantive emission in the green channel and little or no emission in the blue channel. An emission 806 corresponds to substantive emissions in both the blue and green channels. An emission 808 is spread out in the scatterplot 800 and coincides with part of at least the emissions 802 and 806. A centroid 808A of the emission 808 is indicated. Each of the emissions 802-808 can correspond to detection of a corresponding nucleotide. For example, the emission 802 can correspond to detection of guanine. For example, the emission 804 can correspond to detection of thymine. For example, the emission 806 can correspond to detection of adenine. For example, the emission 808 can correspond to detection of cytosine. In the simultaneous imaging of the present example, the emissions 802-808 have relatively significant crosstalk.

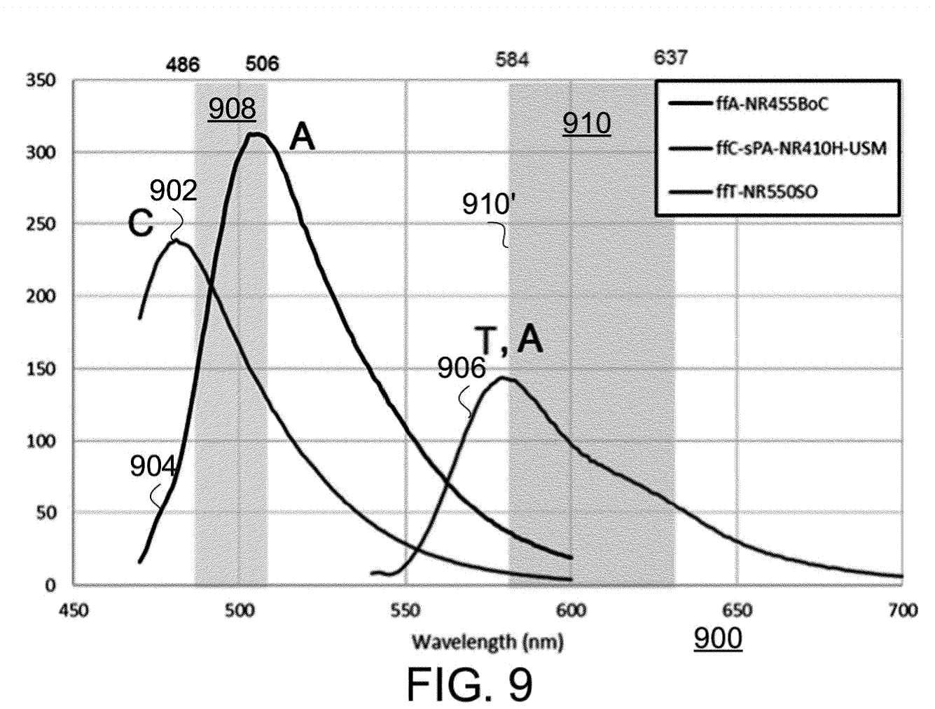

FIG. 9 is another diagram 900 including plots of emission spectra of alternative blue and green dyes according to an example implementation. Fluorescence is measured against the vertical axis and wavelength is indicated on the horizontal axis. Spectra 902 and 904 can be characterized as blue dyes, and spectrum 906 can be characterized as a green dye. The diagram 900 includes color channels 908 and 910. The color channel 908 can be associated with a blue emission filter. For example, the color channel 908 can be considered a blue color channel. The color channel 910 can be associated with a green emission filter. For example, the color channel 910 can be considered a green color channel. Each of the spectra 902-906 can correspond to detection of a corresponding nucleotide. For example, the spectrum 902 can correspond to detection of cytosine. For example, the spectrum 904 can correspond to detection of adenine. For example, the spectrum 906 can correspond to detection of thymine or adenine.

The spectral emissions in the diagram 900 show a dye that supports simultaneous multi-color imaging. For example, in contrast with diagram 700 in FIG. 7, the peak of the spectrum 902, which corresponds to the blue emission of the cytosine base sequencing dye, is heavily blue-shifted. Here, the spectrum 904 has a peak in the color channel 908, whereas a peak of the spectrum 902 is not within the spectrum 908. A peak of the spectrum 906 is located slightly below the lower end of the color channel 910. The diagram 900 may indicate a relatively minimal or negligible crosstalk in a sequential illumination. For example, the diagram 900 may indicate about 12% crosstalk in a simultaneous illumination.

The relatively low level of crosstalk in the diagram 900 may correlate with the respective dyes being sufficiently separate from each other. In some implementations, separation can be defined based on peak or mean wavelength of emission spectra. A peak wavelength can correspond to a local or global maximum of intensity of the emitted light. A mean wavelength can correspond to an average wavelength within the range of the emission spectrum. In some implementations, dyes can be selected so that their respective peak or mean wavelengths have at least a predefined separation from each other. For example, the peak wavelength of the spectrum 902 can have at least a predefined separation from the peak wavelength of the spectrum 906. As another example, the peak wavelength of the spectrum 904 can have at least a predefined separation from the peak wavelength of the spectrum 906.

In some implementations, separation can be defined based on amount of light in overlapping wavelength ranges. A left edge 910′ of the color channel 910 can correspond to a particular wavelength of the wavelength range of the color channel 910. It may be desirable to ensure that the spectra 902 or 904 do not extend significantly into the color channel 910. In some implementations, a separation between the respective fluorescent dyes can be defined so that the spectrum 902 or 904 includes at most a predefined amount of light at or above the wavelength corresponding to the edge 910′. The predefined amount can be defined as an absolute number (e.g., as a upper threshold on the amount of emitted light, or its intensity) or as a relative number (e.g., as a proportion of the total amount of fluorescent light emitted by the dye.

The blue dyes, and variants thereof, described in reference to FIGS. 9-16 are described in greater detail below.

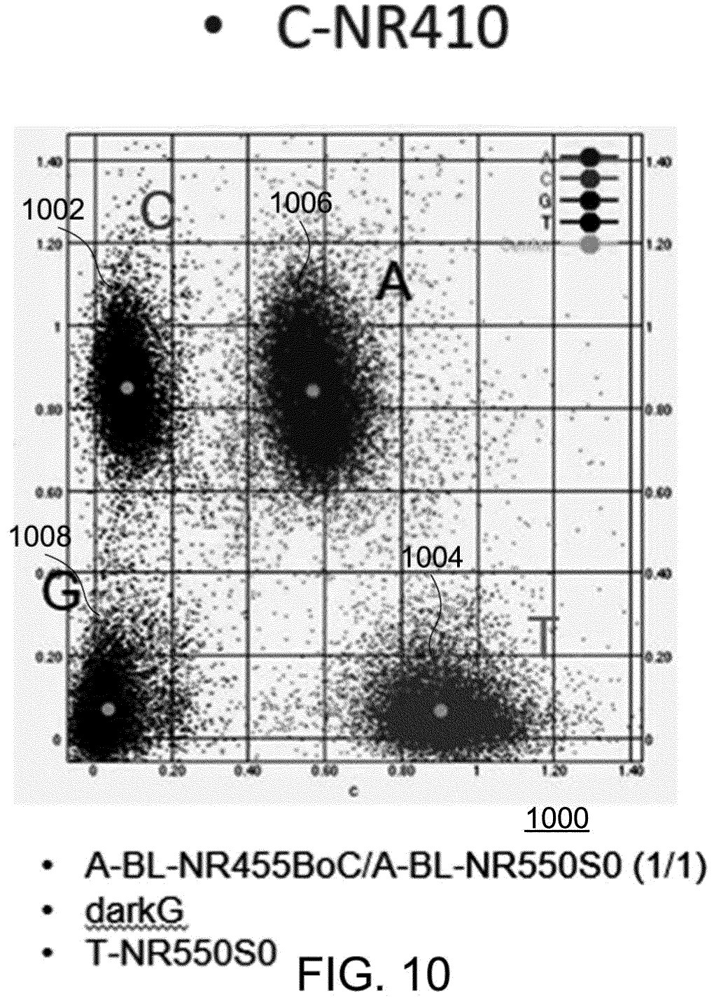

FIG. 10 is a scatterplot 1000 illustrating a two-channel sequencing analysis having simultaneous multiplexed imaging using blue and green dyes of FIG. 9. The amount of emitted light detected in the blue channel is indicated on the vertical axis and the amount of emitted light detected in the green channel is indicated on the horizontal axis. An emission 1002 corresponds to a substantive emission in the blue channel and little or no emission in the green channel. An emission 1004 corresponds to a substantive emission in the green channel and little or no emission in the blue channel. An emission 1006 corresponds to substantive emissions in both the blue and green channels. An emission 1008 corresponds to little or no emission in both the blue and green channels. Each of the emissions 1002-1008 can correspond to detection of a corresponding nucleotide. For example, the emission 1002 can correspond to detection of cytosine. For example, the emission 1004 can correspond to detection of thymine. For example, the emission 1006 can correspond to detection of adenine. For example, the emission 1008 can correspond to detection of guanine. In the simultaneous imaging of the present example, the emissions 1002-1008 are relatively separate from each other and show minimal or negligible crosstalk

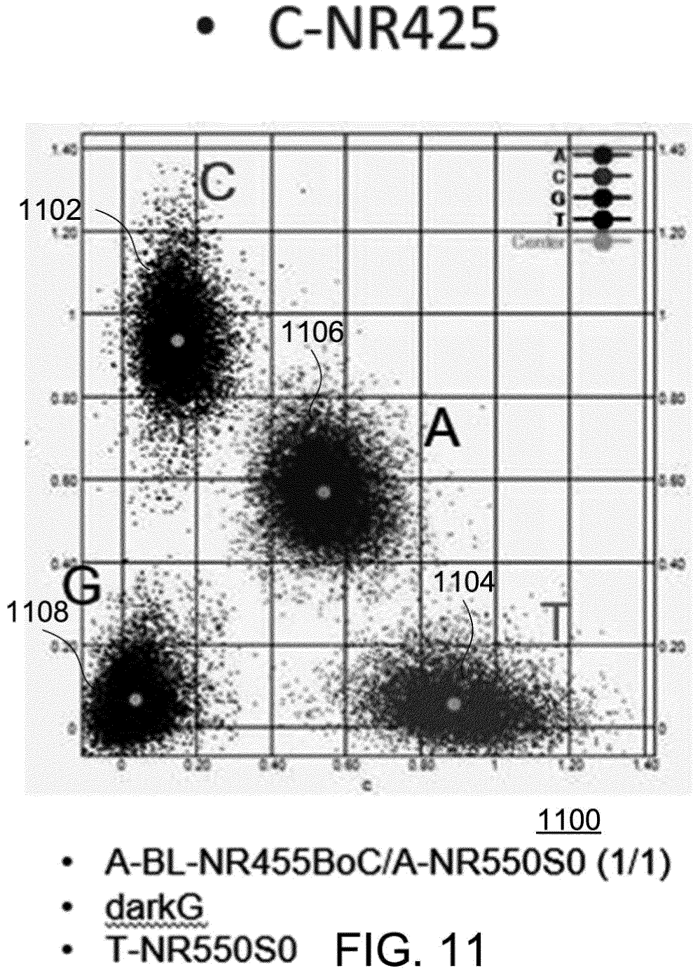

FIG. 11 is a scatterplot 1100 illustrating a two-channel sequencing analysis having simultaneous multiplexed imaging using other blue and green dyes. The amount of emitted light detected in the blue channel is indicated on the vertical axis and the amount of emitted light detected in the green channel is indicated on the horizontal axis. An emission 1102 corresponds to a substantive emission in the blue channel and little or no emission in the green channel. An emission 1104 corresponds to a substantive emission in the green channel and little or no emission in the blue channel. An emission 1106 corresponds to substantive emissions in both the blue and green channels. An emission 1108 corresponds to little or no emission in both the blue and green channels. Each of the emissions 1102-1108 can correspond to detection of a corresponding nucleotide. For example, the emission 1102 can correspond to detection of cytosine. For example, the emission 1104 can correspond to detection of thymine. For example, the emission 1106 can correspond to detection of adenine. For example, the emission 1108 can correspond to detection of guanine. In the simultaneous imaging of the present example, the emissions 1102-1108 are relatively separate from each other and show minimal or negligible crosstalk.

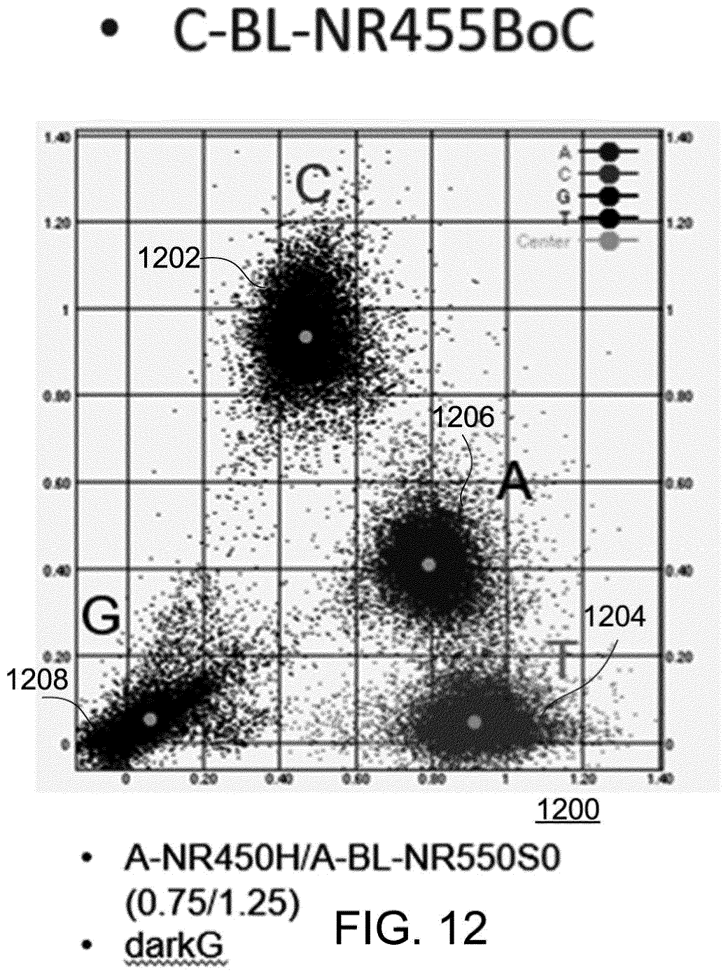

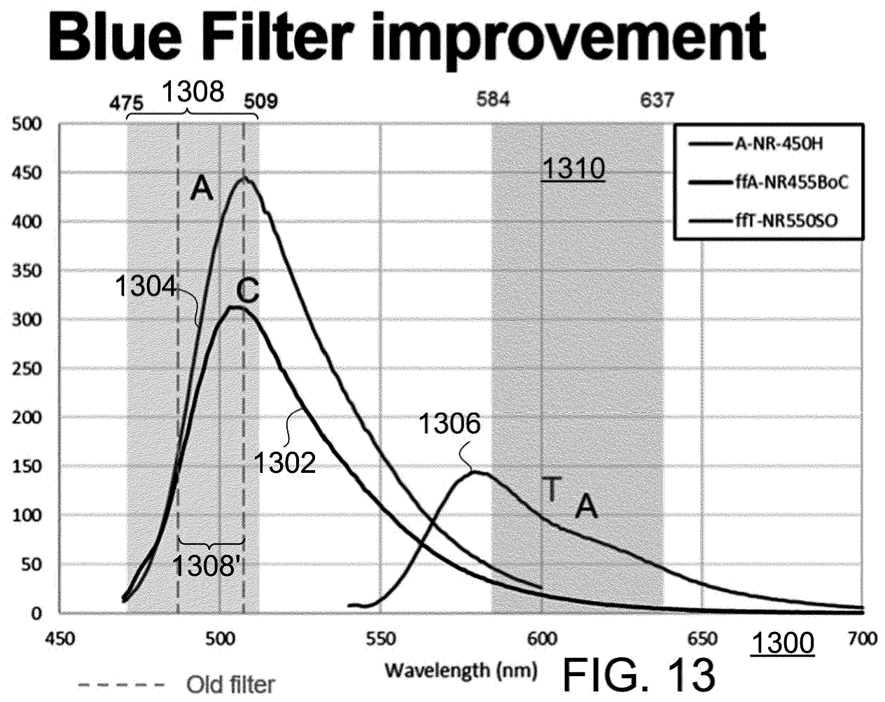

FIG. 12 is a scatterplot 1200 illustrating a two-channel sequencing analysis having simultaneous multiplexed imaging using still other blue and green dyes. The amount of emitted light detected in the blue channel is indicated on the vertical axis and the amount of emitted light detected in the green channel is indicated on the horizontal axis. An emission 1202 corresponds to a substantive emission in the blue channel and little or no emission in the green channel. An emission 1204 corresponds to a substantive emission in the green channel and little or no emission in the blue channel. An emission 1206 corresponds to substantive emissions in both the blue and green channels. An emission 1208 corresponds to little or no emission in both the blue and green channels. Each of the emissions 1202-1208 can correspond to detection of a corresponding nucleotide. For example, the emission 1202 can correspond to detection of cytosine. For example, the emission 1204 can correspond to detection of thymine. For example, the emission 1206 can correspond to detection of adenine. For example, the emission 1208 can correspond to detection of guanine. In the simultaneous imaging of the present example, the emissions 1202-1208 are relatively separate from each other and show minimal or negligible crosstalk.

Different color filters can be used. A filter design for simultaneous acquisition can be selected. In some implementations, a green filter emission passband of about 583-660 nm can be used. For example, this can represent a shift compared to another green passband such as 550-637 nm.

FIG. 13 is another diagram 1300 including plots of emission spectra of alternative blue and green dyes and corresponding filter ranges according to an example implementation. Fluorescence is measured against the vertical axis and wavelength is indicated on the horizontal axis. Spectra 1302 and 1304 can be characterized as blue dyes, and spectrum 1306 can be characterized as a green dye. The diagram 1300 includes color channels 1308 and 1310. The color channel 1308 can be associated with a blue emission filter and can contrast with a previous filter 1308′. For example, the color channel 1308 can be considered a blue color channel. The color channel 1310 can be associated with a green emission filter. For example, the color channel 1310 can be considered a green color channel. Each of the spectra 1302-1306 can correspond to detection of a corresponding nucleotide. For example, the spectrum 1302 can correspond to detection of cytosine. For example, the spectrum 1304 can correspond to detection of adenine. For example, the spectrum 1306 can correspond to detection of thymine or adenine.

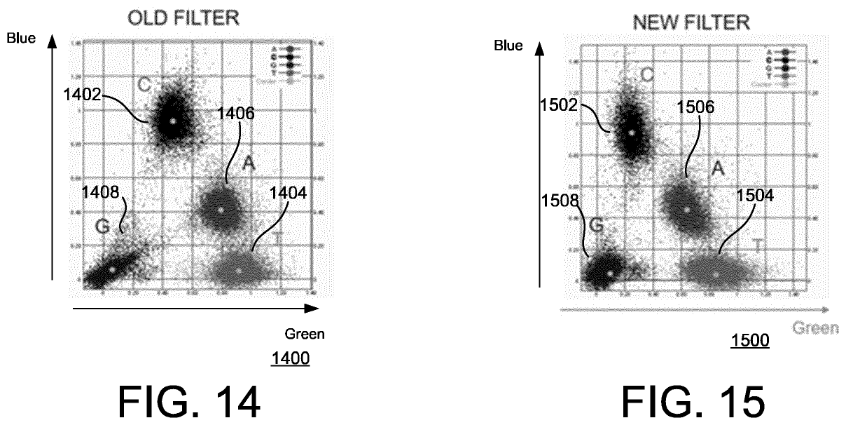

FIG. 14 is a scatterplot 1400 illustrating a two-channel sequencing analysis having simultaneous multiplexed imaging using blue and green dyes of FIG. 13 using a first filter range. For example, the first filter range can correspond to the previous filter 1308′ in FIG. 13. The amount of emitted light detected in the blue channel is indicated on the vertical axis and the amount of emitted light detected in the green channel is indicated on the horizontal axis. An emission 1402 corresponds to a substantive emission in both the blue and green channels. An emission 1404 corresponds to a substantive emission in the green channel and little or no emission in the blue channel. An emission 1406 corresponds to substantive emissions in both the blue and green channels. An emission 1408 corresponds to little or no emission in both the blue and green channels. Each of the emissions 1402-1408 can correspond to detection of a corresponding nucleotide. For example, the emission 1402 can correspond to detection of cytosine. For example, the emission 1404 can correspond to detection of thymine. For example, the emission 1406 can correspond to detection of adenine. For example, the emission 1408 can correspond to detection of guanine. In the simultaneous imaging of the present example, the emissions 1402-1408 are relatively separate from each other and show minimal or negligible crosstalk.

FIG. 15 is a scatterplot 1500 illustrating a two-channel sequencing analysis having simultaneous multiplexed imaging using blue and green dyes of FIG. 13 using a second filter range. For example, the second filter range can correspond to the color channel 1308′ in FIG. 13. The amount of emitted light detected in the blue channel is indicated on the vertical axis and the amount of emitted light detected in the green channel is indicated on the horizontal axis. An emission 1502 corresponds to a substantive emission in both the blue and green channels. An emission 1504 corresponds to a substantive emission in the green channel and little or no emission in the blue channel. An emission 1506 corresponds to substantive emissions in both the blue and green channels. An emission 1508 corresponds to little or no emission in both the blue and green channels. Each of the emissions 1502-1508 can correspond to detection of a corresponding nucleotide. For example, the emission 1502 can correspond to detection of cytosine. For example, the emission 1504 can correspond to detection of thymine. For example, the emission 1506 can correspond to detection of adenine. For example, the emission 1508 can correspond to detection of guanine. In the simultaneous imaging of the present example, the emissions 1502-1508 are relatively separate from each other and show minimal or negligible crosstalk.

Separation can be defined in one or more ways. In some implementations, a wavelength emission separation can be defined based on an amount of emitted light from the spectrum 1304 below a wavelength associated with the color channel 1310. The wavelength emission separation can be defined between the fluorescent dyes so that an emission spectrum of one of the fluorescent dyes includes at most a predefined amount of light at or above a wavelength (e.g., a closest boundary wavelength, or a characteristic wavelength) associated with the other fluorescent dye. For example, the amount can indicate that an amount X (e.g., a percentage of total fluorescence) of the spectrum 1304 occurs below a lower wavelength of the color channel 1310 (e.g., the lower limit of that color channel). In some implementations, the number X in the preceding example can be any suitable number, such as a range of values. For example, the range can be about 0-10% of the fluorescent light. As another example, the range can be about 0.5-5% of the fluorescent light. As another example, the range can be about 0.1-1% of the fluorescent light. In some implementations, the separation can be defined based on a mean or peak wavelength separation between the spectrum 1306 and either of the spectra 1302 or 1304. For example, the spectra 1304 and 1306 can be deemed separate if the mean wavelength of the spectrum 1304 (e.g., the average wavelength of the fluorescent emissions) or the peak wavelength of the spectrum 1304 (e.g., the wavelength at which the intensity of fluorescent light is greatest) is separated from the mean or peak wavelength of the spectrum 1306 by more than a predefined amount. The predefined amount can be an absolute value. For example, the mean or peak wavelengths can be separated by at least about 50-100 nm, such as by about 70 nm. The predefined amount can be a relative value. For example, the mean or peak wavelengths can be separated by at least about 5-20 percent of either mean or peak wavelength, such as by about 13 percent of the lower or higher mean or peak wavelength.

In conclusion, using improvements described herein a multi-color image acquisition can be achieved, which was previously thought extremely challenging and with a very small chance of success. Some more examples of improvements will now be described.

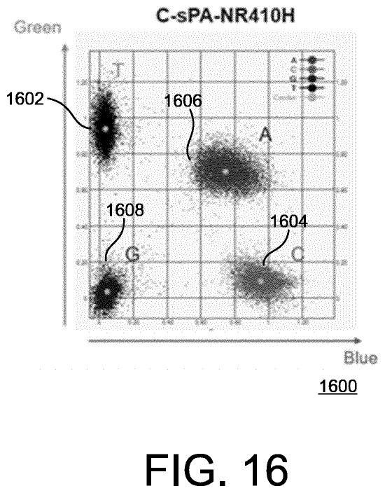

FIG. 16 is a scatterplot 1600 illustrating a two-channel sequencing analysis having simultaneous multiplexed imaging using the blue and green dyes of FIG. 9 and the second filter rage of FIG. 13. The amount of emitted light detected in the green channel is indicated on the vertical axis and the amount of emitted light detected in the blue channel is indicated on the horizontal axis. An emission 1602 corresponds to a substantive emission in the green channel and little or no emission in the blue channel. An emission 1604 corresponds to a substantive emission in the blue channel and little or no emission in the green channel. An emission 1606 corresponds to substantive emissions in both the green and blue channels. An emission 1608 corresponds to little or no emission in both the green and blue channels. Each of the emissions 1602-1608 can correspond to detection of a corresponding nucleotide. For example, the emission 1602 can correspond to detection of thymine. For example, the emission 1604 can correspond to detection of cytosine. For example, the emission 1606 can correspond to detection of adenine. For example, the emission 1608 can correspond to detection of guanine. In the simultaneous imaging of the present example, the emissions 1602-1608 are relatively separate from each other and show minimal or negligible crosstalk.

Each of the above emissions 1602-1608 represents a distribution of intensities collected at one of the two detectors 146 and 154 (FIG. 2) over time. As indicated in the plots of the emission spectra in FIG. 13, the “C” nucleobase is associated with the blue dye, and hence emission 1604 has a large amount of high blue illumination level and low green illumination level. This is how the “C” nucleobase is identified. The “T” nucleobase is identified via emission 1602 having a large amount of green illumination level and low blue illumination level; this is how the “T” nucleobase is identified.

The “A” nucleobase, identified by the emission 1606, has a mixture of high blue and green illumination levels. It is noted that the spectra 1304 and 1306 (FIG. 13) both corresponded to the “A” nucleobase. Similarly, the “G” nucleobase is identified by the emission 1608 having low levels of blue and green illumination.

The emissions 1602-1608, while having distributions about respective mean values with a significant amount of spread, largely do not exhibit significant amounts of crosstalk. In this way, each of the nucleobases may be easily identified.

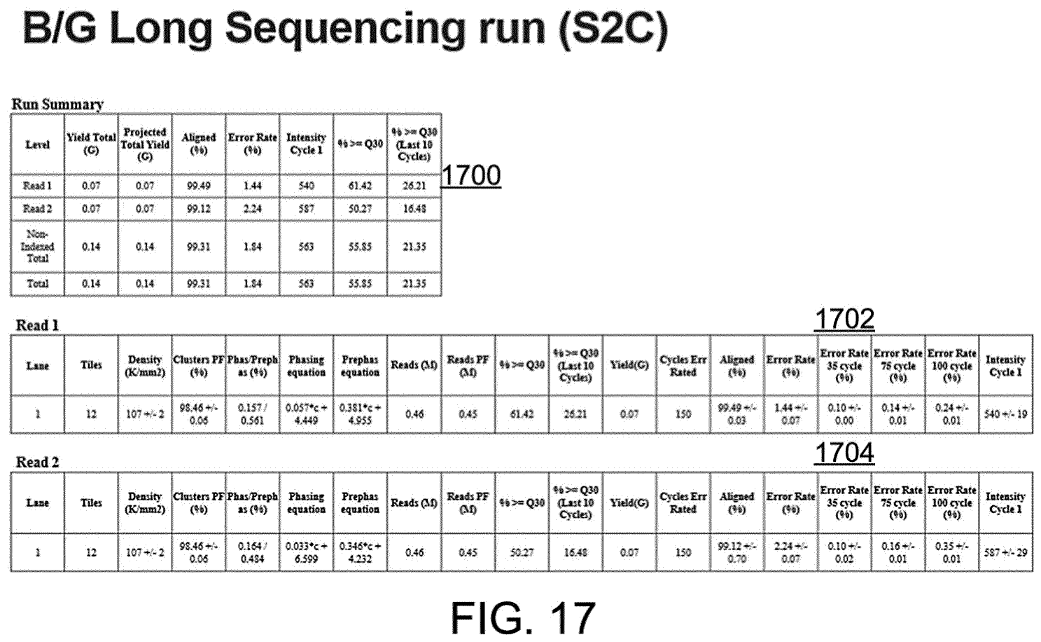

FIG. 17 is a diagram depicting metrics for the two-channel sequencing analysis of FIG. 16. A metric 1700 relates to a run summary. A metric 1702 relates to a first read, and a metric 1704 relates to a second read.

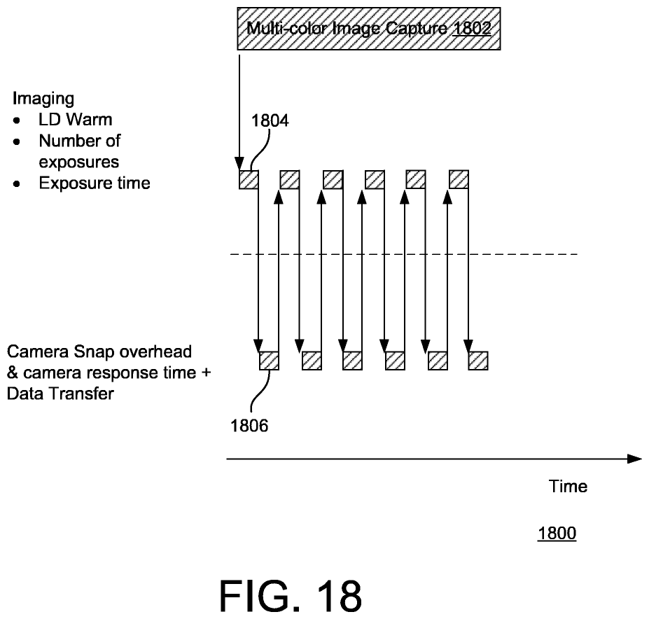

FIG. 18 is a diagram representing a timeline 1800 of example sequential steps that may be involved in producing and analyzing multiplexed fluorescence images as part of the improved techniques described herein. The timeline 1800 can be used with one or more examples described elsewhere herein. Progression of time is measured against the horizontal axis and respective operations are indicated along the vertical axis.

A multi-color image capture 1802 as schematically illustrated can include one or more imaging time blocks 1804, and one or more camera-related time blocks 1806. In some implementations, the imaging time block 1804 can correspond to the time required for the system to perform warming of a laser diode, arrange for one or more exposures, and the exposure time for the exposure(s). After the imaging time block 1804, the camera-related time block 1806 can follow. For example, the camera-related time block 1806 can include the overhead time, the camera response time related to the individual camera snap(s), and the time for data transfer. After the camera-related time block 1806, another one of the imaging time block 1804 can follow. As such, the multi-color image capture 1802 can include a sequence alternating between the imaging time block 1804 and the camera-related time block 1806. For example, this can involve introduction of the dye, exposure time, and camera snap for the image.

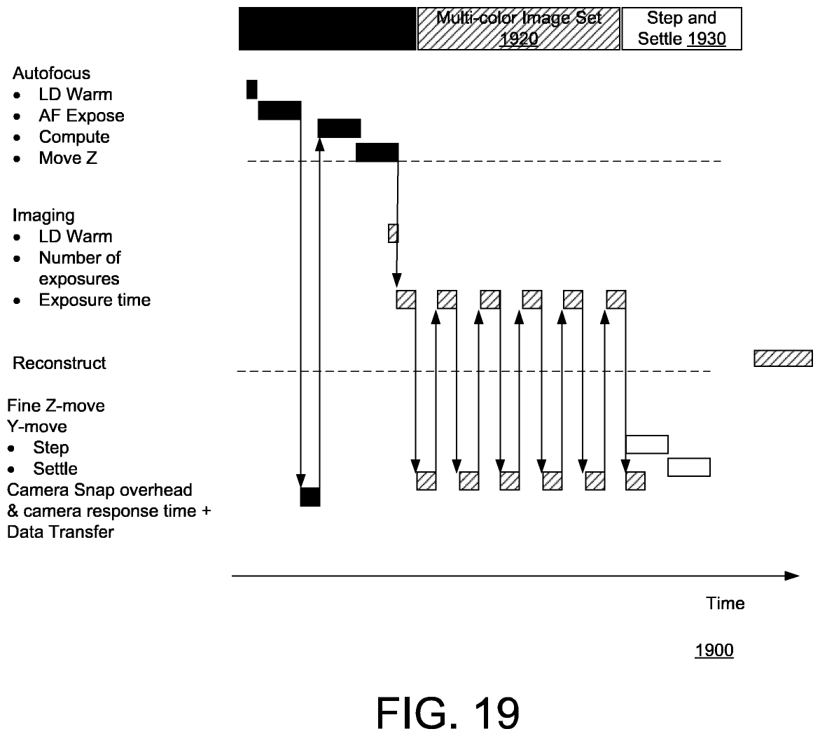

FIG. 19 is a diagram representing a timeline 1900 of example sequential steps that may be involved in producing and analyzing multiplexed fluorescence images as part of the improved techniques described herein. As shown in FIG. 19, the timeline 1900 includes an autofocus process 1910, a multi-color image set acquisition process 1920, and a step and settle process 1930. The horizontal axis represents elapsed time. Some examples below will also refer to FIG. 2 for illustrative purposes only.

The autofocus process 1910 begins the timeline 1900. First, the laser diodes are warmed and an autofocus exposure is generated. Based on the camera (i.e., detector) snap overhead, response time, and data transfer time, a determination is made to move the objective lens 134 along its axis (i.e., the “z” direction) to establish the position of the objective lens 134 at which a focused beam of illumination is incident at a desired object plane relative to the flowcell 136.

After this position of the objective lens 134 has been set, the multi-color image set acquisition process 520 may begin. For example, this can involve capturing the image(s) using blue and green color channels, or red and green color channels, or another selection of color channels. For each of the blue and green image detectors 146 and 154, after the laser diodes have warmed, the sample is then illuminated for a predetermined time to fluoresce the one or more dyes.

A multiplexed fluorescence image is acquired by the image detectors 146 and 154 and the resulting data can be transferred to a processing system. As shown in FIG. 19, this process is here repeated for both detectors six times, for acquiring six images on each detector. In some implementations, the image set acquisition process may be repeated several times, e.g., two, three, four, five, seven, eight, nine, ten, eleven, twelve, and higher, depending upon the implementation. The data transferred can be used in a reconstruction of the DNA sequence. A reconstruction and/or determination of a genetic sequence (e.g., a DNA sequence) can occur after all images are captured and nucleotide bases have been called.

After the multi-color (e.g., blue and green) images and their data have been acquired, a different portion of the flowcell 136 is moved into position for imaging. Here, when the flowcell 136 is on a stage, the stage is moved over by a tile, which can be a defined subdivision for the flowcell 136, and then a step and settle process 1930 occurs to allow the flowcell 136 and any other mechanical components to become substantially stationary before the next imaging process occurs. That is, the flowcell 136 is advanced (stepped) on a stage, and after moving the flowcell 136, some time is allowed for the liquid in the flowcell 136 to settle.

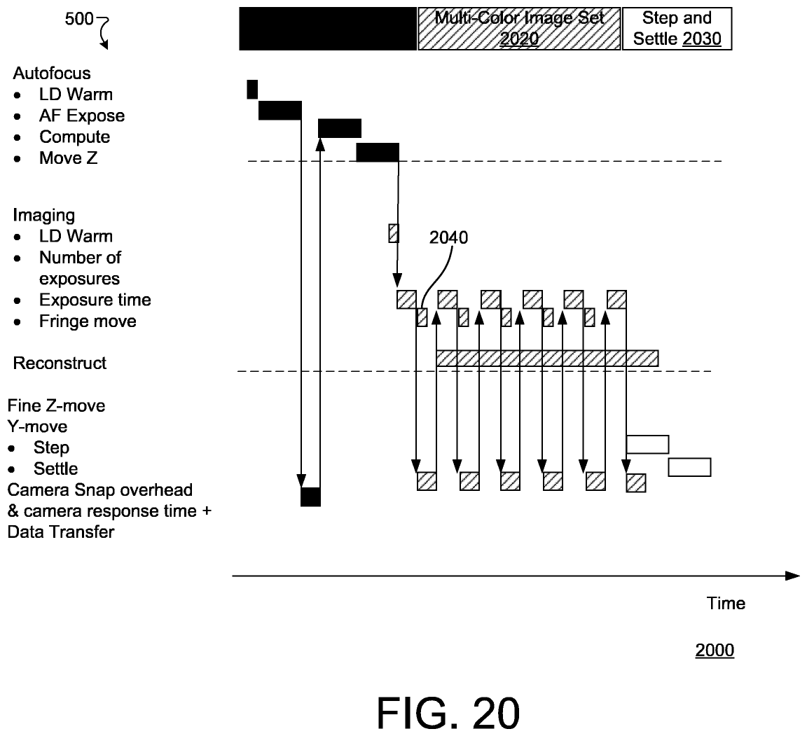

FIG. 20 is a diagram representing a timeline 2000 of example sequential steps that may be involved in producing and analyzing multiplexed fluorescence images as part of the improved techniques described herein. As shown in FIG. 20, the timeline 2000 includes an autofocus process 2010, a multi-color (e.g., blue and green) image set acquisition process 2020, and a step and settle process 2030. The horizontal axis represents elapsed time. Some examples below will also refer to FIG. 2 for illustrative purposes only.

The autofocus process 2010 begins the timeline 2000. First, the laser diodes are warmed and an autofocus exposure is generated. Based on the camera (i.e., detector) snap overhead, response time, and data transfer time, a determination is made to move the objective lens 134 along its axis (i.e., the “z” direction) to establish the position of the objective lens 134 at which a focused beam of illumination is incident at a desired object plane relative to the flowcell 136.

After this position of the objective lens 134 has been set, the multi-color image set acquisition process 2020 may begin. For each of the blue and green image detectors 146 and 154, after the laser diodes have warmed, the sample is then illuminated for a predetermined time to fluoresce the one or more dyes. In implementations that utilize structured illumination microscopy (SIM), a grating or other SIM component can be moved to modify the phase of one or more fringes at 2040. The one or more fringes may occur according to some periodicity. These fringes are moved in order to provide illumination to a different part of the sample while blocking illumination at others. A multiplexed fluorescence image is acquired by the image detectors 146 and 154 and the resulting data can be transferred to a processing system. As shown in FIG. 20, this process is here repeated for both detectors six times, for acquiring six images on each detector. In some implementations, the image set acquisition process may be repeated several times, e.g., two, three, four, five, seven, eight, nine, ten, eleven, twelve, and higher, depending upon the implementation. During these exposures and captures, the data transferred is used in a reconstruction of the DNA sequence.

After the multi-color images and their data have been acquired, a different portion of the flowcell 136 is moved into position for imaging. Here, when the flowcell 136 is on a stage, the stage is moved over by a tile, which can be a defined subdivision for the flowcell 136, and then a step and settle process 2030 occurs to allow the flowcell 136 and any other mechanical components to become substantially stationary before the next imaging process occurs. That is, the flowcell 136 is advanced (stepped) on a stage, and after moving the flowcell 136, some time is allowed for the liquid in the flowcell 136 to settle.

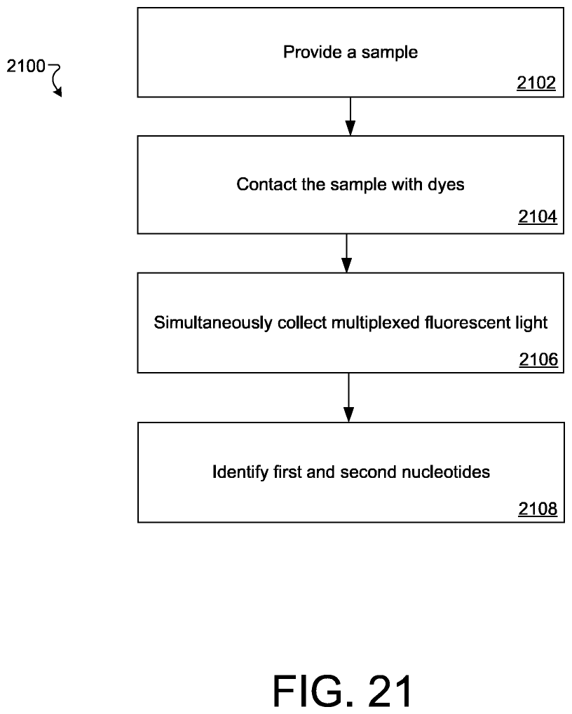

FIG. 21 is a flow chart illustrating a method 2100 of performing a sequencing operation according to the techniques described herein. The method 2100 can be performed using the illumination system 100 described herein. The method 2100 can include more or fewer operations than shown. Two or more of the operations of the method 2100 can be performed in a different order unless otherwise indicated. Some aspects of other examples described herein will be referenced for illustrative purposes.

At 2102, a sample including a first nucleotide and a second nucleotide is provided. For example, such nucleotides may be part of a sample material in the flowcell 136 in FIG. 2.

At 2104, the sample is contacted with a first fluorescent dye and a second fluorescent dye. The first fluorescent dye emits first emitted light within a first wavelength band responsive to a first excitation illumination light, and the second fluorescent dye emits second emitted light within a second wavelength band responsive to a second excitation illumination light. For example, the first fluorescent dye may include a blue dye having the spectrum 1304 shown in FIG. 13, while the second dye may be the green dye with the spectrum 1306 shown in FIG. 13.

At 2106, multiplexed fluorescent light is simultaneously collected. The multiplexed fluorescent light comprises at least the first emitted light and the second emitted light. The first emitted light can be a first color channel corresponding to the first wavelength band, and the second emitted light can be a second color channel corresponding to the second wavelength band. For example, blue and green color channels can be used. As another example, blue, green and red color channels can be used. The peak of one dye emission (e.g., that of a blue dye) should have sufficient separation over the light spectrum from the peak of another dye emission (e.g., that of a green dye) so that the lower wavelength emitted light (e.g., blue) does not spill over in the other (e.g., green) emission detection channel. This would cause what is sometimes referred to as crosstalk where emitted light (e.g., the tail of a spectrum) is detected by the detector of the other color channel. In cases where a spectrum has a relatively long tail, a starting point of the other emission filter can be moved to eliminate or reduce the amount of crosstalk.

At 2108, the first and second nucleotides can be identified. The first nucleotide can be identified based on the first wavelength band of the first color channel, and the second nucleotide can be identified based on the second wavelength band of the second color channel.

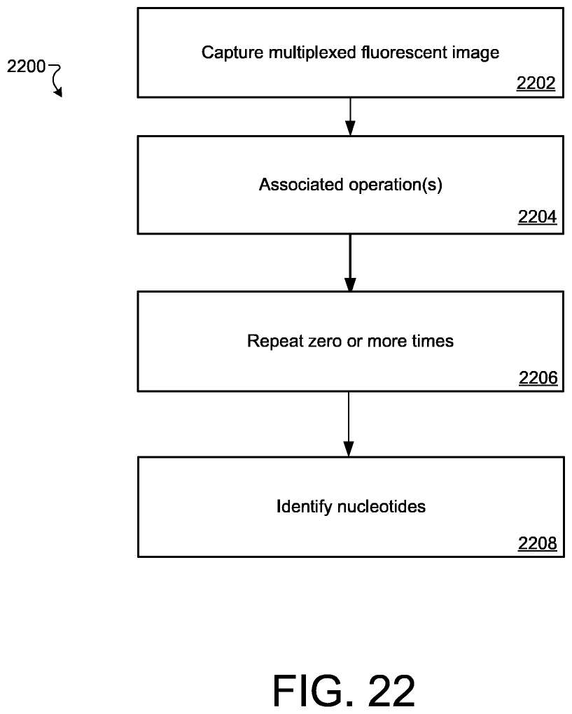

FIG. 22 is a flow chart illustrating a method 2200 of performing a sequencing operation according to the techniques described herein. The method 2200 can be performed using the illumination system 100 described herein. The method 2200 can include more or fewer operations than shown. Two or more of the operations of the method 2200 can be performed in a different order unless otherwise indicated. Some aspects of other examples described herein will be referenced for illustrative purposes.

At 2202, a multiplexed fluorescent image can be captured. In some implementations, this can be done based on simultaneous illumination of a dye-tagged sample with multiple types of illuminating light, and capturing of images from emission light in more than one color channel (including, but not limited to, in blue and green color channels). For example, the imaging time block(s) 1804 in FIG. 18 can correspond to the present operation(s).

At 2204, one or more operations associated with the image capture can be performed. In some implementations, this can include camera response time, data transfer, and/or overhead operations. For example, the camera-related time block 1806 can correspond to the present operation(s).

At 2206, zero or more repetitions of the operations at 2202 and 2204 can be performed. In some implementations, the operations at 2202 and 2204 can be performed alternatingly in multiple cycles. For example, performance six times can be implemented to acquire six images on each detector (see, e.g., FIG. 18).

At 2208, nucleotides can be identified based on the multiplexed fluorescent image(s). For example, each nucleotide can be identified based on a corresponding color channel.

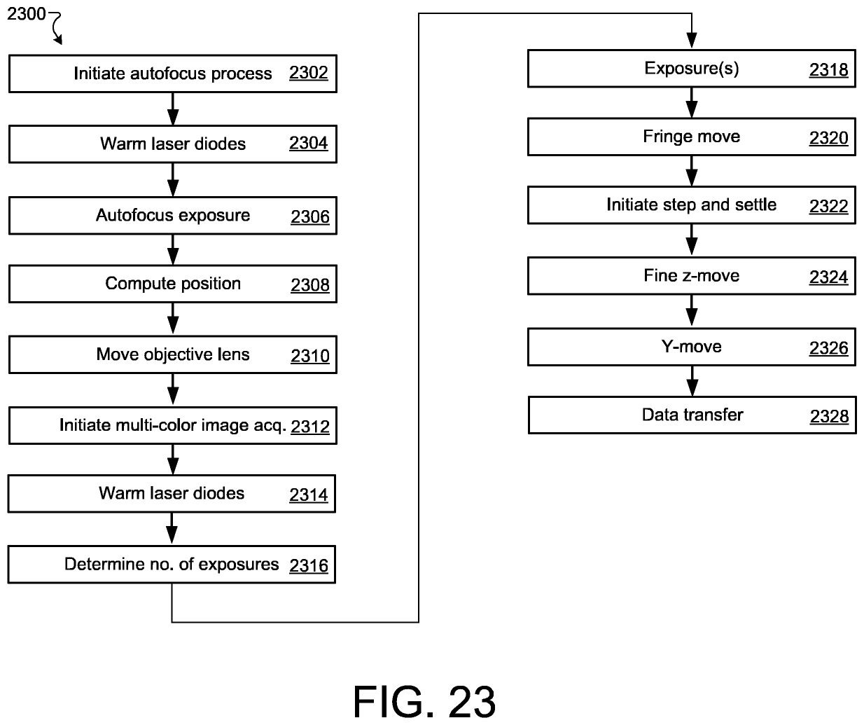

FIG. 23 is a flow chart illustrating a method 2300 of performing a sequencing operation according to the techniques described herein. The method 2300 can be performed using the illumination system 100 described herein. The method 2300 can include more or fewer operations than shown. Two or more of the operations of the method 2300 can be performed in a different order unless otherwise indicated. Some aspects of other examples described herein will be referenced for illustrative purposes.

At 2302, and autofocus process can be initiated. In some implementations, the autofocus process 2010 (FIG. 20) is initiated.

At 2304, one or more laser diodes can be warmed. In some implementations, this is part of the autofocus process.

At 2306, an autofocus exposure can be performed. In some implementations, this is part of the autofocus process.

At 2308, a position can be computed. In some implementations, this can include a determination of whether to move the objective lens. For example, it can be determined whether, and by how much, to move the objective lens along the z-direction. This can be part of the autofocus process.

At 2310, the objective lens can be moved. In some implementations, this can be part of the autofocus process.

At 2312. a multi-color image acquisition can be initiated. In some implementations, this can involve the acquisition of more than one multiplexed fluorescent image.

At 2314, one or more laser diodes can be warmed. In some implementations, this is part of the multi-color image acquisition process.

At 2316, a determination as to the number of exposures can be made. In some implementations, this is part of the multi-color image acquisition process.

At 2318, the exposure(s) can be captured. In some implementations, this can be done using separate detectors for each of multiple color channels. For example, this is part of the multi-color image acquisition process.

At 2320, one or more fringes can be moved. In some implementations, SIM is used, and a grating or other SIM component can be moved. For example, the move can be done according to some periodicity. This can be part of the multi-color image acquisition process. This operation can be omitted in an implementation that does not involve SIM.

At 2322, a step and settle process can be initiated.

At 2324, a fine z-direction move can be made. This can be part of the step and settle process.

At 2326, a y-direction move can be made. This can involve individual operations of stepping (e.g., moving a cartridge or other sample carrier) and settling (e.g., allowing the carrier and its contents to come to rest so as to eliminate or minimize motion effects on a next capture).

At 2328, data transfer can be performed. In some implementations, one or more multiplexed fluorescent images can be transferred for analysis. For example, the analysis can be done for nucleotide identification in the sample.

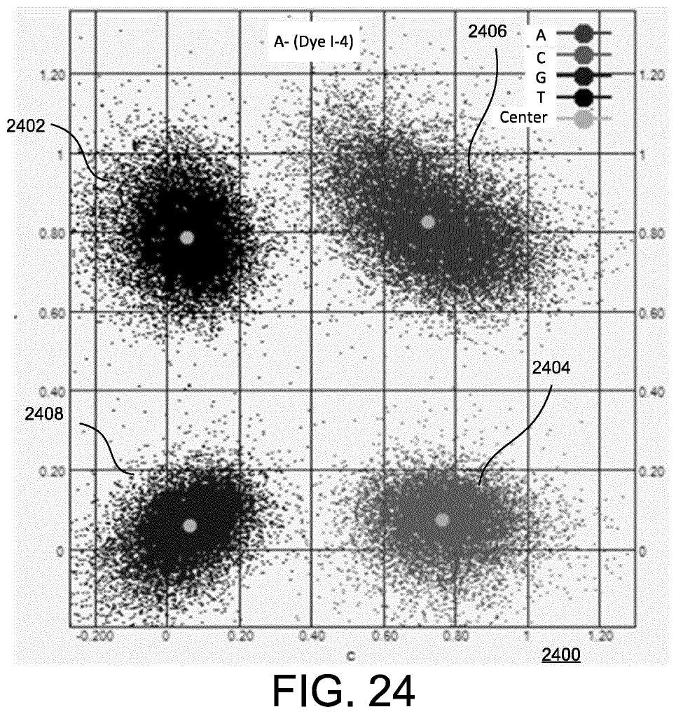

FIG. 24 is a scatterplot 2400 illustrating the usability of a fully functionalized A nucleotide labeled with dye I-4 described herein in a two-channel sequencing analysis. The amount of emitted light detected in the blue channel is indicated on the horizontal axis and the amount of emitted light detected in the green channel is indicated on the vertical axis. An emission 2402 corresponds to a substantive emission in the green channel and little or no emission in the blue channel. An emission 2404 corresponds to a substantive emission in the blue channel and little or no emission in the green channel. An emission 2406 corresponds to substantive emissions in both the blue and green channels. An emission 2408 corresponds to little or no emission in both the blue and green channels. Each of the emissions 2402-2408 can correspond to detection of a corresponding nucleotide. For example, the emission 2402 can correspond to detection of thymine. For example, the emission 2404 can correspond to detection of cytosine. For example, the emission 2406 can correspond to detection of adenine. For example, the emission 2408 can correspond to detection of guanine. In the simultaneous imaging of the present example, the emissions 2402-2408 are relatively separate from each other and show minimal or negligible crosstalk.

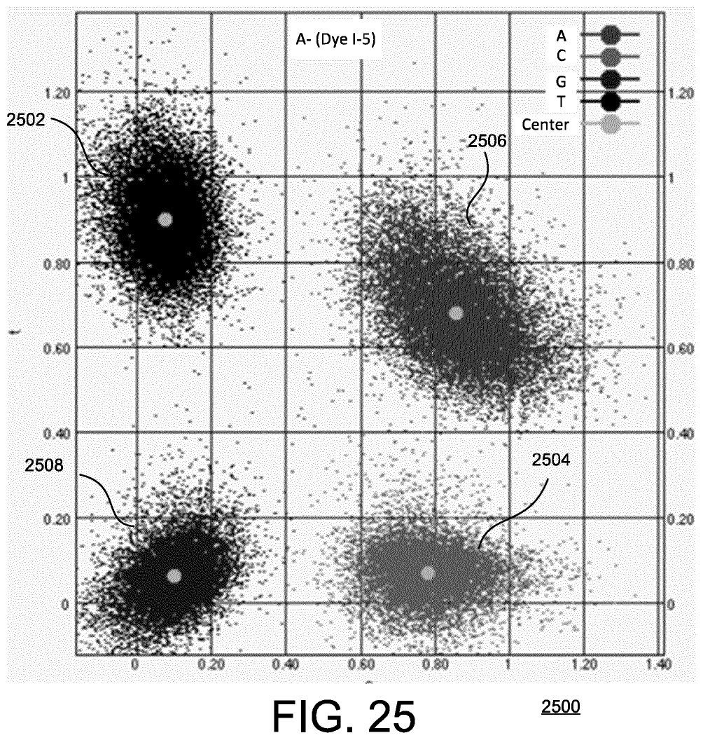

FIG. 25 is a scatterplot 2500 illustrating the usability of a fully functionalized A nucleotide labeled with dye I-5 described herein in a two-channel sequencing analysis. The amount of emitted light detected in the blue channel is indicated on the horizontal axis and the amount of emitted light detected in the green channel is indicated on the vertical axis. An emission 2502 corresponds to a substantive emission in the green channel and little or no emission in the blue channel. An emission 2504 corresponds to a substantive emission in the blue channel and little or no emission in the green channel. An emission 2506 corresponds to substantive emissions in both the blue and green channels. An emission 2508 corresponds to little or no emission in both the blue and green channels. Each of the emissions 2502-2508 can correspond to detection of a corresponding nucleotide. For example, the emission 2502 can correspond to detection of thymine. For example, the emission 2504 can correspond to detection of cytosine. For example, the emission 2506 can correspond to detection of adenine. For example, the emission 2508 can correspond to detection of guanine. In the simultaneous imaging of the present example, the emissions 2502-2508 are relatively separate from each other and show minimal or negligible crosstalk.

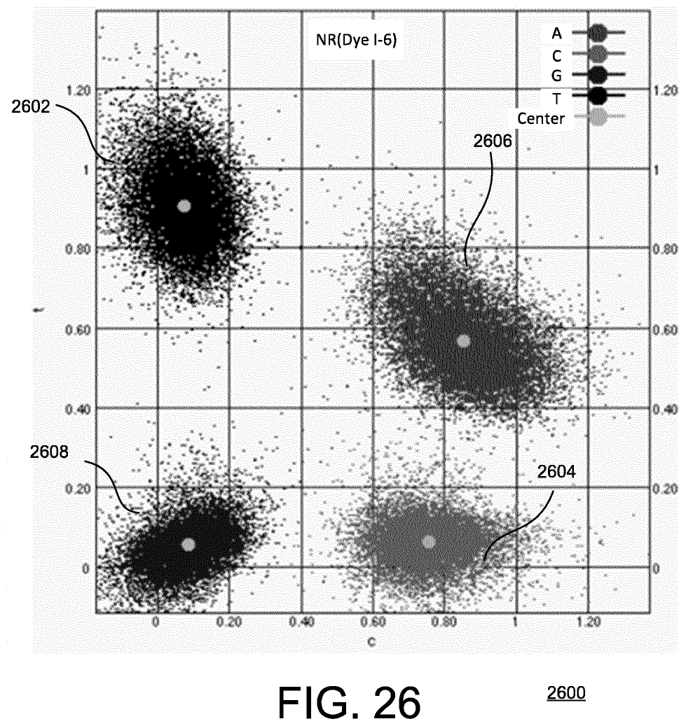

FIG. 26 is a scatterplot 2600 illustrating the usability of a fully functionalized A nucleotide labeled with dye I-6 described herein in a two-channel sequencing analysis. The amount of emitted light detected in the green channel is indicated on the vertical axis and the amount of emitted light detected in the blue channel is indicated on the horizontal axis. An emission 2602 corresponds to a substantive emission in the green channel and little or no emission in the blue channel. An emission 2604 corresponds to a substantive emission in the blue channel and little or no emission in the green channel. An emission 2606 corresponds to substantive emissions in both the blue and green channels. An emission 2608 corresponds to little or no emission in both the blue and green channels. Each of the emissions 2602-2608 can correspond to detection of a corresponding nucleotide. For example, the emission 2602 can correspond to detection of thymine. For example, the emission 2604 can correspond to detection of cytosine. For example, the emission 2606 can correspond to detection of adenine. For example, the emission 2608 can correspond to detection of guanine. In the simultaneous imaging of the present example, the emissions 2602-2608 are relatively separate from each other and show minimal or negligible crosstalk.

### III. Example Fluorescent Dyes

A. Example Blue Dyes

Fluorescent dye molecules with improved fluorescence properties such as suitable fluorescence intensity, shape, and wavelength maximum of fluorescence can improve the speed and accuracy of nucleic acid sequencing. Strong fluorescence signals are especially important when measurements are made in water-based biological buffers and at higher temperatures as the fluorescence intensities of most dyes are significantly lower under such conditions. Moreover, the nature of the base to which a dye is attached also affects the fluorescence maximum, fluorescence intensity, and others spectral dye properties. The sequence-specific interactions between the nucleobases and the fluorescent dyes can be tailored by specific design of the fluorescent dyes. Optimization of the structure of the fluorescent dyes can improve the efficiency of nucleotide incorporation, reduce the level of sequencing errors, and decrease the usage of reagents in, and therefore the costs of, nucleic acid sequencing.

Some optical and technical developments have already led to greatly improved image quality but were ultimately limited by poor optical resolution. Generally, optical resolution of light microscopy is limited to objects spaced at approximately half of the wavelength of the light used. In practical terms, then, only objects that are laying quite far apart (at least 200 to 350 nm) could be resolved by light microscopy. One way to improve image resolution and increase the number of resolvable objects per unit of surface area is to use excitation light of a shorter wavelength. For example, if light wavelength is shortened by Δλ˜100 nm with the same optics, resolution will be better (about Δ 50 nm/(about 15%)), less-distorted images will be recorded, and the density of objects on the recognizable area will be increased about 35%.

Certain nucleic acid sequencing methods employ laser light to excite and detect dye-labeled nucleotides. These instruments use longer wavelength light, such as red lasers, along with appropriate dyes that are excitable at 660 nm. To detect more densely packed nucleic acid sequencing clusters while maintaining useful resolution, a shorter wavelength blue light source (450-460 nm) may be used. In this case, optical resolution may be limited not by the emission wavelength of the longer wavelength red fluorescent dyes but rather by the emission of dyes excitable by the next longest wavelength light source, for example, by green laser light at 532 nm.

### Exocyclic Amine-Substituted Coumarin Dyes

Below are examples of exocyclic amine-substituted coumarin derivatives. The compounds may be useful as fluorescent labels, particularly for nucleotide labeling in nucleic acid sequencing applications. In some aspects, the dyes absorb light at short-wavelength light, optimally at a wavelength of 450-460 nm and are particularly advantageous in situations where blue wavelength excitation sources having a wavelength of 450-460 nm are used. Blue wavelength excitation allows detection and resolution of a higher density of features per unit area due to the shorter wavelength of fluorescence emission. When such dyes are used in conjugates with nucleotides, improvements can be seen in the length, intensity, accuracy, and quality of sequencing reads obtained during nucleic acid sequencing methods.

Some examples herein relate to exocyclic amine-substituted coumarin compounds particularly suitable for methods of fluorescence detection and sequencing by synthesis. Described herein are dyes and their derivatives of the structure of Formula (I), and salts thereof.

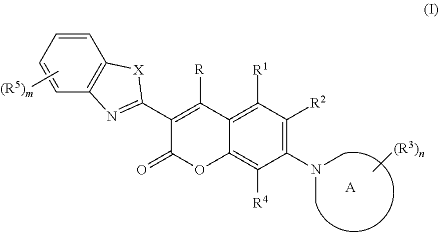

In some aspects, X is O. In some aspects, X is S. In some aspects, X is Se. In some aspects, X is NRⁿ, wherein Rⁿ is H, C₁₋₆ alkyl, or C₆₋₁₀ aryl, and in one aspect, Rⁿ is H. In some further implementations, when m is 1; R⁵ is —CO₂H; each of R, R¹, R², R⁴ is H; ring A is

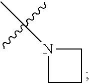 [SMILES: *N1CCC1]

then X is O, Se, or NRⁿ. In some further implementations, when n is 0; ring A is

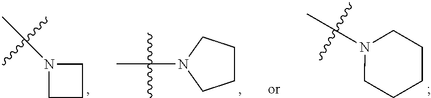 [SMILES: *N1CCC1 ; *N1CCCC1 ; *N1CCCCC1]

each of R, R¹, R², R⁴ is H; X is O; then m is 1, 2, 3, or 4. In some aspects, when n is 0, then m is 1, 2, 3, or 4 and at least one R⁵ is —CO₂H. In some other aspects, when n is 1 and R³ is —CO₂H, then m is 0 or R⁵ is not —CO₂H.

In some aspects, R is H, halo, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl. In one aspect, R is H. In another aspect, R is halo. In some aspects, R is optionally substituted C₁₋₆ alkyl. In some aspects, R is —CO₂H. In some aspects, R is —SO₃H. In some aspects, R is —SO₂NR^{a}R^{b}, wherein R^{a} and R^{b} is independently H or optionally substituted C₁₋₆ alkyl. In one aspect, R is —SO₂NH₂ In some aspect, R is not-CN.

In some aspects, R¹ is H. In some aspects, R¹ is halo. In some aspects, R¹ is —CN.

In some aspects, R¹ is C₁₋₆ alkyl. In some aspects, R¹ is —SO₂NR^{a}R^{b}, wherein R^{a} and R^{b} is independently H or optionally substituted C₁₋₆ alkyl. In one aspect, R¹ is —SO₂NH₂. In some aspect, R¹ is not —CN.

In some aspects, R² is H. In some aspects, R² is halo. In some aspect, R² is —SO₃H. In some aspects, R² is optionally substituted alkyl, for example C₁₋₆ alkyl. In some further implementations, R² is C₁₋₄ alkyl optionally substituted with —CO₂H or —SO₃H.

In some aspects, R⁴ is H. In some aspects, R⁴ is —SO₃H. In some aspects, R⁴ is optionally substituted alkyl, for example C₁₋₆ alkyl. In some further implementations, R⁴ is C₁₋₄ alkyl optionally substituted with —CO₂H or —SO₃H.

In some aspects, ring A is a 3 to 7 membered single heterocyclic ring. In some further implementations, the 3 to 7 membered single heterocyclic ring contains one nitrogen atom. In some aspects, ring A

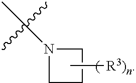

In one such implementation, ring A is

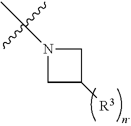

In some aspects, ring A is

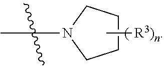

In one such implementation, ring A is

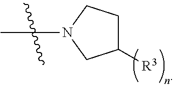

In some aspects, ring A is

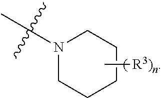

In one such implementation, ring A is

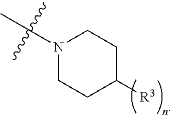

In some aspects of the ring A described herein, n is 0. In some aspects of the ring A described herein, n is 1. In some aspects of the ring A described herein, n is 2 or 3. In some aspects, each R³ is independently —CO₂H, —SO₃H, C₁₋₄ alkyl optionally substituted with —CO₂H or —SO₃H, —(CH₂)ₚ—CO₂R^{c}, or optionally substituted C₁₋₆ alkyl. In some aspects, R³ is methyl, ethyl, propyl, isopropyl, butyl, isobutyl, sec-butyl, tert-butyl, pentyl, or hexyl. In other aspects, R³ is substituted C₁₋₄ alkyl. In some aspects, R³ is C₁₋₄ alkyl or C₂₋₆ alkyl substituted with —CO₂H or —SO₃H. In some further implementations, n is 1 and R³ is —CO₂H or —(CH₂)ₚ—CO₂R^{c}. In some further implementations, R is H or C₁₋₄ alkyl.

The benzene ring of the

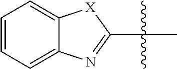

moiety of Formula (I) is optionally substituted in any one, two, three, or four positions by a substituent shown as R⁵. Where m is zero, the benzene ring is unsubstituted. Where m is greater than 1, each R⁵ may be the same or different. In some aspects, m is 0. In other aspects, m is 1. In other aspects, m is 2. In some aspects, m is 1, 2, or 3, and each R⁵ is independently halo, —CN, —CO₂R, amino, —OH, —SO₃H, —SO₂NR^{a}R^{b} or optionally substituted C₁₋₆ alkyl, where R^{f} is H or C₁₋₄ alkyl. In some further implementations, R⁵ is —CO₂H, —SO₃H, —SO₂NH₂, or C₁₋₆ alkyl substituted with —CO₂H, —SO₃H, or —SO₂NH₂. In some further implementations, R⁵ is —(CH₂)ₓCOOH where x is 2, 3, 4, 5 or 6. In some implementations, when each of R, R¹, R², R⁴ is H; n is 0; m is 1; then

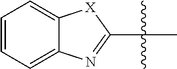

is substituted at the following position:

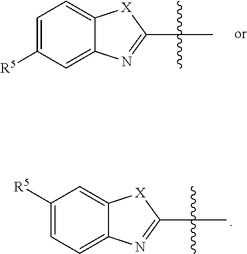

In one implementation, R⁵ is —CO₂H.

Particular examples of a compound of Formula (I) include where X is O, S or NH; each R, R¹, R², and R⁴ is H; ring A is

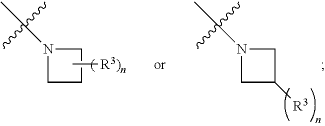

or n is 0 or 1; R is —CO₂H or —(CH₂)ₚ—CO₂R^{c}; p is 1, 2, 3, or 4; R^{c} is H or C₁₋₆ alkyl; m is 0 or 1; and R⁵ is halo, —CO₂R, —SO₃H, —SO₂NR^{a}R^{b}, or C₁₋₆ alkyl substituted with —SO₃H or —SO₂NR^{a}R^{b}. In some implementations, at least one or both of R^{a} and R^{b} is H or C₁₋₆ alkyl. In some further implementations, R^{f} is H or C₁₋₄ alkyl. In some further implementations, when m is 0, then n is 1; or when n is 0, then m is 1. In one implementation, both m and n are 1.

Particular examples of a compound of Formula (I) include where X is O, S or NH; each R, R¹, R², and R⁴ is H; ring A is

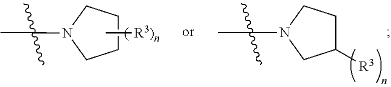

n is 0 or 1; R³ is —CO₂H or —(CH₂)ₚ—CO₂R^{c}; p is 1, 2, 3, or 4; R^{c} is H or C₁₋₆ alkyl; m is 0 or 1; and R⁵ is halo, —CO₂R, —SO₃H, —SO₂NR^{a}R^{b}, or C₁₋₆ alkyl substituted with —SO₃H or —SO₂NR^{a}R^{b}. In some implementations, at least one or both of R^{a} and R^{b} is H or C₁₋₆ alkyl. In some further implementations, R^{f} is H or C₁₋₄ alkyl. In some further implementations, when m is 0, then n is 1; or when n is 0, then m is 1. In one implementation, both m and n are 1.

Particular examples of a compound of Formula (I) include where X is O, S or NH; each R, R¹, R², and R⁴ is H; ring A is

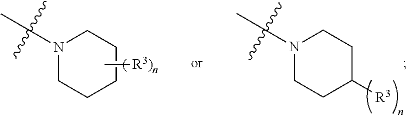

n is 0 or 1; R³ is-CO₂H or —(CH₂)ₚ—CO₂R^{c}; p is 1, 2, 3, or 4; R^{c} is H or C₁₋₆ alkyl; m is 0 or 1; and R⁵ is halo, —CO₂^{f}, —SO₃H, —SO₂NR^{a}R^{b}, or C₁₋₆ alkyl substituted with —SO₃H or —SO₂NR^{a}R^{b}. In some implementations, at least one or both of R^{a} and R^{b} is H or C₁₋₆ alkyl. In some further implementations, R^{f} is H or C₁₋₄ alkyl. In some further implementations, when m is 0, then n is 1; or when n is 0, then m is 1. In one implementation, both m and n are 1.

Specific examples of exocyclic amine-substituted coumarin dyes include:

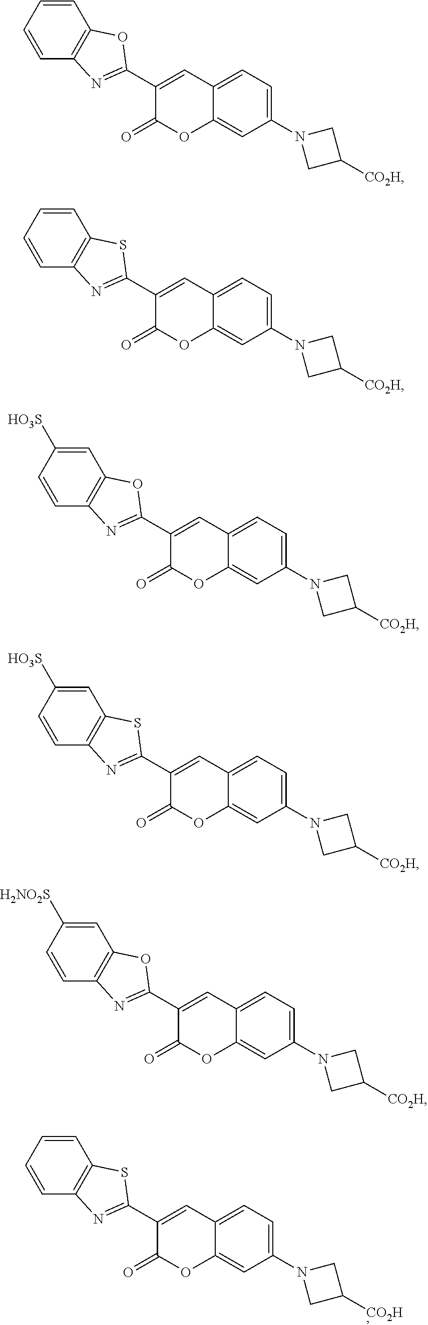 [SMILES: O=C(O)C1CN(c2ccc3cc(-c4nc5ccccc5o4)c(=O)oc3c2)C1 ; O=C(O)C1CN(c2ccc3cc(-c4nc5ccccc5s4)c(=O)oc3c2)C1 ; O=C(O)C1CN(c2ccc3cc(-c4nc5ccc(S(=O)(=O)O)cc5o4)c(=O)oc3c2)C1 ; O=C(O)C1CN(c2ccc3cc(-c4nc5ccc(S(=O)(=O)O)cc5s4)c(=O)oc3c2)C1 ; NS(=O)(=O)c1ccc2nc(-c3cc4ccc(N5CC(C(=O)O)C5)cc4oc3=O)oc2c1 ; O=C(O)C1CN(c2ccc3cc(-c4nc5ccccc5s4)c(=O)oc3c2)C1]

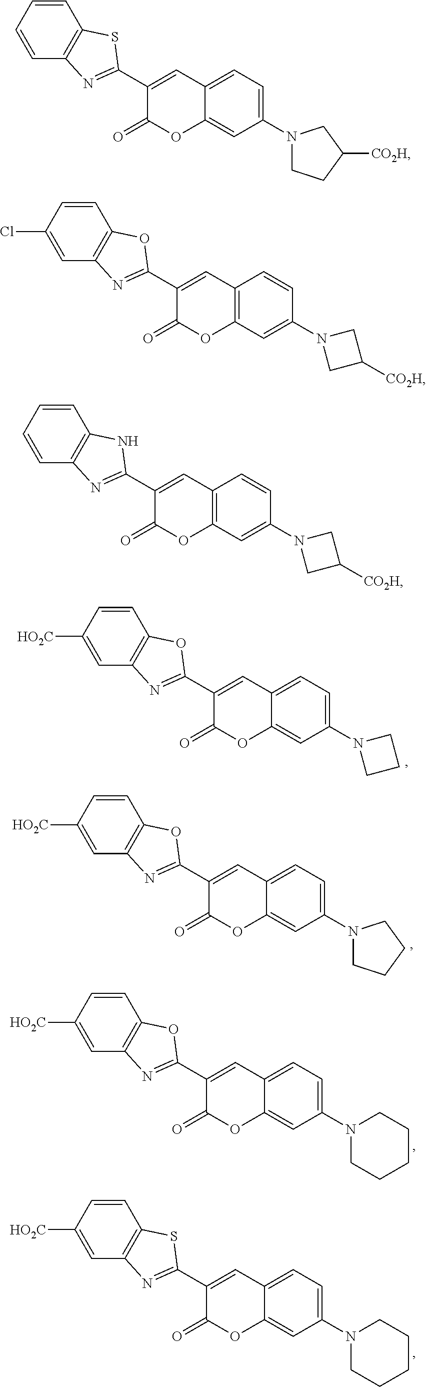

and salts thereof.

A particularly useful compound is a nucleotide or oligonucleotide labeled with a dye as described herein. The labeled nucleotide or oligonucleotide may be attached to the dye compound disclosed herein via a carboxy or an alkyl-carboxy group to form an amide or alkyl-amide. For example, the dye compound disclosed herein is attached the nucleotide or oligonucleotide via R³ or R⁵ of Formula (I). In some implementations, R³ of Formula (I) is —CO₂H or —(CH₂)ₚ—CO₂H and the attachment forms an amide using the —CO₂H group. In some implementations, R⁵ of Formula (I) is —CO₂H and the attachment forms an amide using the —CO₂H group. The labeled nucleotide or oligonucleotide may have the label attached to the C5 position of a pyrimidine base or the C7 position of a 7-deaza purine base through a linker moiety.

The labeled nucleotide or oligonucleotide may also have a blocking group covalently attached to the ribose or deoxyribose sugar of the nucleotide. The blocking group may be attached at any position on the ribose or deoxyribose sugar. In particular implementations, the blocking group is at the 3′ OH position of the ribose or deoxyribose sugar of the nucleotide.

### Tertiary Amine-Substituted Coumarin Dyes

Also disclosed herein are tertiary amine substituted coumarin compounds particularly suitable for methods of fluorescence detection and sequencing by synthesis. Implementations of the tertiary amine substituted coumarin dyes have excellent water solubility while exhibiting strong fluorescence in water or polar solvents/buffers, thus are suitable for nucleotide labeling and sequencing application in aqueous environment. Implementations described herein relate to dyes and their derivatives of the structure of Formula (II), and salts thereof.

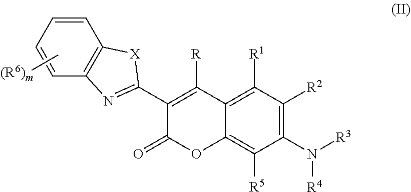

In some aspects, X is O. In some aspects, X is S. In some aspects, X is Se. In some aspects, X is NRⁿ, wherein Rⁿ is H, C₁₋₆ alkyl, or C₆₋₁₀ aryl, and in one aspect, Rⁿ is H or phenyl. In some further implementations, when m is 1, 2, 3 or 4 and one of R⁶ is —CO₂H; each of R, R¹, R², R⁵ is H; then each of R³ and R⁴ is independently C₁₋₆ alkyl, —(CH₂)ₚ—CO₂R^{c}, —(CH₂)_{q}—C(O)NR^{d}R^{e}, —(CH₂)ₙ—SO₃H, —(CH₂)ₜ—SO₂NR^{a}R^{b}, where R^{c} is optionally substituted C₁₋₆ alkyl, optionally substituted carbocyclyl, optionally substituted heterocyclyl, optionally substituted aryl, or optionally substituted heteroaryl. In other words, when R⁶ is —CO₂H, neither R³ or R⁴ comprises a —CO₂H moiety. In some other implementations, when m is 0 or R⁶ is not —CO₂H; each of R, R¹, R², R⁵ is H; then at least one of R³ or R⁴ comprises a —CO₂H.

In some aspects, R is H, halo, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, or optionally substituted heteroaryl. In one aspect, R is H. In another aspect, R is halo. In some aspects, R is optionally substituted C₁₋₆ alkyl. In some aspects, R is —CO₂H. In some aspects, R is —SO₃H. In some aspects, R is —SO₂NR^{a}R^{b}, wherein R^{a} and R^{b} is independently H or optionally substituted C₁₋₆ alkyl. In one aspect, R is —SO₂NH₂ In some aspect, R is not —CN.

In some aspects, R¹ is H. In some aspects, R¹ is halo. In some aspects, R¹ is —CN.

In some aspects, R¹ is C₁₋₆ alkyl. In some aspects, R¹ is —SO₂NR^{a}R^{b}, wherein R^{a} and R^{b} is independently H or optionally substituted C₁₋₆ alkyl. In one aspect, R¹ is —SO₂NH₂ In some aspect, R¹ is not —CN.

In some aspects, R² is H. In some aspects, R² is halo. In some aspect, R² is —SO₃H. In some aspects, R² is optionally substituted alkyl, for example C₁₋₆ alkyl. In some further implementations, R² is C₁₋₄ alkyl optionally substituted with —CO₂H or —SO₃H.

In some aspects, R⁵ is H. In some aspects, R⁵ is halo. In some aspect, R⁵ is —SO₃H. In some aspects, R² is optionally substituted alkyl, for example C₁₋₆ alkyl. In some further implementations, R⁵ is C₁₋₄ alkyl optionally substituted with —CO₂H or —SO₃H.

In some aspects, R³ is —(CH₂)ₚ—CO₂R^{c}. In further implementations, p is 2, 3, 4, or 5. R^{c} is H or C₁₋₆ alkyl, for example, methyl, ethyl, isopropyl or t-butyl. In some aspects, R³ is C₁₋₆ alkyl.

In some aspects, R⁴ is —(CH₂)ₙ—SO₃H. In further implementations, n is 2, 3, 4, or 5. In some aspects, R⁴ is C₁₋₆ alkyl.

In some aspects, at least one of R³ and R⁴ is C₁₋₆ alkyl. In some aspects, both R³ and R⁴ are C₁₋₆ alkyl. In some aspects, when R³ is —(CH₂)ₚ—CO₂R^{c}, then R⁴ is —(CH₂)ₙ—SO₃H. In some aspects, both R³ and R⁴ are —(CH₂)ₚ—CO₂R^{c}.

The benzene ring of the

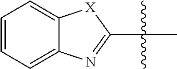

moiety of Formula (II) is optionally substituted in any one, two, three, or four positions by a substituent shown as R⁶. Where m is zero, the benzene ring is unsubstituted. Where m is greater than 1, each R⁶ may be the same or different. In some aspects, m is 0. In other aspects, m is 1. In other aspects, m is 2. In some aspects, m is 1, 2, or 3, and each R⁶ is independently halo, —CN, —CO₂R, amino, —OH, —SO₃H, —SO₂NR^{a}R^{b} or optionally substituted C₁₋₆ alkyl, where R^{f} is H or C₁₋₄ alkyl. In some further implementations, R⁶ is —CO₂H, —SO₃H, —SO₂NH₂, or C₁₋₆ alkyl substituted with —CO₂H, —SO₃H, or —SO₂NH₂. In some further implementations, R⁶ is —(CH₂)ₓCOOH where x is 2, 3, 4, 5 or 6. In some implementations, when each of R, R¹, R², R⁵ is H; R³ and R⁴ is independently C₁₋₆ alkyl, —(CH₂)ₚ—CO₂R^{c}, —(CH₂)_{q}—C(O)NR^{d}R^{e}, —(CH₂)ₙ—SO₃H, —(CH₂)ₜ—SO₂NR^{a}R^{b}, where R^{c} is optionally substituted C₁₋₆ alkyl, optionally substituted carbocyclyl, optionally substituted heterocyclyl, optionally substituted aryl, or optionally substituted heteroaryl (i.e., neither R³ and R⁴ comprises —CO₂H); m is 1; then

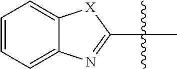

is at substituted at the following position:

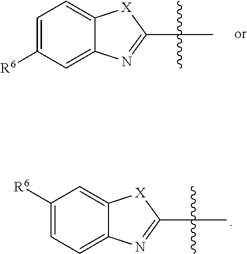

In one implementation, R⁶ is —CO₂H. In another implementation, R⁶ is halo, such —Cl. In yet another implementation, R⁶ is —SO₂NR^{a}R^{b} where at least one or both of R^{a} and R^{b} is H or C₁₋₆ alkyl.

Particular examples of a compound of Formula (II) include where X is O, S or NH; each R, R¹, R², and R⁵ is H; R³ is —(CH₂)ₚ—CO₂R^{c} or C₁₋₆ alkyl; R⁴ is C₁₋₆ alkyl or —(CH₂)ₙ—SO₃H; m is 0 or 1; and R⁶ is —SO₃H, —SO₂NR^{a}R^{b}, halo, —CO₂H, or C₁₋₆ alkyl substituted with —CO₂H, —SO₃H or —SO₂NR^{a}R. In some implementations, at least one or both of R^{a} and R^{b} is H or C₁₋₆ alkyl. In some further implementations, when R³ is —(CH₂)ₚ—CO₂R^{c}, then R⁴ is —(CH₂)ₙ—SO₃H or C₁₋₆alkyl. In some further implementations, both R³ and R⁴ are C₁₋₆ alkyl. When m is 1,

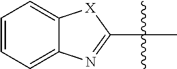

is at substituted at the following position:

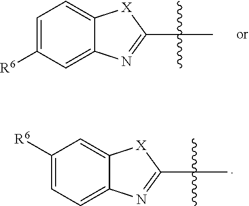

In one implementation, R⁶ is —CO₂H. In another implementation, R⁶ is halo, such as chloro. In yet another implementation, R⁶ is —SO₂NR^{a}R^{b} where at least one or both of R^{a} and R^{b} is H or C₁₋₆ alkyl.

Specific examples of the tertiary amine-substituted coumarin dyes include:

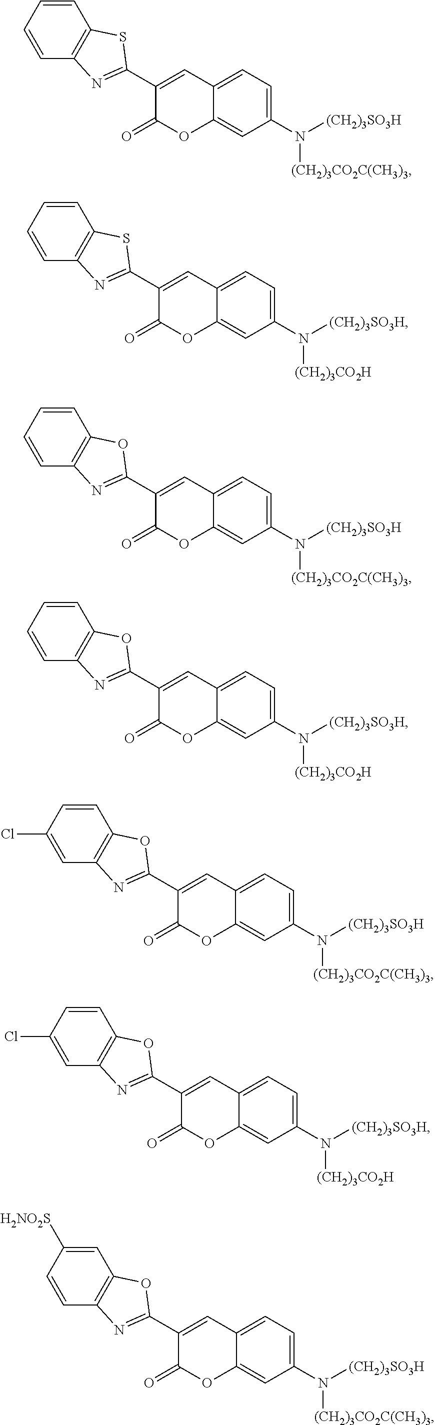

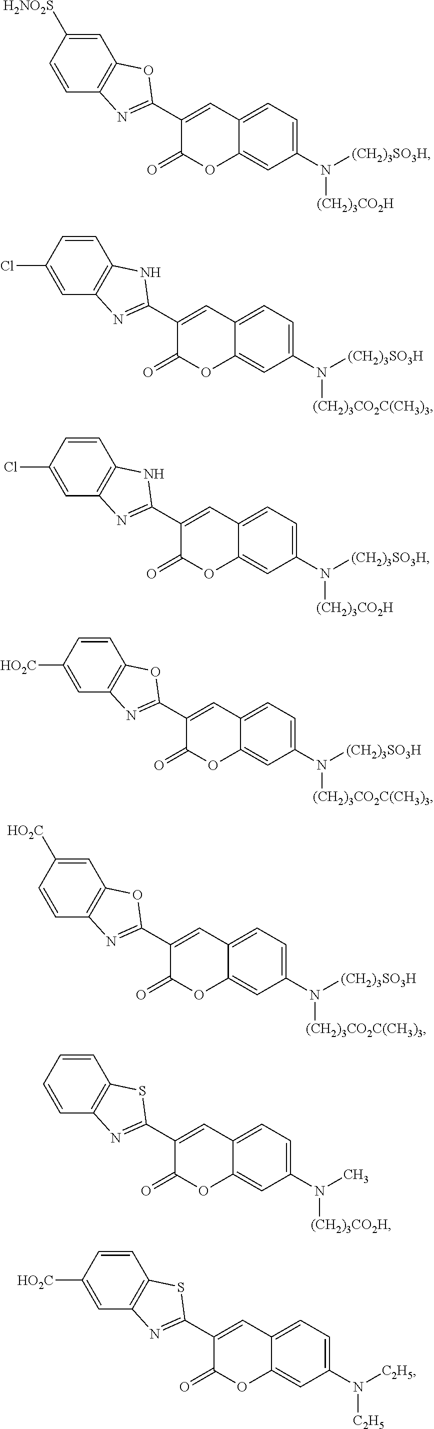

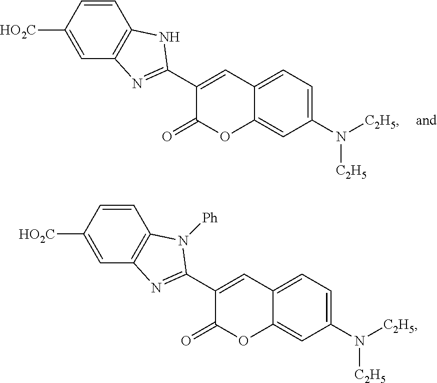 [SMILES: CCN(CC)c1ccc2cc(-c3nc4cc(C(=O)O)ccc4[nH]3)c(=O)oc2c1 ; CCN(CC)c1ccc2cc(-c3nc4cc(C(=O)O)ccc4n3-c3ccccc3)c(=O)oc2c1]

and salts thereof.

Additional coumarin dyes with secondary amine substitution include:

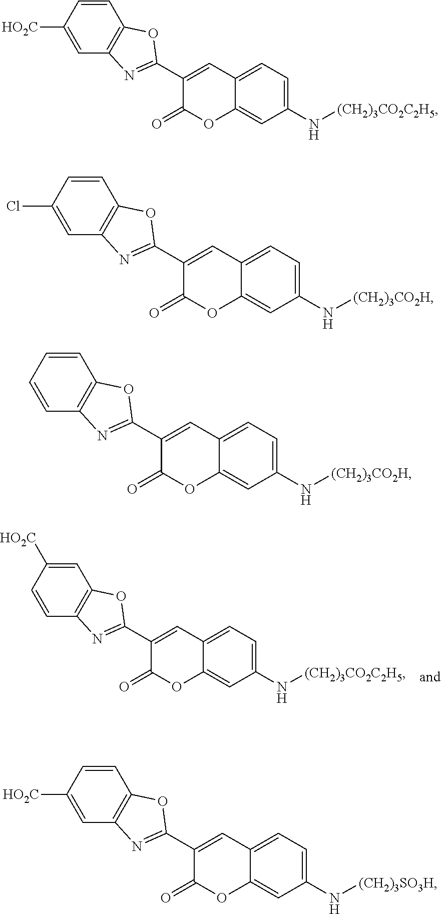

and salts thereof.

A particularly useful compound is a nucleotide or oligonucleotide labeled with a dye as described herein. The labeled nucleotide or oligonucleotide may be attached to the dye compound disclosed herein via a carboxy or an alkyl-carboxy group to form an amide or alkyl-amide. For example, the dye compound disclosed herein is attached the nucleotide or oligonucleotide via R³, R⁴ or R⁶ of Formula (II). In some implementations, R³ or R⁴ of Formula (II) is —CO₂H or —(CH₂)ₚ—CO₂H and the attachment forms an amide using the —CO₂H group. In some implementations, R⁶ of Formula (II) is —CO₂H and the attachment forms an amide using the —CO₂H group. The labeled nucleotide or oligonucleotide may have the label attached to the C5 position of a pyrimidine base or the C7 position of a 7-deaza purine base through a linker moiety.

The labeled nucleotide or oligonucleotide may also have a blocking group covalently attached to the ribose or deoxyribose sugar of the nucleotide. The blocking group may be attached at any position on the ribose or deoxyribose sugar. In particular implementations, the blocking group is at the 3′ OH position of the ribose or deoxyribose sugar of the nucleotide.

The compounds disclosed herein typically absorb light in the region below 500 nm. The compounds or nucleotides that are set forth herein may be used to detect, measure, or identify a biological system (including, for example, processes or components thereof). Some techniques that can employ the compounds or nucleotides include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two lasers operating at different wavelengths.

Disclosed herein are methods of synthesizing compounds of the disclosure. Dyes according to the present disclosure may be synthesized from a variety of different suitable starting materials. Methods for preparing coumarin dyes are well known in the art.

Unless defined otherwise, all technical and scientific terms used herein have the same meaning as is commonly understood by one of ordinary skill in the art. As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁₋₄alkyl” or similar designations. By way of example only, “C₁₋₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂₋₆alkenyl” or similar designations. By way of example only, “C₂₋₆alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂₋₆alkynyl” or similar designations. By way of example only, “C₂₋₆alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 6 carbon atoms. The heteroalkyl group may be designated as “C₁₋₆ heteroalkyl” or similar designations. The heteroalkyl group may contain one or more heteroatoms. By way of example only, “C₄₋₆ heteroalkyl” indicates that there are four to six carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some implementations, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some implementations, the heteroaryl group has 5 to ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfino” group refers to a “—S(═O)OH” group.

A “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, and oxo (═O). Wherever a group is described as “optionally substituted” that group can be substituted with the above substituents.

In some implementations, substituted alkyl, alkenyl, or alkynyl groups are substituted with one or more substituents selected from the group consisting of halo, —CN, SO₃⁻, —SO₃H, —SR^{A}, —OR^{A}, —NR^{B}R^{C}, oxo, —CONR^{B}R^{C}, —SO₂NR^{B}R^{C}, —COOH, and —COOR^{B}, where R^{A}, R^{B} and R^{C} are each independently selected from H, alkyl, substituted alkyl, alkenyl, substituted alkenyl, alkynyl, substituted alkynyl, aryl, and substituted aryl.

Compounds described herein can be represented as several mesomeric forms. Where a single structure is drawn, any of the relevant mesomeric forms are intended. The coumarin compounds described herein are represented by a single structure but can equally be shown as any of the related mesomeric forms. Some mesomeric structures are shown below for Formula (I):

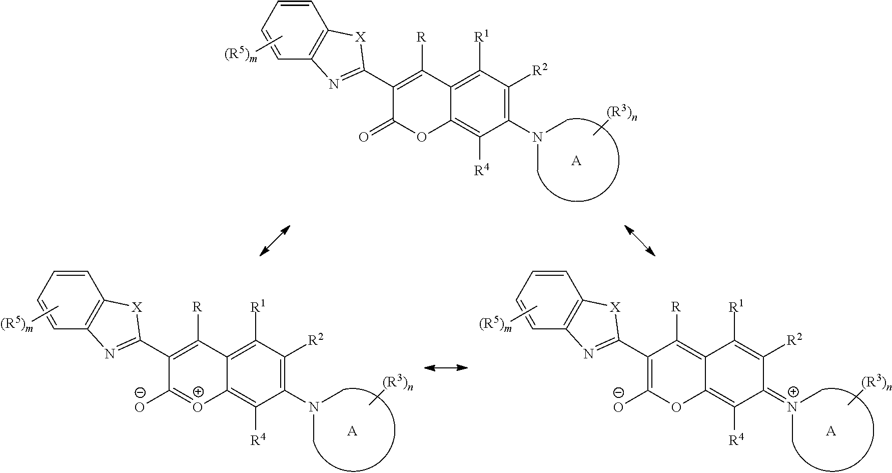

Some mesomeric structures are shown below for Formula (II).

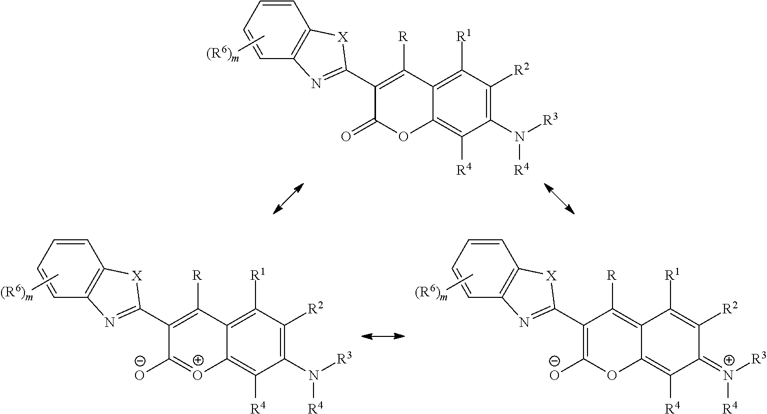

In each instance where a single mesomeric form of a compound described herein is shown, the alternative mesomeric forms are equally contemplated.

As understood by one of ordinary skill in the art, a compound described herein may exist in ionized form, e.g., —CO₂⁻ or —SO₃⁻. If a compound contains a positively or negatively charged substituent group, for example, SO₃⁻, it may also contain a negatively or positively charged counterion such that the compound as a whole is neutral. In other aspects, the compound may exist in a salt form, where the counterion is provided by a conjugate acid or base.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two “adjacent” R groups are said to form a ring “together with the atom to which they are attached,” it is meant that the collective unit of the atoms, intervening bonds, and the two R groups are the recited ring. For example, when the following substructure is present:

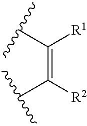

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

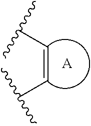

where A is an aryl ring or a carbocyclyl containing the depicted double bond.

### Labeled Nucleotides

According to an aspect of this disclosure, there are provided dye compounds suitable for attachment to substrate moieties, particularly comprising linker groups to enable attachment to substrate moieties. Substrate moieties can be virtually any molecule or substance to which the dyes of the disclosure can be conjugated, and, by way of non-limiting example, may include nucleosides, nucleotides, polynucleotides, carbohydrates, ligands, particles, solid surfaces, organic and inorganic polymers, chromosomes, nuclei, living cells, and combinations or assemblages thereof. The dyes can be conjugated by an optional linker by a variety of means including hydrophobic attraction, ionic attraction, and covalent attachment. In some aspects, the dyes are conjugated to the substrate by covalent attachment. More particularly, the covalent attachment is by means of a linker group. In some instances, such labeled nucleotides are also referred to as “modified nucleotides.”

The present disclosure further provides conjugates of nucleosides and nucleotides labeled with one or more of the dyes set forth herein (modified nucleotides). Labeled nucleosides and nucleotides are useful for labeling polynucleotides formed by enzymatic synthesis, such as, by way of non-limiting example, in PCR amplification, isothermal amplification, solid phase amplification, polynucleotide sequencing (e.g., solid phase sequencing), nick translation reactions and the like.

The attachment to the biomolecules may be via the R, R¹, R², R³, R⁴, R⁵, or X position of the compound of Formula (I). In some aspects, the connection is via the R³ or R⁵ group of Formula (I). The attachment to the biomolecules may be via the R, R¹, R², R³, R⁴, R⁵, R⁶ or X position of the compound of Formula (II). In some aspects, the connection is via the R³, R⁴ or R⁶ group of Formula (II). In some implementations, the substituent group is a carboxyl or substituted alkyl, for example, alkyl substituted with —CO₂H or an activated form of carboxyl group, for example, an amide or ester, which may be used for attachment to the amino or hydroxyl group of the biomolecules. The term “activated ester” as used herein, refers to a carboxyl group derivative which is capable of reacting in mild conditions, for example, with a compound containing an amino group. Non-limiting examples of activated esters include but not limited to p-nitrophenyl, pentafluorophenyl and succinimido esters.

In some implementations, the dye compounds may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a linker moiety. The labeled nucleotide or oligonucleotide may also have a 3-OH blocking group covalently attached to the ribose or deoxyribose sugar of the nucleotide.

A particular useful application of the fluorescent dyes as described herein is for labeling biomolecules, for example, nucleotides or oligonucleotides. Some implementations of the present application are directed to a nucleotide or oligonucleotide labeled with the fluorescent compounds as described herein.

### Linkers

The dye compounds as disclosed herein may include a reactive linker group at one of the substituent positions for covalent attachment of the compound to a substrate or another molecule. Reactive linking groups are moieties capable of forming a bond (e.g., a covalent or non-covalent bond), in particular a covalent bond. In a particular implementation, the linker may be a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Pub. No. WO 2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Pub. No. WO 2004/018493 (herein incorporated by reference) such as those that include moieties of the formulae:

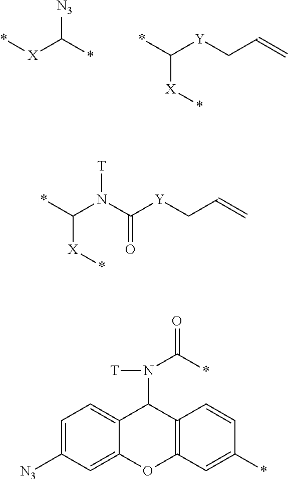

(wherein X is selected from the group comprising O, S, NH and NQ wherein Q is a C1-10 substituted or unsubstituted alkyl group, Y is selected from the group comprising 0, S, NH and N(allyl), T is hydrogen or a C₁-C₁₀ substituted or unsubstituted alkyl group and * indicates where the moiety is connected to the remainder of the nucleotide or nucleoside). In some aspects, the linkers connect the bases of nucleotides to labels such as, for example, the dye compounds described herein.

Additional examples of linkers include those disclosed in U.S. Pub. No. 2016/0040225 (herein incorporated by reference), such as those include moieties of the formulae:

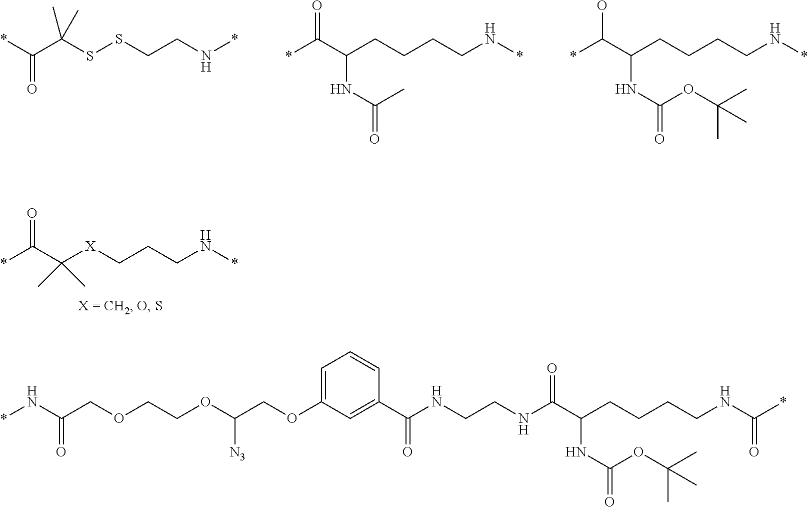

(wherein * indicates where the moiety is connected to the remainder of the nucleotide or nucleoside). The linker moieties illustrated herein may comprise the whole or partial linker structure between the nucleotides/nucleosides and the labels.

In particular implementations, the length of the linker between a fluorescent dye (fluorophore) and a guanine base can be altered, for example, by introducing a polyethylene glycol spacer group, thereby increasing the fluorescence intensity compared to the same fluorophore attached to the guanine base through other linkages known in the art. Some linkers and their properties are set forth in PCT Pub. No. WO 2007/020457 (herein incorporated by reference). The design of linkers, and especially their increased length, can allow improvements in the brightness of fluorophores attached to the guanine bases of guanosine nucleotides when incorporated into polynucleotides such as DNA. Thus, when the dye is for use in any method of analysis which requires detection of a fluorescent dye label attached to a guanine-containing nucleotide, it is advantageous if the linker comprises a spacer group of formula —((CH₂)₂O)ₙ—, wherein n is an integer between 2 and 50, as described in PCT Pub. No. WO 2007/020457.

Nucleosides and nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxyl group that is present in ribose. The nitrogenous base is a derivative of purine or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxyl group attached to the C-3 or C-5 of the sugar. Nucleotides are usually mono, di- or triphosphates.

A “nucleoside” is structurally similar to a nucleotide but is missing the phosphate moieties. An example of a nucleoside analog would be one in which the label is linked to the base and there is no phosphate group attached to the sugar molecule.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular implementations, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidate linkages and the like.

A dye may be attached to any position on the nucleotide base, for example, through a linker. In particular implementations, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleoside or nucleotide.

In particular implementations, the labeled nucleoside or nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

Nucleosides or nucleotides labeled with the dyes described herein may have the formula:

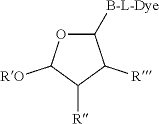

where Dye is a dye compound; B is a nucleobase, such as, for example uracil, thymine, cytosine, adenine, guanine and the like; L is an optional linker group which may or may not be present; R′ can be H, monophosphate, diphosphate, triphosphate, thiophosphate, a phosphate ester analog, —O— attached to a reactive phosphorous containing group, or —O— protected by a blocking group; R″ can be H, OH, a phosphoramidite, or a 3′-OH blocking group, and R′″ is H or OH. Where R″ is phosphoramidite, R′ is an acid-cleavable hydroxyl protecting group which allows subsequent monomer coupling under automated synthesis conditions.

In a particular implementation, the blocking group is separate and independent of the dye compound, i.e., not attached to it. Alternatively, the dye may comprise all or part of the 3′-OH blocking group. Thus R″ can be a 3′-OH blocking group which may or may not comprise the dye compound.

In yet another alternative implementation, there is no blocking group on the 3′ carbon of the pentose sugar and the dye (or dye and linker construct) attached to the base, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

In still yet another alternative implementation, the blocking group is present on the 2′ or 4′ carbon of the pentose sugar and can be of a size or structure sufficient to act as a block to the incorporation of a further nucleotide.

The use of a blocking group allows polymerization to be controlled, such as by stopping extension when a modified nucleotide is incorporated. If the blocking effect is reversible, for example, by way of non-limiting example by changing chemical conditions or by removal of a chemical block, extension can be stopped at certain points and then allowed to continue.

In another particular implementation, a 3′-OH blocking group will comprise a moiety disclosed in PCT Pub. No. WO 2004/018497 and WO 2014/139596, the disclosures of each is incorporated herein by reference in its entirety. For example the blocking group may be azidomethyl (—CH₂N₃) or substituted azidomethyl (e.g., —CH(CHF₂)N₃ or CH(CH₂F)N₃), or allyl.

In a particular implementation, the linker (between dye and nucleotide) and blocking group are both present and are separate moieties. In particular implementations, the linker and blocking group are both cleavable under substantially similar conditions. Thus, deprotection and deblocking processes may be more efficient because only a single treatment will be required to remove both the dye compound and the blocking group. However, in some implementations a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting labeled nucleotides as described herein include:

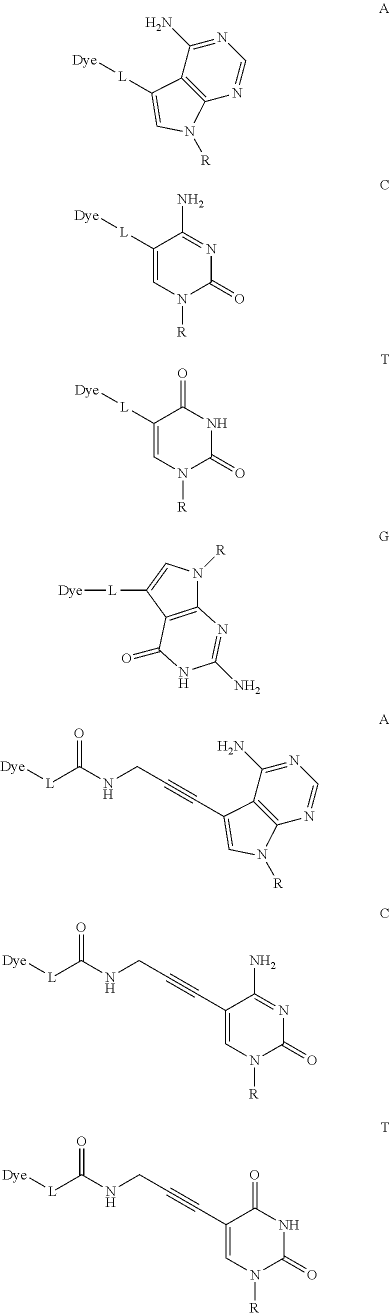

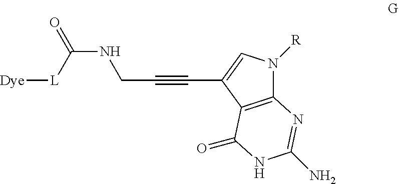

wherein L represents a linker and R represents a sugar residue as described above.

In some implementations, non-limiting fluorescent dye conjugates are shown below:

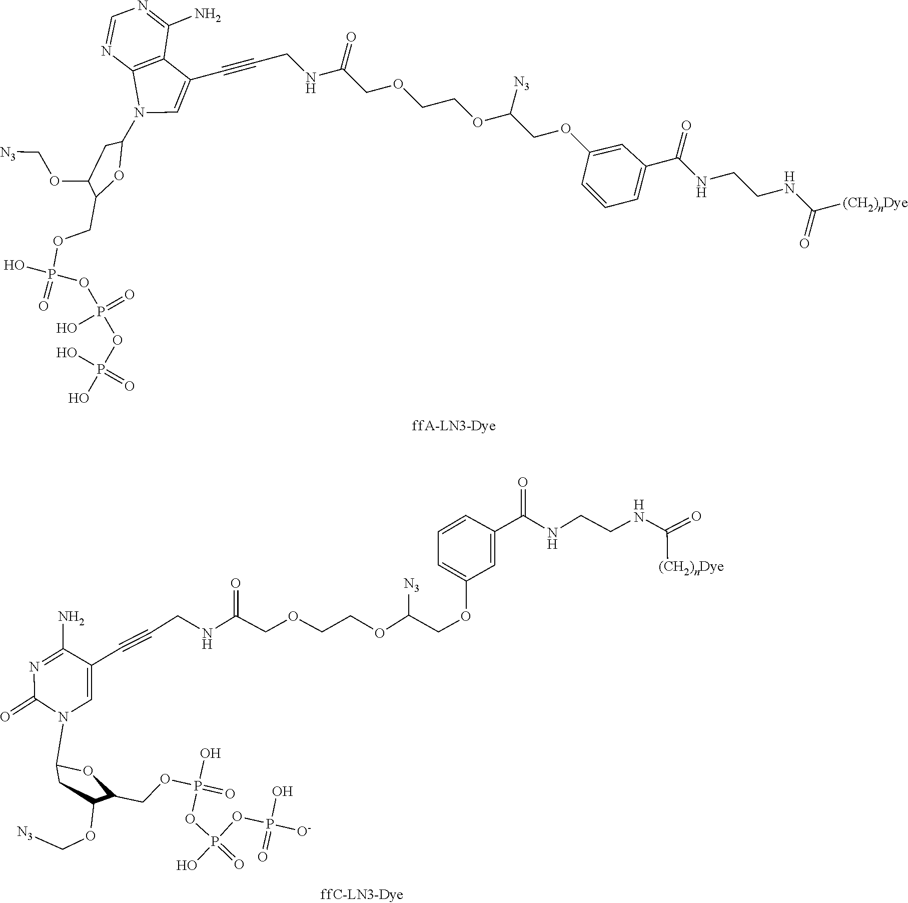

### Kits

The present disclosure also provides kits including modified nucleosides and/or nucleotides labeled with dyes. Such kits will generally include at least one modified nucleotide or nucleoside labeled with a dye set forth herein together with at least one further component. The further component(s) may be one or more of the components identified in a method set forth herein or in the Examples section below. Some non-limiting examples of components that can be combined into a kit of the present disclosure are set forth below.

In a particular implementation, a kit can include at least one modified nucleotide or nucleoside labeled with any of the dyes set forth herein together with modified or unmodified nucleotides or nucleosides. For example, modified nucleotides labeled with dyes according to the disclosure may be supplied in combination with unlabeled or native nucleotides, and/or with fluorescently labeled nucleotides or any combination thereof. Accordingly, the kits may comprise modified nucleotides labeled with dyes according to the disclosure and modified nucleotides labeled with other, for example, prior art dye compounds. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, modified nucleotides labeled with a dye compound, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample. When two modified nucleotides labeled with fluorescent dye compounds are supplied in kit form, it is a feature of some implementations that the spectrally distinguishable fluorescent dyes can be excited at the same wavelength, such as, for example by the same laser. When four modified nucleotides labeled with fluorescent dye compounds are supplied in kit form, it is a feature of some implementations that two of the spectrally distinguishable fluorescent dyes can both be excited at one wavelength and the other two spectrally distinguishable dyes can both be excited at another wavelength. Particular excitation wavelengths can be 488 nm and 532 nm.

In one implementation, a kit includes a modified nucleotide labeled with a compound of the present disclosure and a second modified nucleotide labeled with a second dye wherein the dyes have a difference in absorbance maximum of at least 10 nm, particularly 20 nm to 50 nm. More particularly, the two dye compounds have Stokes shifts of between 15-40 nm where “Stokes shift” is the distance between the peak absorption and peak emission wavelengths.

In a further implementation, a kit can further include two other modified nucleotides labeled with fluorescent dyes wherein the dyes are excited by the same laser at 532 nm. The dyes can have a difference in absorbance maximum of at least 10 nm, particularly 20 nm to 50 nm. More particularly the two dye compounds can have Stokes shifts of between 20-40 nm. Particular dyes which are spectrally distinguishable from dyes of the present disclosure and which meet the above criteria are polymethine analogues as described in U.S. Pat. No. 5,268,486 (for example Cy3) or PCT Pub. No. WO 2002/026891 (Alexa 532; Molecular Probes A20106) or unsymmetrical polymethines as disclosed in U.S. Pat. No. 6,924,372, the disclosures of each is incorporated herein by reference in its entirety. Alternative dyes include rhodamine analogues, for example tetramethyl rhodamine and analogues thereof.

In an alternative implementation, the kits of the disclosure may contain nucleotides where the same base is labeled with two different compounds. A first nucleotide may be labeled with a compound of the disclosure. A second nucleotide may be labeled with a spectrally distinct compound, for example a ‘green’ dye absorbing at less than 600 nm. A third nucleotide may be labeled as a mixture of the compound of the disclosure and the spectrally distinct compound, and the fourth nucleotide may be ‘dark’ and contain no label. In simple terms, therefore, the nucleotides 1-4 may be labeled ‘blue’, ‘green’, ‘blue/green’, and dark. To simplify the instrumentation further, four nucleotides can be labeled with two dyes excited with a single laser, and thus the labeling of nucleotides 1-4 may be ‘blue 1’, ‘blue 2’, ‘blue 1/blue 2’, and dark.

Nucleotides may contain two dyes of the present disclosure. A kit may contain two or more nucleotides labeled with dyes of the disclosure. Kits may contain a further nucleotide where the nucleotide is labeled with a dye that absorbs in the region of 520 nm to 560 nm. Kits may further contain an unlabeled nucleotide.

Although kits are exemplified herein in regard to configurations having different nucleotides that are labeled with different dye compounds, it will be understood that kits can include 2, 3, 4 or more different nucleotides that have the same dye compound.

In particular implementations, a kit may include a polymerase enzyme capable of catalyzing incorporation of the modified nucleotides into a polynucleotide. Other components to be included in such kits may include buffers and the like. The modified nucleotides labeled with dyes according to the disclosure, and other any nucleotide components including mixtures of different nucleotides, may be provided in the kit in a concentrated form to be diluted prior to use. In such implementations a suitable dilution buffer may also be included. Again, one or more of the components identified in a method set forth herein can be included in a kit of the present disclosure.

### Methods of Sequencing

Modified nucleotides (or nucleosides) comprising a dye compound according to the present disclosure may be used in any method of analysis such as method that include detection of a fluorescent label attached to a nucleotide or nucleoside, whether on its own or incorporated into or associated with a larger molecular structure or conjugate. In this context the term “incorporated into a polynucleotide” can mean that the 5′ phosphate is joined in phosphodiester linkage to the 3′ hydroxyl group of a second (modified or unmodified) nucleotide, which may itself form part of a longer polynucleotide chain. The 3′ end of a modified nucleotide set forth herein may or may not be joined in phosphodiester linkage to the 5′ phosphate of a further (modified or unmodified) nucleotide. Thus, in one non-limiting implementation, the disclosure provides a method of detecting a modified nucleotide incorporated into a polynucleotide which comprises: (a) incorporating at least one modified nucleotide of the disclosure into a polynucleotide and (b) detecting the modified nucleotide(s) incorporated into the polynucleotide by detecting the fluorescent signal from the dye compound attached to said modified nucleotide(s).

This method can include: a synthetic step (a) in which one or more modified nucleotides according to the disclosure are incorporated into a polynucleotide and a detection step (b) in which one or more modified nucleotide(s) incorporated into the polynucleotide are detected by detecting or quantitatively measuring their fluorescence.

Some implementations of the present application are directed to methods of sequencing including: (a) incorporating at least one labeled nucleotide as described herein into a polynucleotide; and (b) detecting the labeled nucleotide(s) incorporated into the polynucleotide by detecting the fluorescent signal from the fluorescent dye attached to said modified nucleotide(s).

In one implementation, at least one modified nucleotide is incorporated into a polynucleotide in the synthetic step by the action of a polymerase enzyme. However, other methods of joining modified nucleotides to polynucleotides, such as, for example, chemical oligonucleotide synthesis or ligation of labeled oligonucleotides to unlabeled oligonucleotides, can be used. Therefore, the term “incorporating,” when used in reference to a nucleotide and polynucleotide, can encompass polynucleotide synthesis by chemical methods as well as enzymatic methods.

In a specific implementation, a synthetic step is carried out and may optionally comprise incubating a template polynucleotide strand with a reaction mixture comprising fluorescently labeled modified nucleotides of the disclosure. A polymerase can also be provided under conditions which permit formation of a phosphodiester linkage between a free 3′ hydroxyl group on a polynucleotide strand annealed to the template polynucleotide strand and a 5′ phosphate group on the modified nucleotide. Thus, a synthetic step can include formation of a polynucleotide strand as directed by complementary base-pairing of nucleotides to a template strand.

In all implementations of the methods, the detection step may be carried out while the polynucleotide strand into which the labeled nucleotides are incorporated is annealed to a template strand, or after a denaturation step in which the two strands are separated. Further steps, for example chemical or enzymatic reaction steps or purification steps, may be included between the synthetic step and the detection step. In particular, the target strand incorporating the labeled nucleotide(s) may be isolated or purified and then processed further or used in a subsequent analysis. By way of example, target polynucleotides labeled with modified nucleotide(s) as described herein in a synthetic step may be subsequently used as labeled probes or primers. In other implementations, the product of the synthetic step set forth herein may be subject to further reaction steps and, if desired, the product of these subsequent steps purified or isolated.

Suitable conditions for the synthetic step will be well known to those familiar with standard molecular biology techniques. In one implementation, a synthetic step may be analogous to a standard primer extension reaction using nucleotide precursors, including modified nucleotides as described herein, to form an extended target strand complementary to the template strand in the presence of a suitable polymerase enzyme. In other implementations, the synthetic step may itself form part of an amplification reaction producing a labeled double stranded amplification product comprised of annealed complementary strands derived from copying of the target and template polynucleotide strands. Other synthetic steps include nick translation, strand displacement polymerization, random primed DNA labeling, etc. A particularly useful polymerase enzyme for a synthetic step is one that is capable of catalyzing the incorporation of modified nucleotides as set forth herein. A variety of naturally occurring or modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the modified nucleotides according to the disclosure include those described in PCT. Pub. No. WO 2005/024010 or WO 2006/120433, the disclosures of each is incorporated herein by reference in its entirety. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting implementations, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide. Any of a variety of other applications benefitting the use of polynucleotides labeled with the modified nucleotides comprising fluorescent dyes can use modified nucleotides or nucleosides with dyes set forth herein.

In a particular implementation the disclosure provides use of modified nucleotides comprising dye compounds according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template nucleic acid to be sequenced. The identity of the base present in one or more of the added nucleotide(s) can be determined in a detection or “imaging” step as described herein. The identity of the added base may be determined after each nucleotide incorporation step. The sequence of the template may then be inferred using conventional Watson-Crick base-pairing rules. The use of the modified nucleotides labeled with dyes set forth herein for determination of the identity of a single base may be useful, for example, in the scoring of single nucleotide polymorphisms, and such single base extension reactions are within the scope of this disclosure.

In an implementation of the present disclosure, the sequence of a template polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular implementations, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template polynucleotide, and the blocking group prevents further incorporation of nucleotides. Any unincorporated nucleotides can be washed away and the fluorescent signal from each incorporated nucleotide can be “read” optically by suitable means, such as a charge-coupled device using laser excitation and suitable emission filters. The 3′-blocking group and fluorescent dye compounds can then be removed (deprotected) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509, the disclosure of which is incorporated herein by reference in its entirety, discloses a method to sequence polynucleotides immobilized on a solid support.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ hydroxyl group that serves as a primer or initiation point for the addition of further nucleotides in the sequencing reaction. The region of the template to be sequenced will overhang this free 3′ hydroxyl group on the complementary strand. The overhanging region of the template to be sequenced may be single stranded but can be double-stranded, provided that a “nick is present” on the strand complementary to the template strand to be sequenced to provide a free 3′ OH group for initiation of the sequencing reaction. In such implementations, sequencing may proceed by strand displacement. In certain implementations, a primer bearing the free 3′ hydroxyl group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Pub. No. WO 2001/057248 and WO 2005/047301, the disclosures of each is incorporated herein by reference in its entirety. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxyl group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain implementations, the nucleic acid template to be sequenced may be attached to a solid support via any suitable linkage method known in the art, for example via covalent attachment. In certain implementations template polynucleotides may be attached directly to a solid support (e.g., a silica-based support). However, in other implementations of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to silica-based supports are those for example disclosed in PCT Pub. No. WO 2000/006770, the disclosure of which is incorporated herein by reference in its entirety, wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in PCT Pub. No. WO 2005/047301, the disclosure of which is incorporated herein by reference in its entirety. A still further example of solid-supported template polynucleotides is where the template polynucleotides are attached to hydrogel supported upon silica-based or other solid supports, for example, as described in PCT Pub. No. WO 2000/31148, WO 2001/01143, WO 2002/12566, WO 2003/014392, and WO 2000/53812 and U.S. Pat. No. 6,465,178, the disclosures of each is incorporated herein by reference in its entirety.

A particular surface to which template polynucleotides may be immobilized is a polyacrylamide hydrogel. Polyacrylamide hydrogels are described in the references cited above and in PCT Pub. No. WO 2005/065814, the disclosure of which is incorporated herein by reference in its entirety. Specific hydrogels that may be used include those described in PCT. Pub. No. WO 2005/065814 and U.S. Pub. No. 2014/0079923, the disclosures of each is incorporated herein by reference in its entirety. In one implementation, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218, the disclosure of which is incorporated herein by reference in its entirety. Attachment to beads or microparticles can be useful for sequencing applications. Bead libraries can be prepared where each bead contains different DNA sequences. Some libraries and methods for their creation are described in Nature, 437, 376-380 (2005); Science, 309, 5741, 1728-1732 (2005), the disclosures of each is incorporated herein by reference in its entirety. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Modified nucleotides labeled with dye compounds of the present disclosure may be used for sequencing templates on essentially any type of array, including but not limited to those formed by immobilization of nucleic acid molecules on a solid support.

However, the modified nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, PCT Pub. No. WO 1998/44151 and WO 2000/18957, the disclosures of each is incorporated herein by reference in its entirety, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using the modified nucleotides labeled with dye compounds of the disclosure.

The modified nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some implementations. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to PCT Pub. No. WO 2000/06770 and WO 2001/57248, the disclosures of each is incorporated herein by reference in its entirety. Although one use of the modified nucleotides of the disclosure is in sequencing-by-synthesis reactions, the utility of the modified nucleotides is not limited to such methods. In fact, the nucleotides may be used advantageously in any sequencing methodology which requires detection of fluorescent labels attached to nucleotides incorporated into a polynucleotide.

In particular, the modified nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses modified nucleotides labeled with dye compounds which are dideoxynucleotides lacking hydroxyl groups at both of the 3′ and 2′ positions, such modified dideoxynucleotides being suitable for use in Sanger type sequencing methods and the like.

Modified nucleotides labeled with dye compounds of the present disclosure incorporating 3′ blocking groups, it will be recognized, may also be of utility in Sanger methods and related protocols since the same effect achieved by using modified dideoxy nucleotides may be achieved by using modified nucleotides having 3′-OH blocking groups: both prevent incorporation of subsequent nucleotides. Where nucleotides according to the present disclosure, and having a 3′ blocking group are to be used in Sanger-type sequencing methods it will be appreciated that the dye compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

### EXAMPLES

Additional implementations are disclosed in further detail in the following examples, which are not in any way intended to limit the scope of the claims.

### Example 1: Compound I-1: 7-(3-Carboxyazetidinyl-1)-3-(5-chloro-benzoxazol-2-yl)coumarin

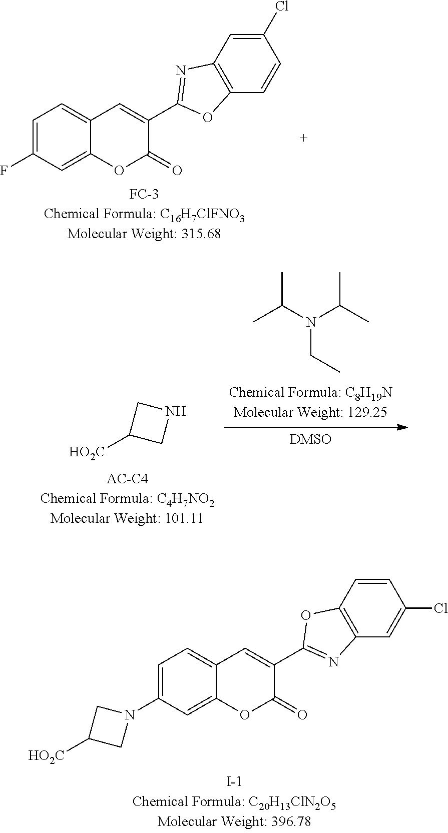

3-(5-Chloro-benzoxazol-2-yl)-7-fluoro-coumarin (0.32 g, 1 mmol) and 3-carboxyazetidine (0.2 g, 2 mmol) were added to anhydrous dimethyl sulfoxide (DMSO, 5 mL) in round bottomed flask. The mixture was stirred for a few minutes at room temperature and then DIPEA (0.52 g, 4 mmol) was added. After stirring for 7 h at 120° C., and standing at room temperature for 1 h, the mixture was diluted with water (15 mL) and stirred overnight. The resulting precipitate was collected by suction filtration. Yield 0.25 g (63%). Purity, structure and composition of the product were confirmed by HPLC, NMR and LCMS. MS (DUIS): MW Calculated 396.05. Found m/z: (+) 397 (M+1)⁺; (−) 395 (M−1)⁻.

### Example 2. Compound I-2: 7-(3-Carboxyazetidin-1-yl)-3-(benzoxazol-2-yl)coumarin

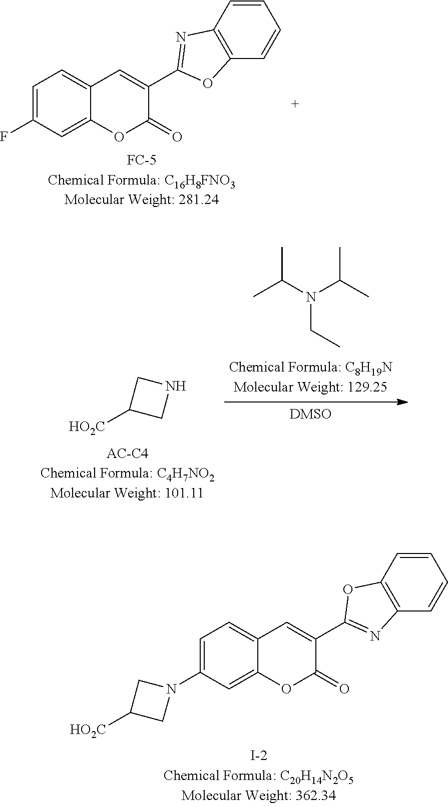

3-(Benzoxazol-2-yl)-7-fluoro-coumarin (0.56 g, 2 mmol) and 3-carboxyazetidine (0.3 g, 3 mmol) is added to anhydrous dimethyl sulfoxide (DMSO, 5 mL) in round bottomed flask. The mixture was stirred for a few minutes at room temperature and then DIPEA (0.52 g, 4 mmol) was added. After stirring for 9 h at 125° C. and standing at room temperature for 1 h, the reaction mixture was diluted with water (10 mL) and stirred overnight. The resulting precipitate was collected by suction filtration. Yield 0.41 g (56%). Purity, structure and composition of the product were confirmed by HPLC, NMR and LCMS. MS (DUIS): MW Calculated 362.09. Found m/z: (+) 363 (M+1)⁺.

### Example 3. Compound I-3: 7-(3-Carboxyazetidin-1-yl)-3-(benzimidazol-2-yl)coumarin

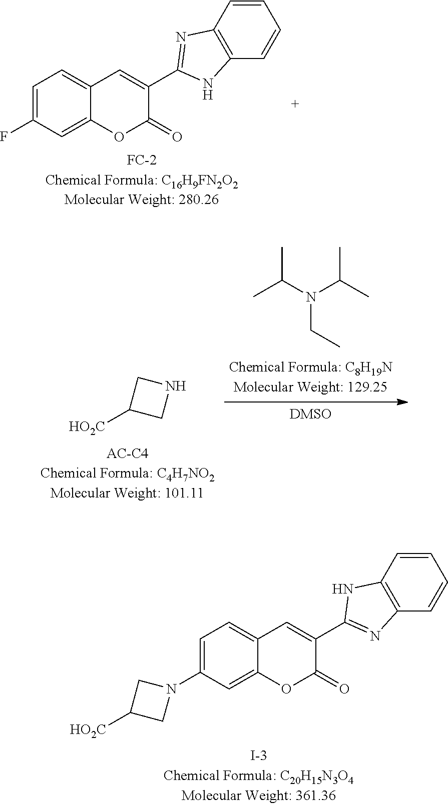

3-(Benzimidazol-2-yl)-7-fluoro-coumarin (FC-2, 0.56 g, 2 mmol, 1 eq.) and 3-carboxyazetidine (AC-C4, 0.3 g, 3 mmol, 1.5 eq) were added to anhydrous dimethyl sulfoxide (DMSO, 5 mL) in round bottomed flask. The mixture was stirred for a few minutes at room temperature and then DIPEA (0.52 g, 4 mmol) was added. The mixture is stirred for 9 h at 120° C. Additional portions of 3-carboxyazetidine (0.3 g, 3 mmol) and DIPEA (0.26 g, 2 mmol) were added. After stirring at 120° C. for another 3 h, and standing at room temperature for 1 h, the reaction mixture was diluted with water (10 mL) and stirred overnight. The resulting precipitate was collected by suction filtration. Yield 0.26 g (36%). Purity, structure and composition of the product were confirmed by HPLC, NMR and LCMS. MS (DUIS): MW Calculated 361.11. Found m/z: (+) 362 (M+1)⁺; (−) 360 (M−1)⁻.

### Example 4. Compound I-4: 7-(3-Carboxyazetidin-1-yl)-3-(benzothiazol-2-yl)coumarin

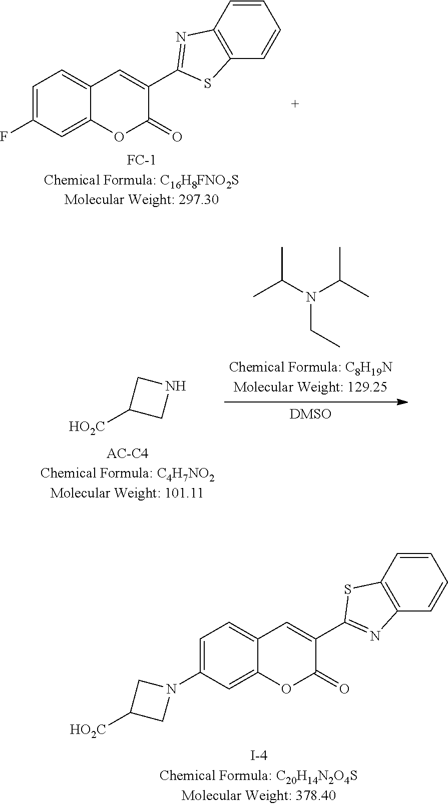

3-(Benzothiazol-2-yl)-7-fluoro-coumarin (0.30 g, 1 mmol) and 3-carboxyazetidine (0.2 g, 2 mmol) were added to anhydrous dimethyl sulfoxide (DMSO, 5 mL) in round bottomed flask. The mixture was stirred for a few minutes at room temperature and then DIPEA (0.52 g, 4 mmol) was added. After stirring for 8 h at 120° C. and standing at room temperature for 1 h, the reaction mixture was diluted with water (10 mL) and was stirred overnight. The resulting precipitate is collected by suction filtration. Yield 0.28 g (75%). Purity, structure and composition of the product were confirmed by HPLC, NMR and LCMS. MS (DUIS): MW Calculated 378.07. Found m/z: (+) 379 (M+1)⁺; (−) 377 (M−1)⁻.

### Example 5. Compound I-5: 7-(3-Carboxypyrrolidin-yl-1)-3-(benzothiazol-2-yl)coumarin

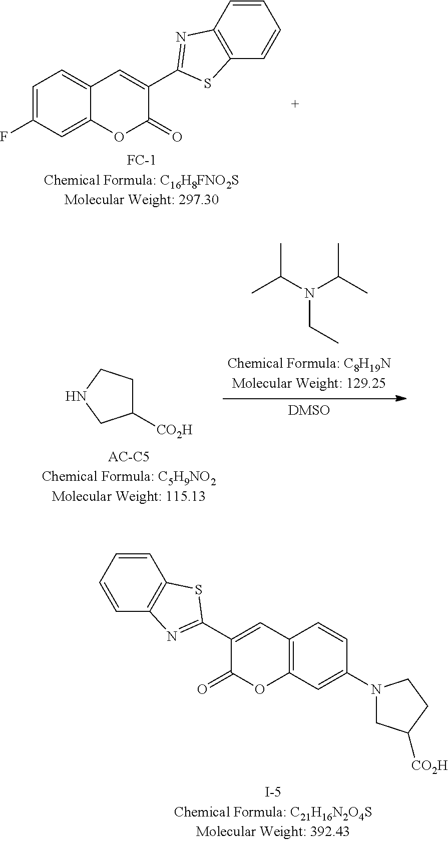

3-(Benzothiazol-2-yl)-7-fluoro-coumarin (0.30 g, 1 mmol) and 3-carboxypyrrolidine (0.23 g, 2 mmol) were added to anhydrous dimethyl sulfoxide (DMSO, 5 mL) in round bottomed flask. The mixture was stirred for a few minutes at room temperature and then DIPEA (0.52 g, 4 mmol) was added. After stirring for 6 h at 120° C. and standing at room temperature for 1 h, the reaction mixture was diluted with water (20 mL) and was stirred overnight. The resulting precipitate was collected by suction filtration. Yield 0.31 g (80%). Purity, structure and composition of the product were confirmed by HPLC, NMR and LCMS. MS (DUIS): MW Calculated 392.08. Found m/z: (+) 393 (M+1)⁺; (−) 391 (M−1)⁻.

### Example 6. Compound I-6: 7-(4-Carboxypiperidin-1-yl)-3-(benzothiazol-2-yl)coumarin

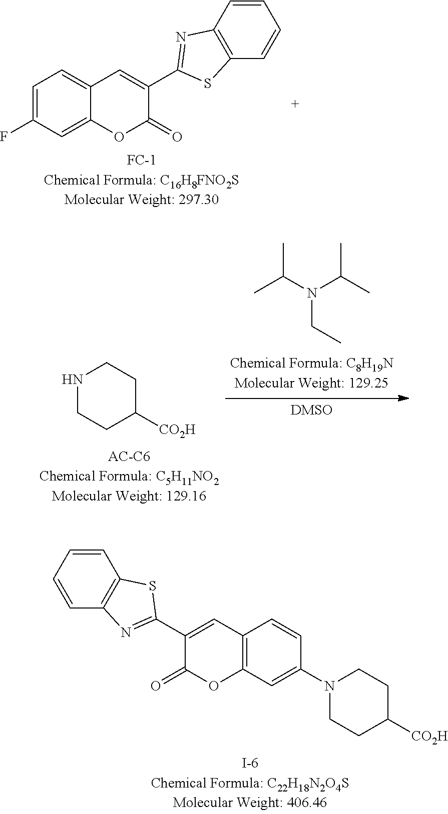

3-(Benzothiazol-2-yl)-7-fluoro-coumarin (0.30 g, 1 mmol) and isonipecotic acid (0.26 g, 2 mmol) were added to anhydrous dimethyl sulfoxide (DMSO, 5 mL) in round bottomed flask. The mixture was stirred for a few minutes at room temperature and then DIPEA (0.52 g, 4 mmol) was added. After stirring for 6 h at 120° C. and standing at room temperature for 1 h, the reaction mixture was diluted with water (20 mL) and was stirred overnight. The resulting precipitate was collected by suction filtration. Yield 0.34 g (83%). Purity, structure and composition of the product were confirmed by HPLC, NMR and LCMS. MS (DUIS): MW Calculated 406.10 Found m/z: (+) 407 (M+1)⁺; (−) 405 (M−1)⁻.

### Example 7. Compound I-7: 7-(3-Carboxyazetidin-1-yl)-3-(6-sulfo-benzothiazol-2-yl)coumarin

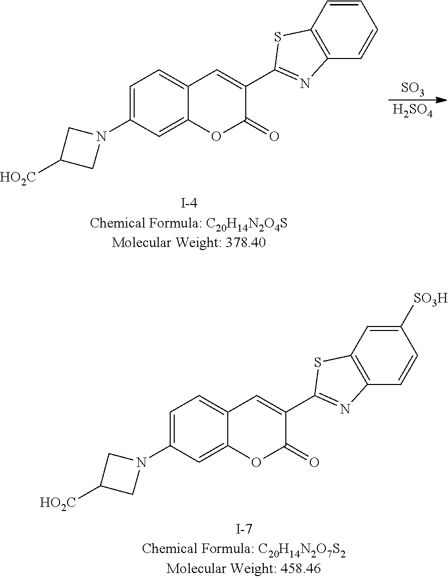

7-(3-Carboxyazetidin-1-yl)-3-(benzothiazol-2-yl)coumarin (0.38 g, 1 mmol) was added at about −5° C. to 20% fuming sulfuric acid (0.5 mL). The mixture was stirred with cooling for a few hours and then at room temperature for 3 h. After stirring for 1 h at 80° C. and standing at room temperature for 1 h, the reaction mixture was diluted with anhydrous diethyl ether (10 mL) and was stirred overnight. The resulting precipitate is collected by suction filtration. Product was purified by HPLC. Yield 0.1 g (22%). Purity, structure and composition of the product were confirmed by HPLC, NMR and LCMS. MS (DUIS): MW Calculated 458.02. Found m/z: (+) 459 (M+1)⁺.

### Example 8. Compound I-8: 7-(3-Carboxyazetidin-1-yl)-3-(6-sulfamido-benzoxazol-2-yl)coumarin

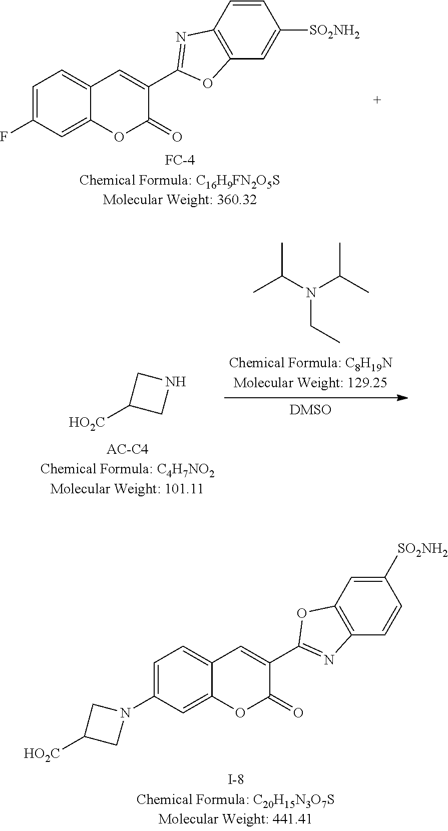

3-(6-Sulfamido-benzoxazol-2-yl)-7-fluoro-coumarin (0.36 g, 1 mmol) and 3-carboxyazetidine (0.3 g, 3 mmol) is added to anhydrous dimethyl sulfoxide (DMSO, 5 mL) in round bottomed flask. The mixture was stirred for a few minutes at room temperature and then DIPEA (0.52 g, 4 mmol) was added. After stirring for 9 h at 125° C. and standing at room temperature for 1 h, the reaction mixture was diluted with water (10 mL) and stirred overnight. The resulting precipitate was collected by suction filtration. Yield 0.26 g (60%). Purity, structure and composition of the product were confirmed by HPLC, NMR and LCMS. MS (DUIS): MW Calculated 441.06. Found m/z: (+) 442 (M+1)⁺.

### Example 9. Comparison of Fluorescence Intensities

Fluorescence intensities of some dye solutions (at maximum excitation wavelength 450 nm) were compared with a standard dye for the same spectral region. The results are shown in Table 1 and demonstrate significant advantages of the dyes for fluorescence based analytical applications.

TABLE 1
Spectral properties of the fluorescent dyes disclosed herein in the examples.

| | | Spectral properties | | |
|---|---|---|---|---|
| | | in EtOH-water 1:1 | | |
| | | | | Relative |
| | | Abs. max | Fluorescence | Fluorescence |
| Number | Structure | (nm) | max (nm) | Intensity (%) |
| I-1 | 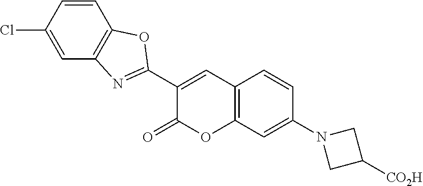 [SMILES: O=C(O)C1CN(c2ccc3cc(-c4nc5cc(Cl)ccc5o4)c(=O)oc3c2)C1] | 451 | 499 | 90 |
| I-2 | 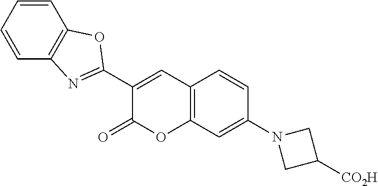 [SMILES: O=C(O)C1CN(c2ccc3cc(-c4nc5ccccc5o4)c(=O)oc3c2)C1] | 446 | 96 | 70 |
| I-3 | 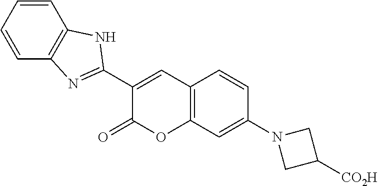 [SMILES: O=C(O)C1CN(c2ccc3cc(-c4nc5ccccc5[nH]4)c(=O)oc3c2)C1] | 443 | 496 | 75 |
| I-4 | 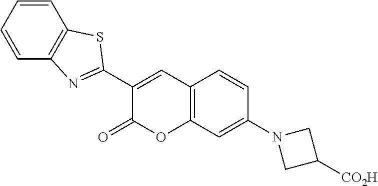 [SMILES: O=C(O)C1CN(c2ccc3cc(-c4nc5ccccc5s4)c(=O)oc3c2)C1] | 449 | 497 | 94 |
| I-5 | 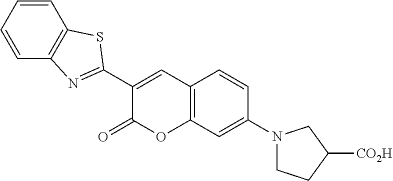 [SMILES: O=C(O)C1CCN(c2ccc3cc(-c4nc5ccccc5s4)c(=O)oc3c2)C1] | 473 | 512 | 138 |
| I-6 | 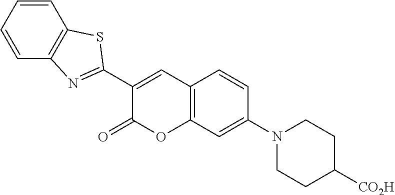 [SMILES: O=C(O)C1CCN(c2ccc3cc(-c4nc5ccccc5s4)c(=O)oc3c2)CC1] | 463 | 514 | 98 |

### Example 10. General Procedure for the Synthesis of Fully Functional Nucleotide Conjugates

Coumarin fluorescent dyes disclosed herein were coupled with appropriate amino-substituted adenine (A) and cytosine (C) nucleotide derivatives A-LN3-NH₂ or C-LN3-NH₂:

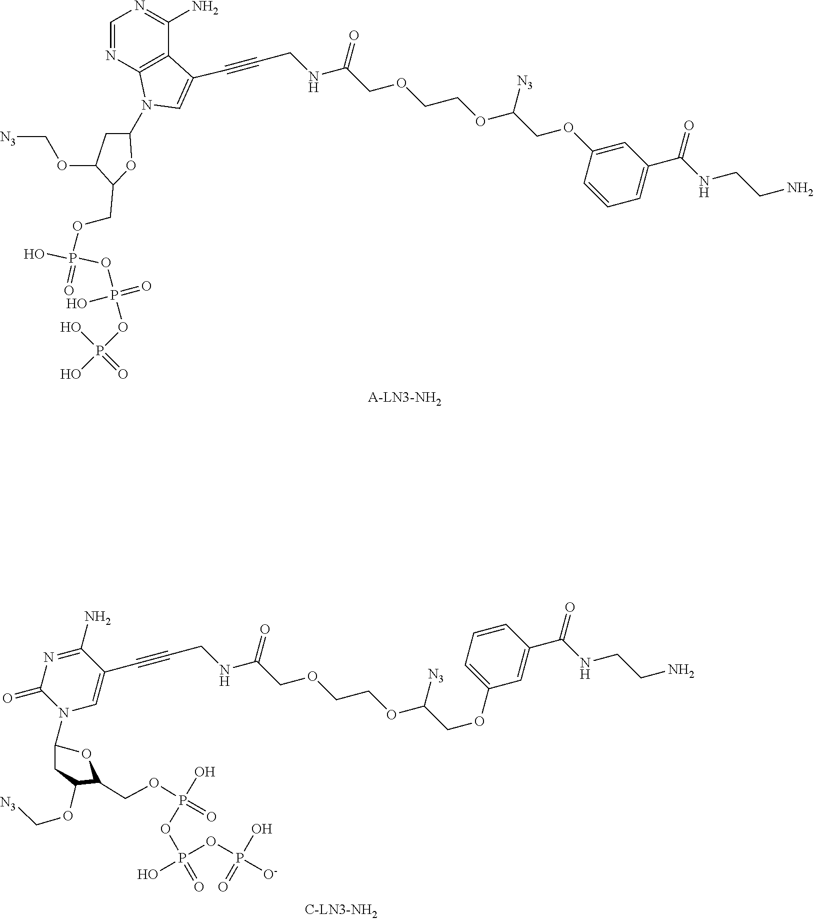

After activation of carboxylic group of a dye with appropriate reagents according to the following adenine scheme:

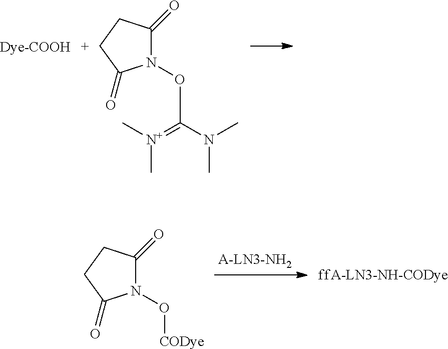

The general product for the adenine coupling is as shown below:

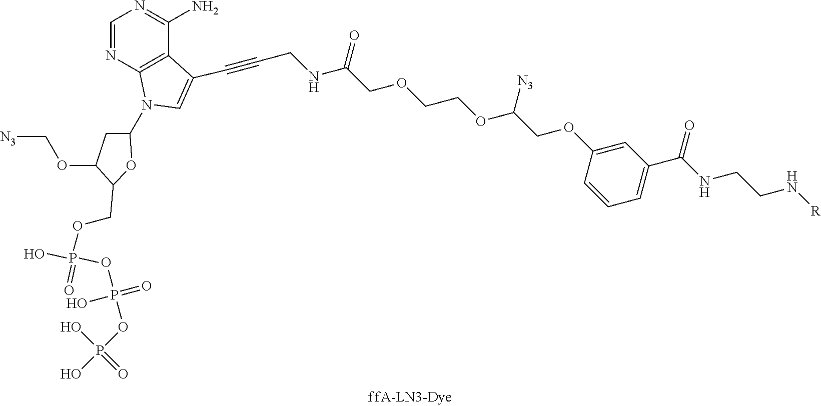

ffA-LN3-Dye refers to a fully functionalized A nucleotide with an LN3 linker and labeled with a coumarin dye disclosed herein. The R group in each of the structures refers to the coumarin dye moiety after conjugation.

The dye (10 μmol) is dried by placing into a 5 mL round-bottomed flask and is dissolved in anhydrous dimethylformamide (DMF, 1 mL) then the solvent is distilled off in vacuo. This procedure is repeated twice. The dried dye is dissolved in anhydrous N,N-dimethylacetamide (DMA, 0.2 mL) at room temperature. N,N,N′,N′-Tetramethyl-O—(N-succinimidyl)uronium tetrafluoroborate (TSTU, 1.5 eq., 15 μmol, 4.5 mg) is added to the dye solution, then DIPEA (3 eq., 30 μmol, 3.8 mg, 5.2 μL) is added via micropipette to this solution. The reaction flask is sealed under nitrogen gas. The reaction progress is monitored by TLC (eluent Acetonitrile-Water 1:9) and HPLC. Meanwhile, a solution of the appropriate amino-substituted nucleotide derivative (A-LN3-NH₂, 20 mM, 1.5 eq, 15 μmol, 0.75 mL) is concentrated in vacuo then re-dissolved in water (20 μL). A solution of the activated dye in DMA is transferred to the flask containing the solution of N-LN3-NH₂. More DIPEA (3 eq, 30 μmol, 3.8 mg, 5.2 μL) is added along with triethylamine (1 μL). Progress of coupling is monitored hourly by TLC, HPLC, and LCMS. When the reaction is complete, triethylamine bicarbonate buffer (TEAB, 0.05 M˜ 3 mL) is added to the reaction mixture via pipette. Initial purification of the fully functionalized nucleotide is carried out by running the quenched reaction mixture through a DEAE-Sephadex® column to remove most of remaining unreacted dye. For example, Sephadex is poured into an empty 25 g Biotage cartridge, solvent system TEAB/MeCN. The solution from the Sephadex column is concentrated in vacuo. The remaining material is redissolved in the minimum volume of water and acetonitrile, before filtering through a 20 m Nylon filter. The filtered solution is purified by preparative-HPLC. The composition of prepared compounds is confirmed by LCMS.

The general product for the cytosine coupling is as shown below, following similar procedure described above.

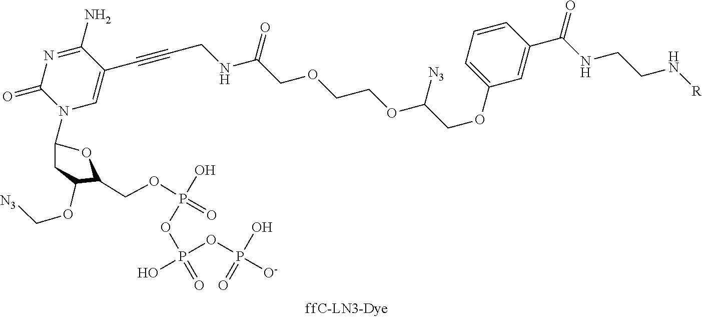

ffC-LN3-Dye refers to a fully functionalized C nucleotide with an LN3 linker and labeled with a coumarin dye disclosed herein. The R group in each of the structures refers to the coumarin dye moiety after conjugation.

### Example 11. Preparation of Amide Derivatives of the Compounds of Formula (I)

Some additional implementations described herein are related to amide derivatives of compounds of Formula (I) and methods of preparing the same, the methods include converting a compound of Formula (Ia) to a compound of Formula (Ia′) through carboxylic acid activation:

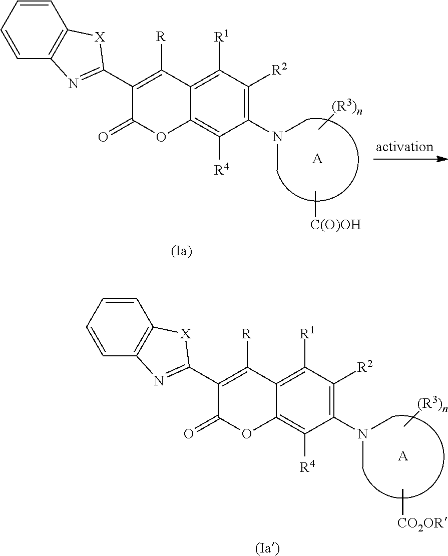

and reacting the compound of Formula (Ia′) with a primary or secondary amine of Formula (Am) to arrive at the amide derivative of Formula (Ib):

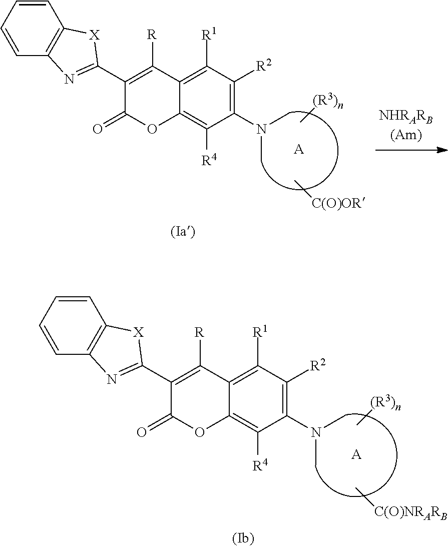

where the variables X, R, R¹, R², R³, R⁴, and n are defined herein; R′ is the residual moiety of a carboxyl activating agent (such as N-hydroxysuccinimide, nitrophenol, pentafluorophenol, HOBt, BOP, PyBOP, DCC, etc.); each of R_{A} and R_{B} is independently hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, 3-10 membered heterocyclyl, aralkyl, heteroaralkyl, or (heterocyclyl)alkyl.

### General Procedure for the Preparation of Compounds of Formula (Ib)

An appropriate dye of Formula (Ia) (0.001 mol) is dissolved in suitable anhydrous organic solvent (DMF, 1.5 mL). To this solution a carboxyl activating reagent such as TSTU, BOP or PyBOP is added. This reaction mixture is stirred at room temperature for about 20 min and then appropriate amine derivatives is added. The reaction mixture is stirred overnight, filtered and excess of the activation reagent is quenched with 0.1M TEAB solution in water. Solvents is evaporated in vacuum and the residue is re-dissolved in TEAB solution and purified by HPLC.

### Example 12. Two-Channel Sequencing Applications

The efficiency of the A nucleotides labeled with the dyes described herein in sequencing application was demonstrated in the two-channel detection method as described herein. With respect to the two-channel methods described herein, nucleic acids can be sequenced utilizing methods and systems described herein and/or in U.S. Pat. Pub. No. 2013/0079232, the disclosure of which is incorporated herein by reference in its entirety.

In the two-channel detection, a nucleic acid can be sequenced by providing a first nucleotide type that is detected in a first channel, a second nucleotide type that is detected in a second channel, a third nucleotide type that is detected in both—the first and the second channel and a fourth nucleotide type that lacks a label that is not, or minimally, detected in either channel. The scatterplots were generated by RTA2.0.93 analysis of an experiment. The scatterplots illustrated in FIG. 23 through FIG. 25 were at cycle 5 of each of the 26 cycle runs.

FIG. 23 illustrates the scatterplot of a fully functionalized nucleotides (ffN) mixture containing: A-I-4 (0.5 μM), A-NR550S0 (1.5 μM), C—NR440 (2 μM), dark G (2 μM) and T-AF550POPOS0 (2 μM) in incorporation buffer with Pol812. Blue exposure (Chanel 1) 500 ms, Green exposure (Chanel 2) 1000 ms; Scanned in Scanning mix).

FIG. 24 illustrates the scatterplot of a fully functionalized nucleotides (ffN) mixture containing: A-I-S (1 μM), A-NR550S0 (1 μM), C—NR440 (2 μM), dark G (2 μM) and T-AF550POPOS0 (2 μM) in incorporation buffer with Pol812. Blue exposure (Chanel 1) 500 ms, Green exposure (Chanel 2) 1000 ms; Scanned in Scanning mix.

FIG. 25 illustrates the scatterplot of a fully functionalized nucleotides (ffN) mixture containing: A-I-6 (1 μM), A-NR550S0 (1 μM), C—NR440 (2 μM), dark G (2 μM) and T-AF550POPOS0 (2 μM) in incorporation buffer with Pol812. Blue exposure (Chanel 1) 500 ms, Green exposure (Chanel 2) 1000 ms; Scanned in Scanning mix.

In each of FIGS. 23-25, “G” nucleotide is unlabeled and shown as the lower left cloud (“dark G”). The signal from a mixture of “A” nucleotide labeled by the dyes described herein and a green dye (NR550S0) is shown as the upper right cloud in FIGS. 23-25 respectively. The signal from the “T” nucleotide labelled with dye AF550POPOS0 is indicated by the upper left cloud, and signal from “C” nucleotide labelled by dye NR440 is indicated by the lower right cloud. The X-axis shows the signal intensity for one (Blue) channel and the Y-axis shows the signal intensity for the other (Green) channel. The chemical structures of NR440, AF550POPOS0, and NR550S0 are disclosed in PCT Pub. No. WO 2018/060482, WO 2017/051201, and WO 2014/135221 respectively, the disclosures of each is incorporated herein by reference in its entirety.

FIGS. 23-25 each shows that the fully functional A-nucleotide conjugates labelled with the dye described herein provides sufficient signal intensities and great cloud separation.

### Example 13. Compound II-1: 7-Bis(2-Carboxyethyl)amino-3-(5-chloro-benzoxazol-2-yl)coumarin

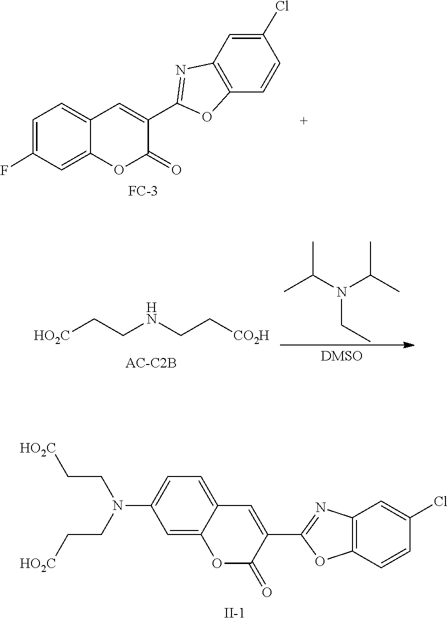

3-(5-Chloro-benzoxazol-2-yl)-7-fluoro-coumarin (0.32 g, 1 mmol) and bisiminopropionic acid (0.32 g, 2 mmol) were added to anhydrous DMSO (5 mL). The resulting mixture was stirred for a few minutes at room temperature and DIPEA (0.52 g, 4 mmol) was added. The resulting mixture was stirred for 6 hours at 130° C. After standing at room temperature for ˜1 h, the pale-yellow reaction mixture was diluted with water (15 mL) and stirred overnight. The resulting precipitate was collected by suction filtration. Yield: 0.40 g (88%). Purity, structure and composition of the product were confirmed by HPLC, NMR and LCMS. MS (DUIS): MW Calculated 456.07. Found m/z: (+) 427 (M+1).

### Example 14. Compound II-2: 7-Diethylamino-3-(5-carboxy-benzoxazol-2-yl)coumarin

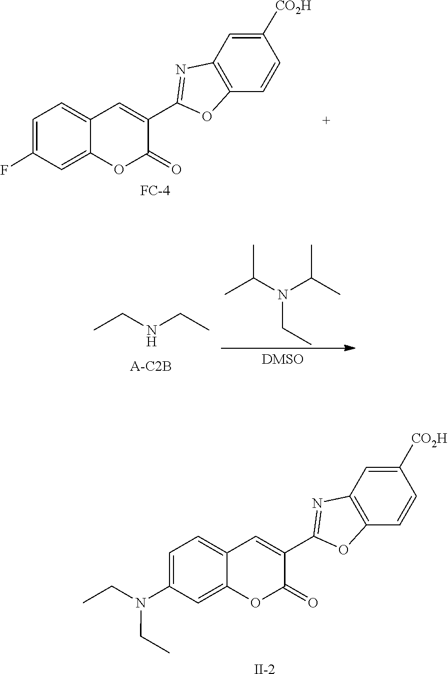

3-(5-Carboxybenzoxazol-2-yl)-7-fluoro-coumarin (0.33 g, 1 mmol) and diethylamine (0.29 g, 4 mmol) were added to anhydrous DMSO (15 mL). The resulting mixture was stirred for a few minutes at room temperature and DIPEA (0.52 g, 4 mmol) was added. The reaction mixture was stirred with a condenser for 12 h at 115° C. Additional portions of diethylamine (0.14 g, 2 mmol) and DIPEA (0.26 g, 2 mmol) were added and stirring at 115° C. was continued for 5 h. Half the volume of solvent was then distilled off under vacuum and the resulting mixture was left to stand at room temperature for 1 h. The resulting mixture was diluted with water (15 mL) and stirred overnight. The resulting precipitate was collected by suction filtration and washed with water. Yield 0.24 g (62%). Purity, structure and composition of the product were confirmed by HPLC, NMR and LCMS. MS (DUIS): MW Calculated 378.12. Found m/z: (+) 379 (M+1)⁺; (−) 377 (M−1)⁻.

Alternative Synthesis

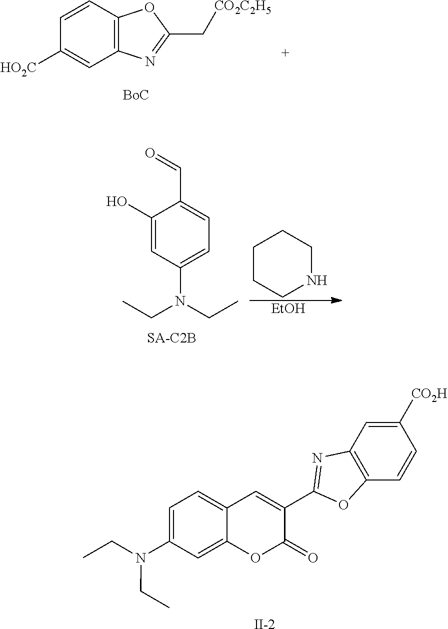

Ethyl (5-carboxybenzoxazol-2-yl)acetate (0.25 g, 1 mmol), diethylaminosalisylic aldehyde (0.19 g, 1 mmol), piperidine (3 drops), and acetic acid (3 drops) were added to anhydrous ethanol (EtOH, 5 mL) in round-bottomed flask. The resulting mixture was stirred for 6 h at room temperature and then at 60-65° C. for 12 h. The resulting precipitate was collected by suction filtration and washed with water. Yield: 0.27 g (72%). Purity, structure and composition of the product were confirmed by HPLC, NMR and LCMS. MS (DUIS): MW Calculated 378.12. Found m/z: (+) 379 (M+1)⁺; (−) 377 (M−1)⁻.

### Example 15. Compound II-3: 7-Diethylamino-3-(5-carboxy-benzimidazol-2-yl)coumarin

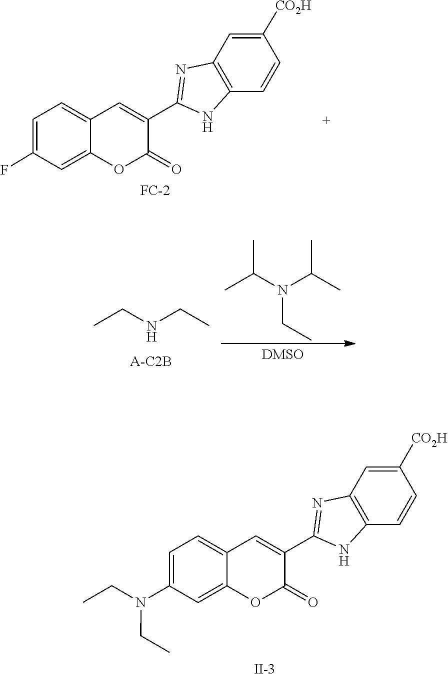

3-(5-Carboxybenzimidazol-2-yl)-7-fluoro-coumarin (0.32 g, 1 mmol) and diethylamine (0.29 g, 4 mmol) were added to anhydrous dimethyl sulfoxide (DMSO, 15 mL) in round bottomed flask. After the addition was complete, the mixture was stirred for a few minutes at room temperature and then DIPEA (0.52 g, 4 mmol) was added. The reaction mixture was stirred with a condenser for 12 h at 115° C. Additional portions of diethylamine (0.14 g, 2 mmol) and DIPEA) 0.26 g, 2 mmol) were added and the mixture was heated at 115° C. for another 8 h. Half the volume of solvent was distilled off under vacuum. After standing at room temperature for 1 h, the mixture was diluted with water (15 mL) and stirred overnight. The resulting precipitate was collected by suction filtration and washed with water. Yield: 0.17 g (44%). Purity, structure and composition of the product were confirmed by HPLC, NMR and LCMS. MS (DUIS): MW Calculated 377.14. Found m/z: (+) 378 (M+1)⁺; (−) 376 (M−1)⁻.

Alternative Synthesis

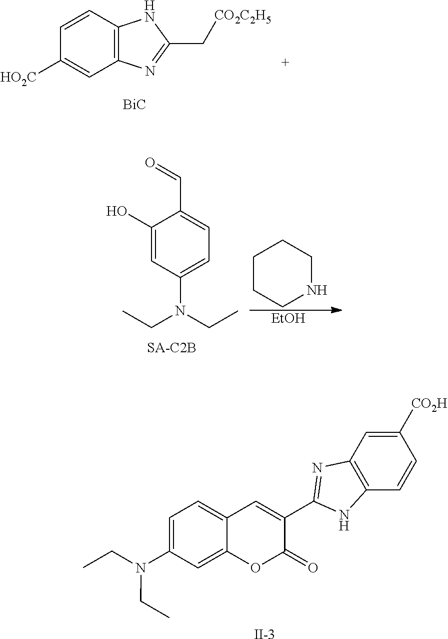

Ethyl(5-carboxybenzimidazol-2-yl)acetate (0.25 g, 1 mmol), diethylaminosalisylic aldehyde (0.19 g, 1 mmol), piperidine (3 drops), and acetic acid (3 drops) were added to anhydrous ethanol (EtOH, 5 mL) in round bottomed flask. The resulting mixture was stirred overnight at 75° C. The resulting precipitate was collected by suction filtration and washed with water. Yield: 0.26 g (70%). Purity, structure and composition of the product were confirmed by HPLC, NMR and LCMS. MS (DUIS): MW Calculated 377.14. Found m/z: (+) 378 (M+1)⁺; (−) 376 (M−1)⁻.

### Example 16. Compound II-4: 7-[N-(3-Carboxypropyl)-N-methyl]amino-3-(benzthiazol-2-yl)coumarin

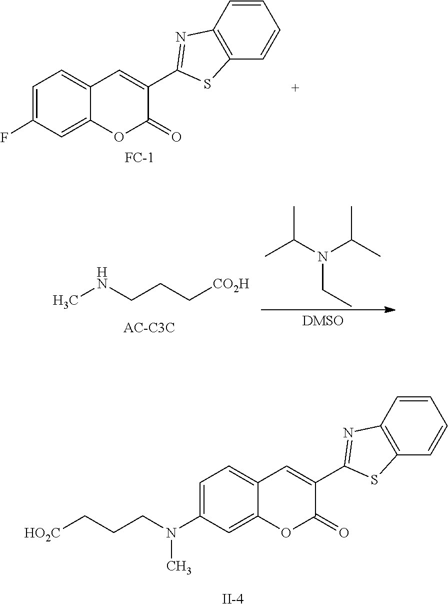

3-(Benzothiazol-2-yl)-7-fluoro-coumarin (0.30 g, 1 mmol) and 4-(methylamino)butanoic acid (0.23 g, 2 mmol) were added to anhydrous DMSO (5 mL) in round bottomed flask. The mixture was stirred for a few minutes at room temperature and then DIPEA (0.52 g, 4 mmol) was added. The reaction mixture was stirred for 8 h at 120° C. and then at room temperature for about 1 h. The pale-yellow mixture was diluted with water (15 mL) and stirred overnight. The resulting precipitate was collected by suction filtration. Yield: 0.19 g (48%). Purity, structure and composition of the product were confirmed by HPLC, NMR and LCMS. MS (DUIS): MW Calculated 456.07. Found m/z: (+) 427 (M+1).

### Example 17. Compound II-5: 7-[N-(3-Carboxypropyl)-N-(3-sulfopropyl)amino]-3-(benzothiazol-2-yl)coumarin (triethylammonium salt)

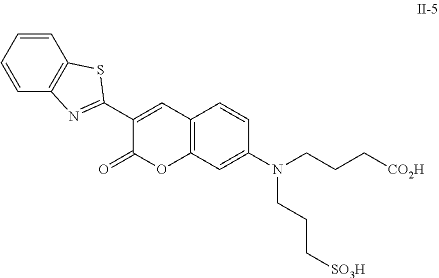 [SMILES: O=C(O)CCCN(CCCS(=O)(=O)O)c1ccc2cc(-c3nc4ccccc4s3)c(=O)oc2c1]

### Step 1: Preparation of 7-{N-[3-(t-Butyloxycarbonyl)propyl]-N-(3-sulfopropyl]}amino-3-(benzothiazol-2-yl)coumarin (Compound II-5tBu)

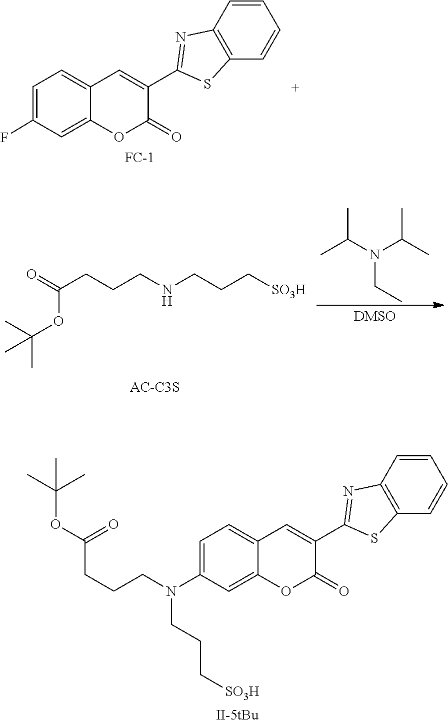

3-(Benzothiazol-2-yl)-7-fluoro-coumarin (0.3 g, 1 mmol) and t-butyl 4-[N-(3-sulfo)propyl]-aminobutanoate (0.56 g, 2 mmol) was added to anhydrous DMSO (3 mL) in round bottomed flask. The mixture was stirred for a few minutes at room temperature and then DIPEA (0.65 g, 5 mmol) was added to this mixture. The reaction mixture was stirred for 3 h at 120° C. Half the volume of the solvent was distilled of under vacuum. The mixture was left standing room temperature for 1 h, and the resulting mixture was diluted with water (10 mL) and the product Compound II-5tBu was isolated as the triethylammonium salt by preparative HPLC with acetonitrile-TEAB mixture as an eluent. Yield 0.5 g (76%). Purity, structure and composition were confirmed by HPLC, NMR and LCMS. MS (DUIS): MW Calculated 558.15. Found m/z: (+) 559 (M+1).

Step 2: Trifluoroacetic acid (3 mL) was added to a mixture of triethylammonio 7-{N-[3-(t-butyloxycarbonyl)propyl]-N-[(3-sulfonatopopyl]}amino-3-(benzothiazol-2-yl)coumarin (0.66 g, 1 mmol) in anhydrous dichloromethane (25 mL), and the mixture was stirred for 24 h at room temperature. The solvents were removed by distillation. The residue was dissolved in an acetonitrile-water mixture (1:1.10 mL) and the product was isolated as Compound II-5 triethylammonium salt by preparative HPLC with acetonitrile-TEAB mixture as an eluent. Yield: 0.6 g (97%). Purity, structure and composition were confirmed by HPLC, NMR and LCMS. MS (DUIS): MW Calculated 502.09. Found m/z: (+) 503 (M+1)⁺; (−), 501 (M−1)⁻.

### Example 18. Compound II-6: 7-[N-(3-Carboxypropyl)-N-(3-sulfopropyl)amino]-3-(5-chloro-benzoxazol-2-yl)coumarin (triethylammonium salt)

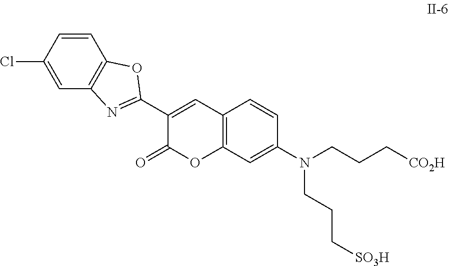 [SMILES: O=C(O)CCCN(CCCS(=O)(=O)O)c1ccc2cc(-c3nc4cc(Cl)ccc4o3)c(=O)oc2c1]

### Step 1. Preparation of 7-{N-[3-(t-Butyloxycarbonyl)propyl]-N-(3-sulfopropyl]}amino-3-[5-chlorobenzoxazol-2-yl)coumarin (Compound II-6tBu)

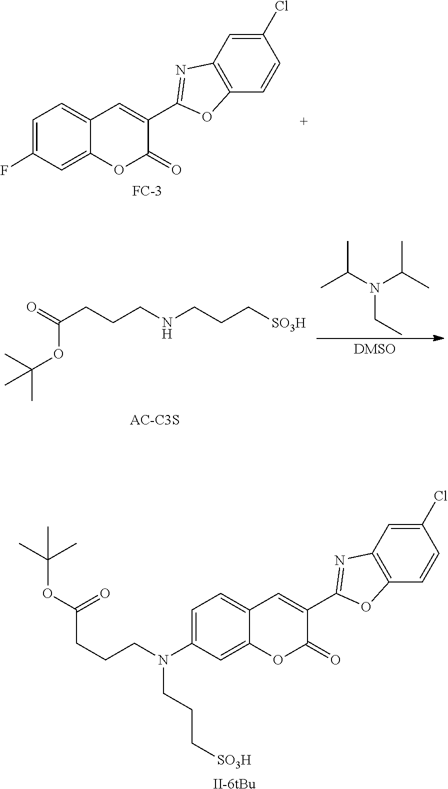

3-5-Chloro-benzoxazol-2-yl)-7-fluoro-coumarin (0.32 g, 1 mmol) an t-butyl 4-[N-(3-sulfo)propyl]-aminobutanoate (0.56 g, 2 mmol) were added to anhydrous DMSO (5 mL) in round bottomed flask. The resulting mixture was stirred for a few minutes at room temperature and then DIPEA (0.65 g, 5 mmol) was added to this mixture. After stirring for 5 hours at 125° C., half the volume of the solvent was distilled off under vacuum. The mixture was left standing at room temperature for 1 h, then was diluted with a water-acetonitrile 1:1 mixture (10 mL), and the product Compound II-6tBu was isolated as the triethylammonium salt by preparative HPLC with acetonitrile-TEAB mixture as an eluent. Yield: 0.38 g (56%). Purity, structure and composition were confirmed by HPLC, NMR and LCMS. MS (DUIS): MW Calculated 576.13. Found m/z: (+) 577 (M+1).

Step 2. A mixture of triethylammonio 7-{N-[3-(t-butyloxycarbonyl)propyl]-N-[(3-sulfonatopopyl]}amino-3-(5-chloro-benzoxazol-2-yl)coumarin (0.68 g, 1 mmol) in anhydrous dichloromethane (25 mL) was treated with trifluoroacetic acid (3 mL) and the resulting mixture was stirred for 24 h at room temperature. The solvents were distilled off, the residue was dissolved in acetonitrile-water 1:1 mixture (10 mL), and the product is isolated as Compound II-6 triethylammonium salt by preparative HPLC with acetonitrile-TEAB mixture as an eluent. Yield: 0.6 g (96%). Purity, structure and composition were confirmed by HPLC, NMR and LCMS. MS (DUIS): MW Calculated 520.07. Found m/z: (+) 521 (M+1)⁺; (−), 519 (M−1)⁻.

### Example 18. Compound II-7: 7-[N-(3-Carboxypropyl)-N-(3-sulfopropyl)amino]-3-(benzoxazol-2-yl)coumarin (isolated as triethylammonium salt)

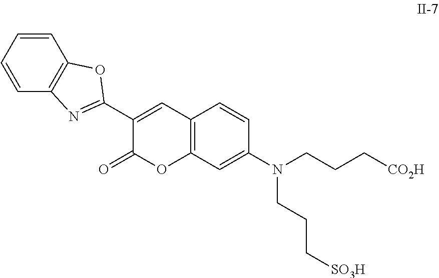 [SMILES: O=C(O)CCCN(CCCS(=O)(=O)O)c1ccc2cc(-c3nc4ccccc4o3)c(=O)oc2c1]

### Step 1. Preparation of 7-{N-[3-(t-Butyloxycarbonyl)propyl]-N-(3-sulfopropyl]}amino-3-(benzoxazol-2-yl)coumarin (Compound II-7tBu)

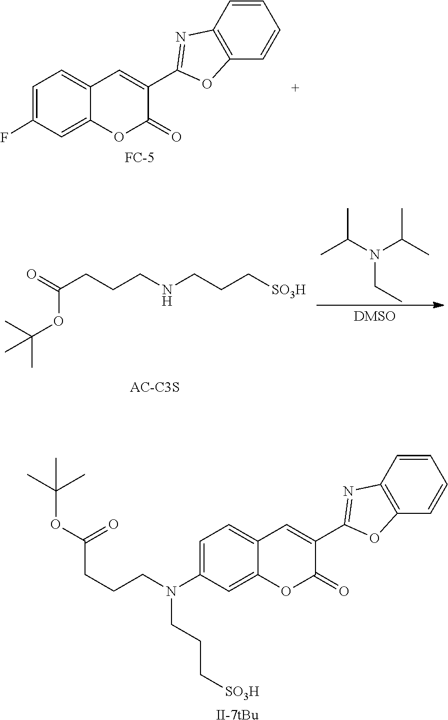

3-(Benzoxazol-2-yl)-7-fluoro-coumarin (0.28 g, 1 mmol) and t-butyl 4-[N-(3-sulfo)propyl]-aminobutanoate (0.56 g, 2 mmol) were added to anhydrous DMSO (5 mL) in round bottomed flask. The resulting mixture was stirred for a few minutes at room temperature and then DIPEA (0.65 g, 5 mmol) was added to this mixture. After stirring for 8 hours at 120° C., half the volume of the solvent was distilled off under vacuum. The mixture was left standing at room temperature for 1 h, then was diluted with a water-acetonitrile 1:1 mixture (10 mL), and the product Compound II-7tBu was isolated by preparative HPLC with acetonitrile-TEAB mixture as an eluent. Yield: 0.15 g (27%). Purity, structure and composition were confirmed by HPLC, NMR and LCMS. MS (DUIS): MW Calculated 542.17. Found m/z: (+) 543 (M+1).

Step 2. A mixture of 7-{N-[3-(t-butyloxycarbonyl)propyl]-N-[(3-sulfonatopopyl]}amino-3-(benzoxazol-2-yl)coumarin (0.27 g, 0.5 mmol) in anhydrous dichloromethane (15 mL) was treated with trifluoroacetic acid (2 mL) and the resulting mixture was stirred for 24 h at room temperature. The solvents were distilled off, the residue was dissolved in acetonitrile-water 1:1 mixture (10 mL), and the product was isolated as triethylammonium salt by preparative HPLC with acetonitrile-TEAB mixture as an eluent. Yield: 87%.

### Example 19. Compound II-8: 7-[N-(3-Carboxypropyl)-N-(3-sulfopropyl)amino]-3-[6-(aminosulfonyl)benzoxazol-2-yl]coumarin

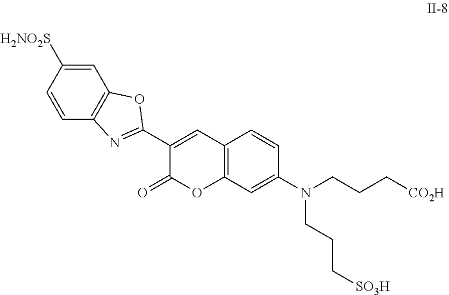 [SMILES: NS(=O)(=O)c1ccc2nc(-c3cc4ccc(N(CCCC(=O)O)CCCS(=O)(=O)O)cc4oc3=O)oc2c1]

### Step 1. Preparation of 7-{N-[3-(t-Butyloxycarbonyl)propyl]-N-(3-sulfopropyl]}amino-3-[6-(aminosulfonyl)benzoxazol-2-yl]coumarin (Compound II-8tBu)

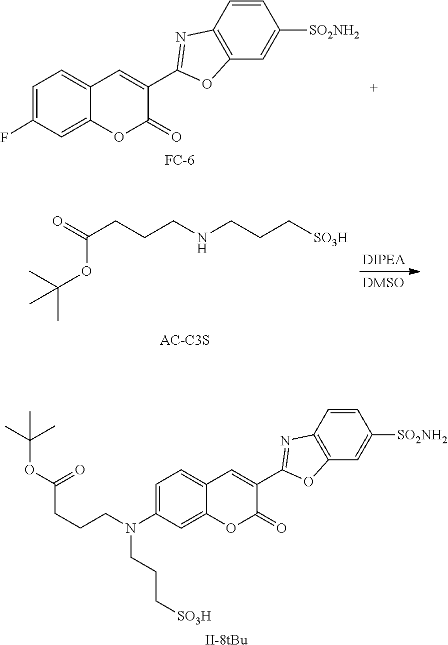

3-[6-(Aminosulfonyl)benzoxazol-2-yl]-7-fluoro-coumarin (0.18 g, 0.5 mmol) and t-butyl 4-[N-(3-sulfo)propyl]-aminobutanoate (0.28 g, 1 mmol) were mixed with anhydrous DMSO (3 mL) in round bottomed flask. The resulting mixture was stirred for a few minutes at room temperature and then DIPEA (0.65 g, 5 mmol) was added. After stirring for 7 hours at 120° C., half the volume of the solvent was distilled off under vacuum. The mixture was left standing at room temperature for one hour, then was diluted with a water-acetonitrile 1:1 mixture (10 mL), and the product Compound II-8tBu was isolated by preparative HPLC with acetonitrile-TEAB mixture as an eluent. After evaporation of solvents yellow precipitate was filtered off. Yield: 0.31 g (50%). Purity, structure and composition of the dye were confirmed by HPLC, NMR and LCMS. MS (DUIS): MW Calculated 621.15. Found m/z: (+) 622 (M+1).

Step 2. To a mixture of 7-{N-[3-(t-butyloxycarbonyl)propyl]-N-[(3-sulfonatopopyl]}amino-3-[6-(aminosulfonyl)benzoxazol-2-yl]coumarin (0.31 g, 0.5 mmol) in anhydrous dichloromethane (15 mL) trifluoroacetic acid (2 mL) was added and the resulting solution was stirred for 24 h at room temperature. The solvents were distilled off, the residue was dissolved in acetonitrile-water 1:1 mixture (10 mL), and the solvents were distilled off again. Compound II-8 was filtered off and washed with acetonitrile. Yield: 0.25 g (87%).

### Example 20. Compound II-9: 7-[N-(3-Carboxypropyl)-N-(3-sulfopropyl)amino]-3-(5-chloro-benzimidazolyl-2-yl)coumarin

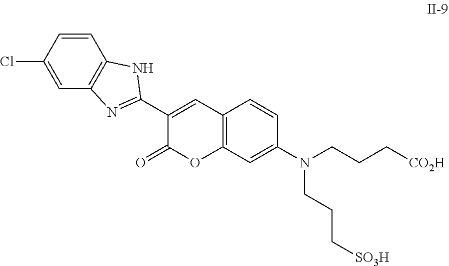 [SMILES: O=C(O)CCCN(CCCS(=O)(=O)O)c1ccc2cc(-c3nc4cc(Cl)ccc4[nH]3)c(=O)oc2c1]

### Step 1. Preparation of 7-{N-[3-(t-Butyloxycarbonyl)propyl]-N-(3-sulfopropyl]}amino-3-[(5-chlorobenzimidazolyl-2-yl)coumarin (Compound II-9tBu)

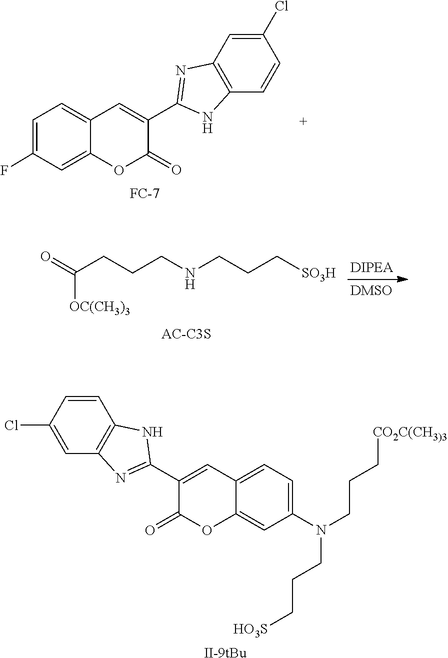

3-(5-Chlorobenzimidazolyl-2-yl)-7-fluoro-coumarin (0.32 g, 1 mmol) and t-butyl 4-(N-3-sulfopropyl)aminobutanoate (0.56 g, 2 mmol) were added to anhydrous DMSO (5 mL) in round bottomed flask. The resulting mixture was stirred for a few minutes at room temperature and then DIPEA (0.65 g, 5 mmol) was added to this mixture. After stirring for 15 hours at 120° C., a half the volume of the solvent was distilled off under vacuum. The mixture was left standing at room temperature for 1 h, then was diluted with a water-acetonitrile 1:1 mixture (10 mL), and the product Compound II-9tBu was isolated as the triethylammonium salt by preparative HPLC with acetonitrile-TEAB mixture as an eluent.

Step 2. Triethylammonio 7-{N-[3-(t-butyloxycarbonyl)propyl]-N-(3-sulfonatopopyl)}amino-3-(5-chlorobenzimidazolyl-2-yl)coumarin from previous step was dissolved in anhydrous dichloromethane (25 mL) and trifluoroacetic acid (5 mL) was added. The resulting mixture was stirred for 24 h at room temperature. The solvents were distilled off, the residue was dissolved in acetonitrile-water 1:1 mixture (10 mL), and the product was isolated by preparative HPLC with acetonitrile-TEAB mixture as an eluent. Yield: 0.2 g (35%). Purity, structure and composition were confirmed by HPLC, NMR and LCMS. MS (DUIS): MW Calculated 519.09. Found m/z: (+) 520 (M+1)⁺; (−), 518 (M−1).

### Example 21. Compound II-10tBu: 7-[N-(3-Carboxypropyl)-N-(3-sulfopropyl)amino]-3-(5-carboxybenzoxazol-2-yl)coumarin

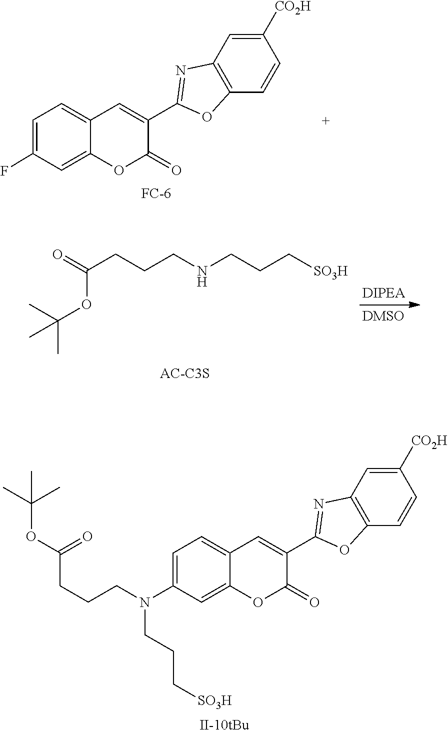

3-(5-Carboxybenzoxazol-2-yl)-7-fluoro-coumarin (0.17 g, 0.5 mmol) and t-butyl 4-(N-3-sulfopropyl)aminobutanoate (0.28 g, 1 mmol) were mixed with anhydrous DMSO (5 mL) in round bottomed flask. The resulting mixture was stirred for a few minutes at room temperature and then DIPEA (0.65 g, 5 mmol) was added. After stirring for 17 hours at 110° C., half the volume of the solvent was distilled off under vacuum. The mixture was left standing at room temperature for one hour, then was diluted with a water-acetonitrile 1:1 mixture (10 mL), and the product Compound II-10tBu was isolated by preparative HPLC with acetonitrile-TEAB mixture as an eluent. After evaporation of solvents yellow precipitate was filtered off. Yield: 0.23 g (80%). Purity, structure and composition of the dye were confirmed by HPLC, NMR and LCMS. MS (DUIS): MW Calculated 586.16. Found m/z: (+) 587 (M+1).

### Example 21. Compound II-11tBu: 7-[N-(3-Carboxypropyl)-N-(3-sulfopropyl)amino]-3-(6-carboxybenzoxazol-2-yl)coumarin

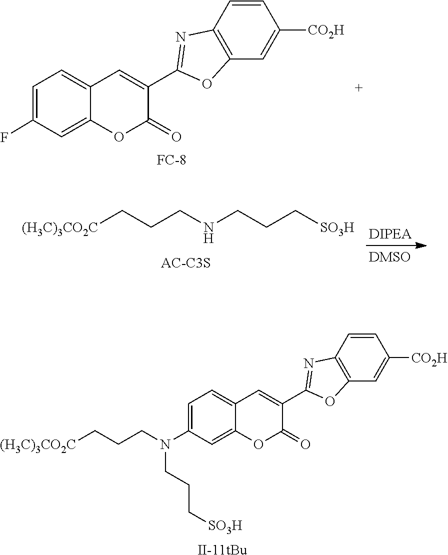

3-(6-Carboxybenzoxazol-2-yl)-7-fluoro-coumarin (0.65 g, 2 mmol) and t-butyl 4-(N-3-sulfopropyl)aminobutanoate (1.13 g, 4 mmol) and anhydrous DMSO (15 mL) was stirred for a few minutes at room temperature and then DIPEA (1.3 g, 10 mmol) was added. After stirring for 15 hours at 120° C., half the volume of the solvent was distilled off under vacuum. The mixture was left stirred at room temperature for one hour, then was diluted with a water-acetonitrile 1:1 mixture (10 mL), and the product Compound II-11tBu was isolated by preparative HPLC with acetonitrile-TEAB mixture as an eluent. After evaporation of solvents yellow precipitate was filtered off. Yield: 0.66 g (56%). Purity, structure and composition of the dye were confirmed by HPLC, NMR and LCMS. MS (DUIS): MW Calculated 586.16. Found m/z: (+) 587 (M+1).

### Example 22. Compound II-12: 7-Diethylamino-3-(5-carboxy-benzothiazol-2-yl)coumarin

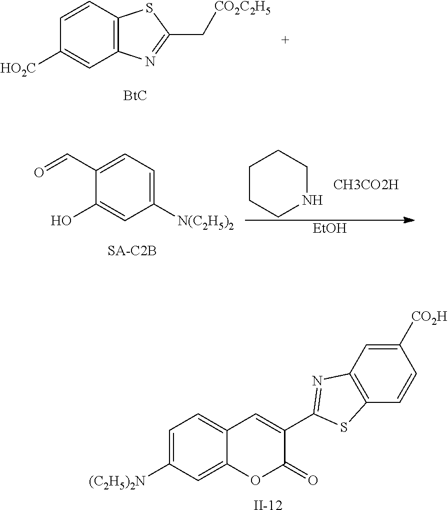

Ethyl (5-carboxybenzthiazol-2-yl)acetate (0.27 g, 1 mmol), diethylamino salicylic aldehyde (0.21 g, 1.1 mmol), piperidine (5 drops), and acetic acid (5 drops) were added to anhydrous ethanol (5 mL) and the resulting mixture was stirred 7 h at 60-65° C. and then left at room temperature overnight. The resulting orange precipitate was collected by suction filtration and washed with water. Yield: 0.28 g (72%).

Alternative Synthesis

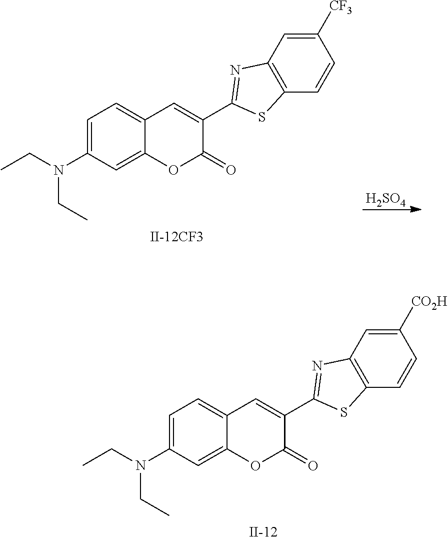

7-Diethylamino-3-(5-Carboxybenzoxazol-2-yl)coumarin (0.84 g, 2 mmol) and concentrated sulfuric acid (5 mL) was stirred for a few minutes at room temperature and then solution was heated for 2 hours at 150° C. The mixture was left stirred at room temperature for one hour, then was diluted with ice-water (50 g) and the reaction mixture was left stirred overnight. Yellow precipitate was filtered off Yield: 0.51 g (65%). Purity, structure and composition of the product were confirmed by HPLC, NMR and LCMS. MS (DUIS): MW Calculated 394.10. Found m/z: (+) 395 (M+1)⁺; (−) 393 (M−1).

### Example 23. Compound II-13: 7-Diethylamino-3-(5-carboxy-1-phenylbenimidazol-2-yl)coumarin

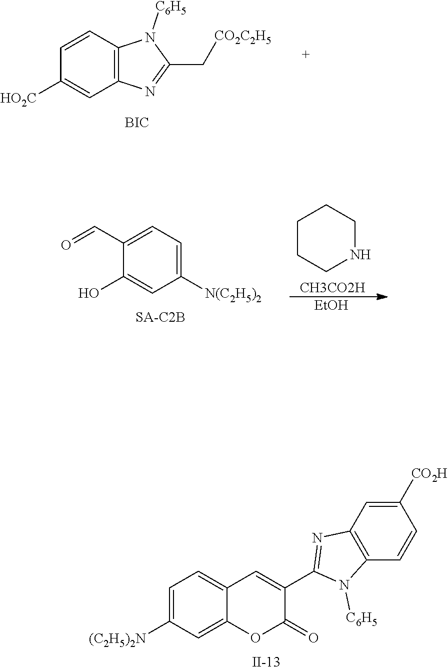

Ethyl (5-carboxy-1-phenylbenimidazol-2-yl)acetate (0.16 g, 1 mmol) and diethylamino salicylic aldehyde (0.21 g, 1.1 mmol) were dissolved in anhydrous ethanol (7 mL). Piperidine (5 drops), and acetic acid (5 drops) were added and the resulting mixture was stirred 5 h at 80° C. and then left at room temperature overnight. The resulting orange precipitate was collected by suction filtration and washed with water. Yield: 0.16 g (70%). Purity, structure and composition of the product were confirmed by HPLC, NMR and LCMS. MS (DUIS): MW Calculated 453.17. Found m/z: (+) 454 (M+1)⁺; (−) 452 (M−1)⁻.

### Example 24. Compound II-14: 3-(5-Carboxybenzoxazol-2-yl)-7-[3-(ethyloxycarbonyl)propyl]amino-coumarin

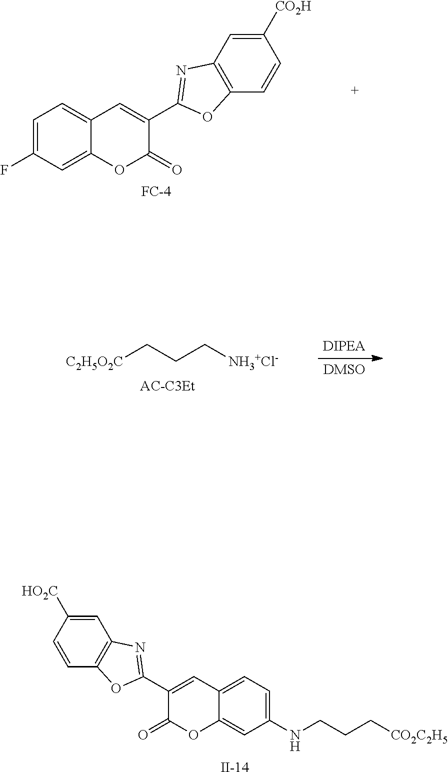

3-(5-Carboxybenzoxazol-2-yl)-7-fluoro-coumarin (0.65 g, 2 mmol) and ethyl 4-aminobutanoate hydrochloride (0.5 g, 3 mmol) were added to anhydrous DMSO (5 mL). After the addition was complete, the mixture was stirred for a few minutes at room temperature and then diisopropylethylamine (0.65 g, 5 mmol) was added. The reaction mixture was stirred for 3 hours at temperature 110° C. After standing at room temperature for 1 hour, the yellow semi-solid reaction mixture was diluted with water (10 mL) and was left stirring overnight. The resulting precipitate was collected by suction filtration. Yield 0.5 g (58%). Purity, structure and composition of the dye were confirmed by HPLC, NMR and LCMS. MS (DUIS): MW Calculated 436.13. Found m/z: (+) 437 (M+1)⁺; (−), 435 (M−1)⁻.

### Example 25. Compound II-15: 3-(6-Carboxybenzoxazol-2-yl)-7-[3-(ethyloxycarbonyl)propyl]amino-coumarin

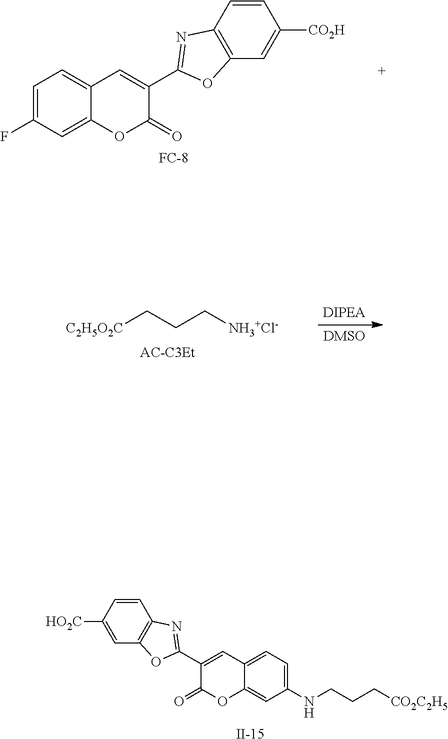

3-(5-Carboxybenzoxazol-2-yl)-7-fluoro-coumarin (0.32 g, 1 mmol) and ethyl 4-aminobutanoate hydrochloride (0.5 g, 3 mmol) were added to anhydrous DMSO (5 mL). After the addition was complete, the mixture was stirred for a few minutes at room temperature and then diisopropylethylamine (0.39 g, 3 mmol) was added. The reaction mixture was stirred for 3 hours at temperature 120° C. After standing at room temperature for 1 hour, the yellow semi-solid reaction mixture was diluted with water (10 mL), acidified with acetic acid (1 mL) and was left stirring overnight. The resulting precipitate was collected by suction filtration. Yield 0.21 g (48%). Purity, structure and composition of the dye were confirmed by HPLC, NMR and LCMS. MS (DUIS): MW Calculated 436.13. Found m/z: (+) 437 (M+1)⁺; (−), 435 (M−1)⁻.

### Example 26. Compound II-16: 7-(3-Carboxypropyl)amino-3-(5-chlorobenzoxazol-2-yl)coumarin

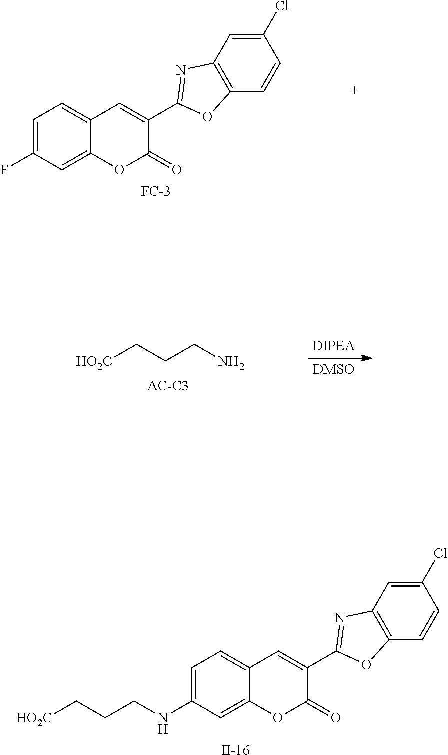

3-(5-Chlorobenzoxazol-2-yl)-7-fluoro-coumarin (0.32 g, 1 mmol) and 4-aminobutanoic acid (0.21 g, 2 mmol) were added to anhydrous DMSO (5 mL) in round bottomed flask. After the addition was complete, the mixture was stirred for a few 20 minutes at room temperature and then diisopropylethylamine (0.52 g, 4 mmol) was added. The reaction mixture was stirred for 7 hours at temperature 135° C. Additional portions of 4-aminobutanoic acid (0.1 g, 1 mmol) and diisopropylethylamine (0.26 g, 2 mmol) were added and heating was continued at 135° C. for 5 hours. After standing at room temperature for 1 hour, the pale-yellow reaction mixture was diluted with water (15 mL) and was stirred overnight. The resulting precipitate was collected by suction filtration. Yield 0.12 g (30%). Purity, structure and composition of the product were confirmed by HPLC, NMR and LCMS. MS (DUIS): MW Calculated 398.07. Found m/z: (+) 399 (M+1)⁺.

### Example 27. Compound II-17: 7-(3-Carboxypropyl)amino-3-(5-benzoxazol-2-yl)coumarin

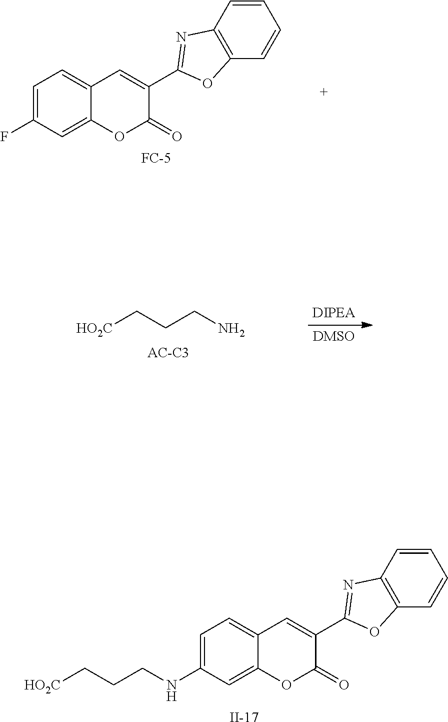

3-(Benzoxazol-2-yl)-7-fluoro-coumarin (0.28 g, 1 mmol) and 4-aminobutanoic acid (0.21 g, 2 mmol) were dissolved in anhydrous DMSO (5 mL) then the mixture was stirred for a few minutes at room temperature and diisopropylethylamine (0.26 g, 2 mmol) was added. The reaction mixture was stirred for 7 hours at temperature 125° C. Additional portions of 4-aminobutanoic acid (0.1 g, 1 mmol) and diisopropylethylamine (0.13 g, 1 mmol) were added and heating was continued at 125° C. for 3 hours. The pale-yellow reaction mixture was diluted with water (10 mL) and was stirred overnight. The resulting precipitate was collected by suction filtration. Yield 0.08 g (23%). Purity, structure and composition of the product were confirmed by HPLC, NMR and LCMS. MS (DUIS): MW Calculated 364.11. Found m/z: (+) 365 (M+1)⁺.

### Example 28. Compound II-18: 3-(5-Carboxybenzoxazol-2-yl)-7-(3-sulfopropyl)amino-coumarin

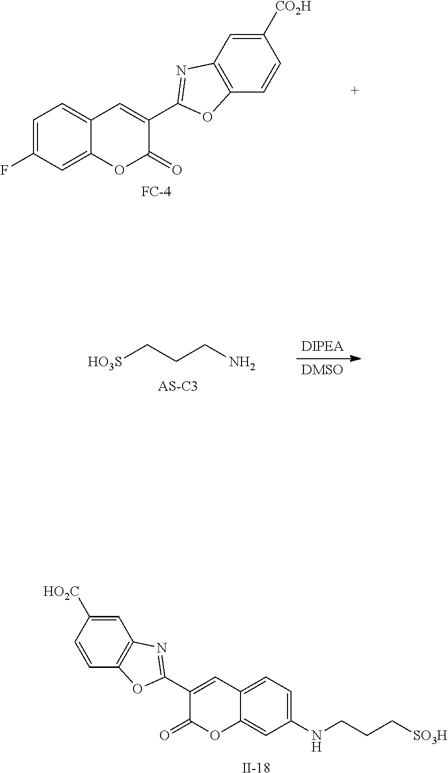

3-(5-Carboxybenzoxazol-2-yl)-7-fluoro-coumarin (0.33 g, 1 mmol) and 3-aminopropansulfonic acid (0.42 g, 3 mmol) were added to anhydrous DMSO (5 mL). After the addition was complete, the mixture was stirred for a few minutes at room temperature and then diisopropylethylamine (0.39 g, 3 mmol) was added. The reaction mixture was stirred for 7 hours at temperature 125° C. A half of the volume of the solvent was distilled off under vacuum. The mixture was left stirred at room temperature for one hour, then was diluted with a water-acetonitrile 1:1 mixture (10 mL), and the product is isolated by preparative HPLC with acetonitrile-TEAB mixture as an eluent. After evaporation of solvents yellow precipitate was triturated with acetonitrile (3 mL) and filtered off Yield: 0.06 g (140%). Purity, structure and composition of the dye were confirmed by HPLC, NMR and LCMS. MS (DIS): MW Calculated 444.06. Found m/z: (+) 445 (M+1).

### Example 29. Comparison of Fluorescence Intensities

Fluorescence intensities of dye solutions (EtOH-water 1:1; at maximum excitation wavelength 450 nm) were compared with a standard dye for the same spectral region. The results are shown in Table 2 and demonstrate significant advantages of the dyes for fluorescence based analytical applications.

TABLE 2
Spectral properties of the fluorescent dyes disclosed in the examples.

| | | | | Relative |
|---|---|---|---|---|
| Compound | | Absorption | Fluorescence | Fluorescence |
| No. | Structure | max (nm) | max (nm) | Intensity (%) |
| II-1 | 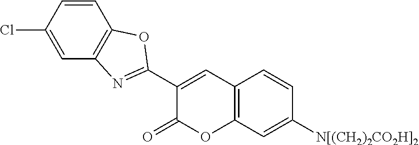 | 458 | 498 | 95 |
| II-2 | 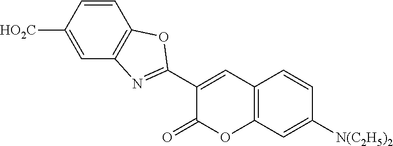 | 455 | 499 | 122 |
| II-3 | 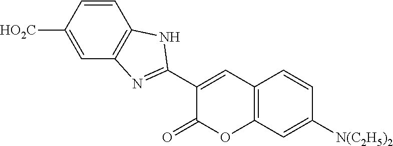 | 443 | 496 | 98 |
| II-4 | 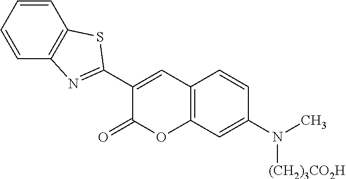 | 470 | 510 | 127 |
| II-5 | | 472 | 510 | 124 |
| II-6 | | 449 | 499 | 125 |

### Example 30. General Procedure for the Synthesis of Fully Functional Nucleotide Conjugates

Coumarin fluorescent dyes disclosed herein were coupled with appropriate amino-substituted adenine (A) and cytosine (C) nucleotide derivatives A-LN3-NH₂ or C-LN3-NH₂:

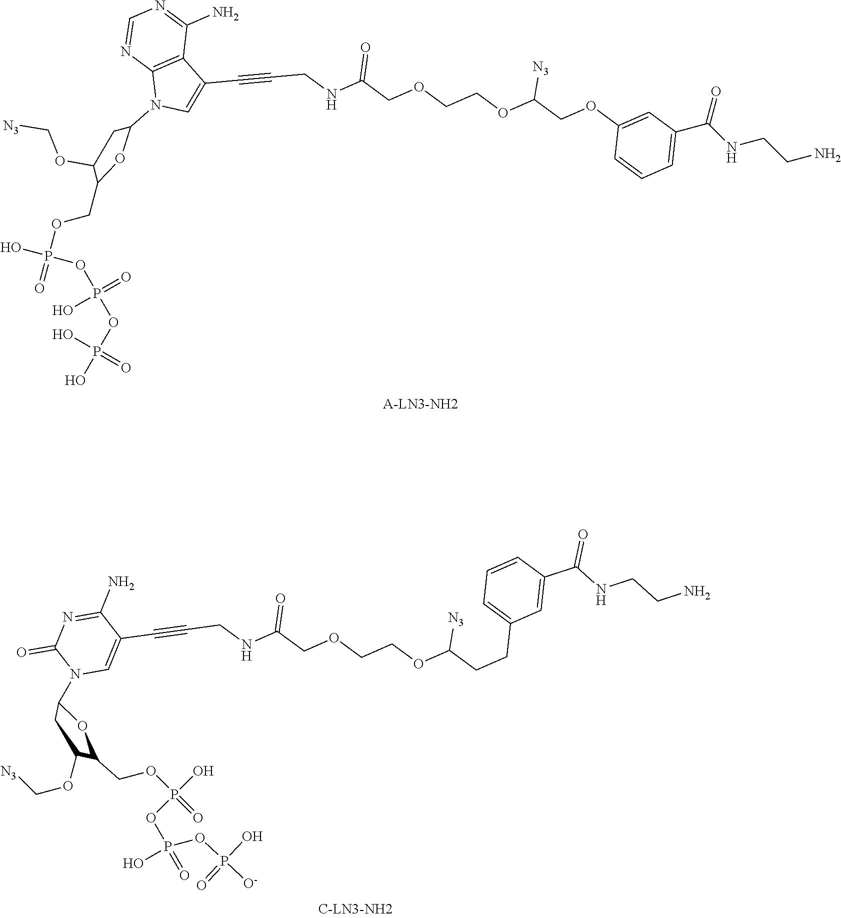

After activation of carboxylic group of a dye with appropriate reagents according to the following adenine scheme:

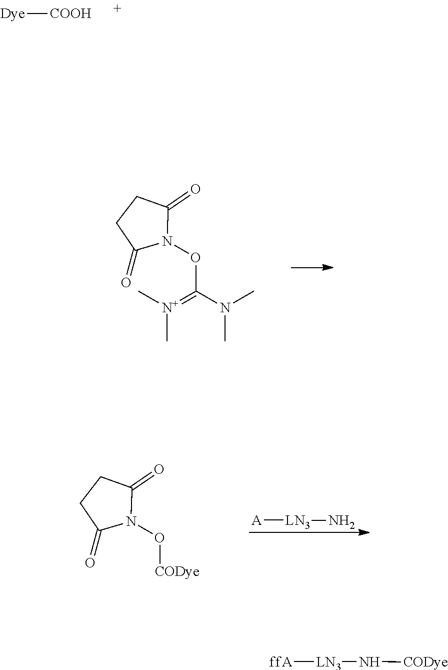

The general product for the adenine coupling is as shown below:

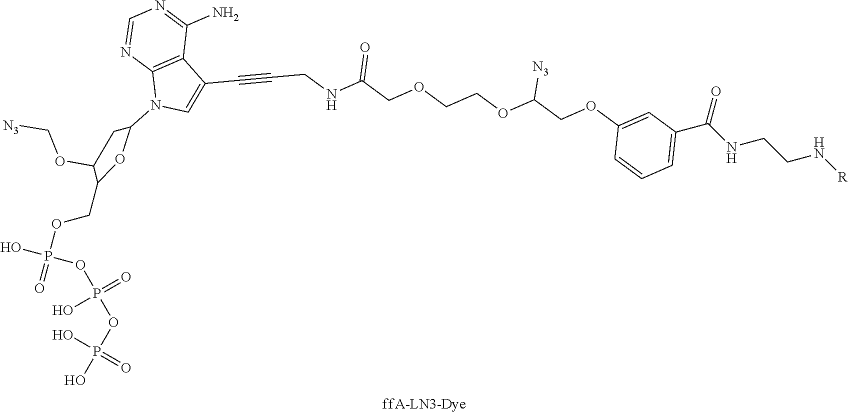

ffA-LN3-Dye refers to a fully functionalized A nucleotide with an LN3 linker and labeled with a coumarin dye disclosed herein. The R group in each of the structures refers to the coumarin dye moiety after conjugation.

The dye (10 μmol) is dried by placing into a 5 mL round-bottomed flask and is dissolved in anhydrous dimethylformamide (DMF, 1 mL) then the solvent is distilled off in vacuo. This procedure is repeated twice. The dried dye is dissolved in anhydrous N,N-dimethylacetamide (DMA, 0.2 mL) at room temperature. N,N,N′,N′-Tetramethyl-O—(N-succinimidyl)uronium tetrafluoroborate (TSTU, 1.5 eq., 15 μmol, 4.5 mg) is added to the dye solution, then DIPEA (3 eq., 30 μmol, 3.8 mg, 5.2 μL) is added via micropipette to this solution. The reaction flask is sealed under nitrogen gas. The reaction progress is monitored by TLC (eluent Acetonitrile-Water 1:9) and HPLC. Meanwhile, a solution of the appropriate amino-substituted nucleotide derivative (A-LN3-NH₂, 20 mM, 1.5 eq, 15 μmol, 0.75 mL) is concentrated in vacuo then re-dissolved in water (20 μL). A solution of the activated dye in DMA is transferred to the flask containing the solution of N-LN3-NH₂. More DIPEA (3 eq, 30 μmol, 3.8 mg, 5.2 μL) is added along with triethylamine (1 μL). Progress of coupling is monitored hourly by TLC, HPLC, and LCMS. When the reaction is complete, triethylamine bicarbonate buffer (TEAB, 0.05 M˜ 3 mL) is added to the reaction mixture via pipette. Initial purification of the fully functionalized nucleotide is carried out by running the quenched reaction mixture through a DEAE-Sephadex® column to remove most of remaining unreacted dye. For example, Sephadex is poured into an empty 25 g Biotage cartridge, solvent system TEAB/MeCN. The solution from the Sephadex column is concentrated in vacuo. The remaining material is redissolved in the minimum volume of water and acetonitrile, before filtering through a 20 μm Nylon filter. The filtered solution is purified by preparative-HPLC. The composition of prepared compounds is confirmed by LCMS.

The general product for the cytosine coupling is as shown below, following similar procedure described above.

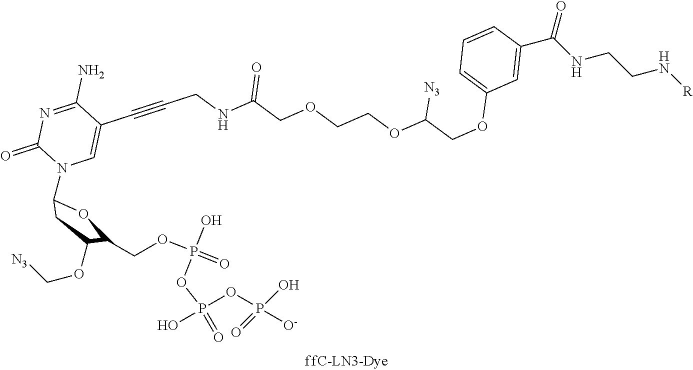

ffC-LN3-Dye refers to a fully functionalized C nucleotide with an LN3 linker and labeled with a coumarin dye disclosed herein. The R group in each of the structures refers to the coumarin dye moiety after conjugation.

### Example 31. Preparation of Amide Derivatives of the Compounds of Formula (II)

Some additional implementations described herein are related to amide derivatives of compounds of Formula (II) and methods of preparing the same, the methods include converting a compound of Formula (IIa) to a compound of Formula (IIa′) through carboxylic acid activation:

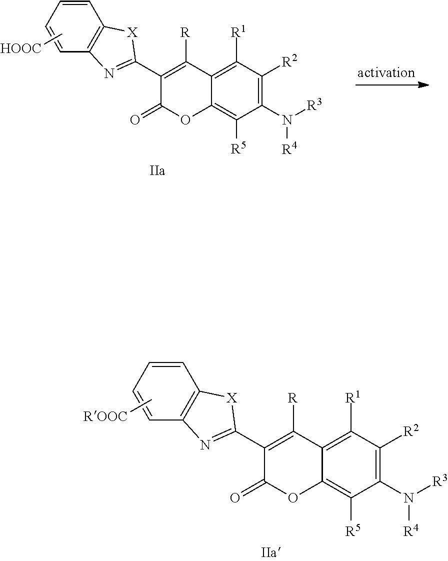

and reacting the compound of Formula (IIa′) with a primary or secondary amine of Formula (Am) to arrive at the amide derivative of Formula (IIb):

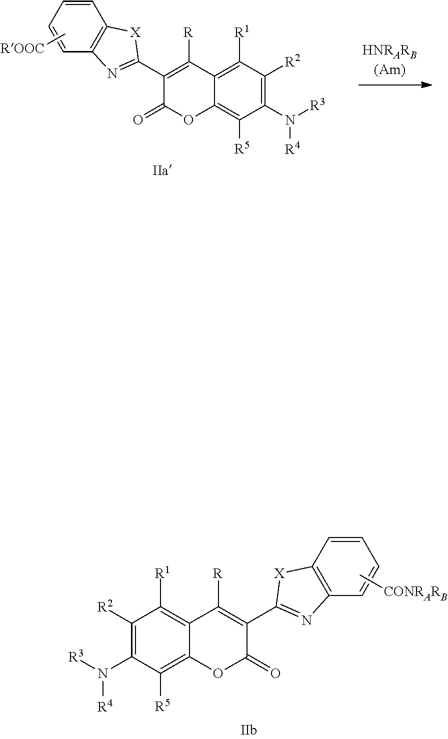

where the variables X, R, R¹, R², R³, R⁴, and R⁵ are defined herein; R′ is the residual moiety of a carboxyl activating agent (such as N-hydroxysuccinimide, nitrophenol, pentafluorophenol, HOBt, BOP, PyBOP, DCC, etc.); each of R_{A} and R_{B} is independently hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, 3-10 membered heterocyclyl, aralkyl, heteroaralkyl, or (heterocyclyl)alkyl.

### General Procedure for the Preparation of Compounds of Formula (IIb)

An appropriate dye of Formula (IIa) (0.001 mol) is dissolved in suitable anhydrous organic solvent (DMF, 1.5 mL). To this solution a carboxyl activating reagent such as TSTU, BOP or PyBOP is added. This reaction mixture is stirred at room temperature for about 20 min and then appropriate amine derivatives is added. The reaction mixture is stirred overnight, filtered and excess of the activation reagent is quenched with 0.1M TEAB solution in water. Solvents is evaporated in vacuum and the residue is re-dissolved in TEAB solution and purified by HPLC.

For example, primary and secondary amide derivatives of Compound II-2 were prepared:

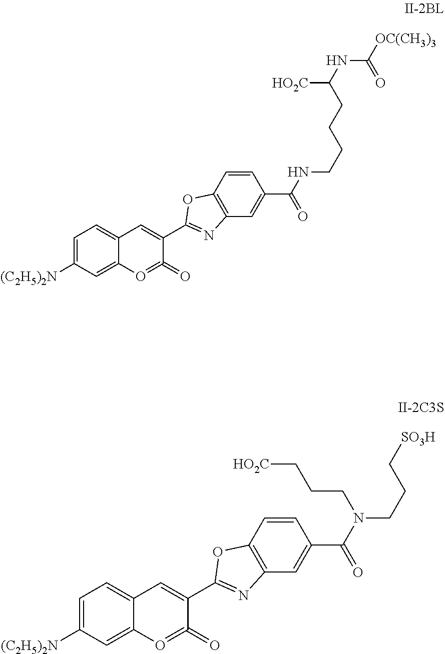

### Example 32. Two-Channel Sequencing Applications

The efficiency of the A nucleotides labeled with the dyes described herein in sequencing application was demonstrated in the two-channel detection method. With respect to the two-channel methods described herein, nucleic acids can be sequenced utilizing methods and systems described in U.S. Patent Application No. 2013/0079232, the disclosure of which is incorporated herein by reference in its entirety.

In the two-channel detection, a nucleic acid can be sequenced by providing a first nucleotide type that is detected in a first channel, a second nucleotide type that is detected in a second channel, a third nucleotide type that is detected in both—the first and the second channel and a fourth nucleotide type that lacks a label that is not, or minimally, detected in either channel. The scatterplots were generated by RTA2.0.93 analysis of an experiment. The scatterplot illustrated in Figure below was at cycle 5 of each of the 26 cycle runs.

Sequencing Conditions:

Scanning at 60 C, Pol1671, on CCL FCs (cluster chemical linearization), PhiX

Green dye as follow except for set 3: ffA-BL-NR550S0/ffT-AF550POPOS0
Isothermal Sequencing 2×151c
   Scanning at 60° C., Pol1671, on CCL FCs (cluster chemical linerisation), PhiX
   Green dye as follow except for set 3: ffA-BL-NR55050/ffT-AFSS0POPCS0

| Set | | Blue exp [ms] | Green exp [ms] | P/PP R1 | P/PP R2 | ER% R1/R2 |
|---|---|---|---|---|---|---|
| 1 | A-LN3-BLNR450H/C- | 250 | 1000 | 0.167/0.132 | 0.182/0.139 | 0.38/0.55 |
| | sPA-LN3-NR455Boc | | | | | |
| 2 | A-BLNR450H/C- | 250 | 1000 | 0.073/0.197 | 0.085/0.202 | 0.74/0.73 |
| | sPA-LN3-NR442C35 | | | | | |
| 3 | T-LN3-NR550S0/ | 250 | 500 | 0.77/0.150 | 0.198/0.155 | 0.48/0.80 |
| | A-7180A/A-BLNR450H/C- | | | | | |
| | sPA-LN3-NR455Boc | | | | | |
| 4 | A-LN3-BL-NR455Boc/C- | 500 | 500 | 0.191/0.136 | 0.170/0.137 | 0.52/0.71 |
| | sPA-LN3-NR440 | | | | | |
| 5 | A-LN3-BL-NR455Boc/C- | 500 | 500 | 0.092/0.155 | 0.105/0.157 | 0.38/0.48 |
| | sPA-LN3-NR430ClC3S | | | | | |

### Scatterplot Figure

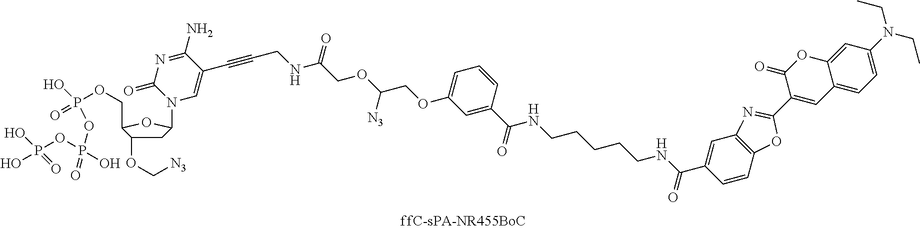 [SMILES: CCN(CC)c1ccc2cc(-c3nc4cc(C(=O)NCCCCCNC(=O)c5cccc(OCC(N=[N+]=[N-])OCC(=O)NCC#Cc6cn(C7CC(OCN=[N+]=[N-])C(COP(=O)(O)OP(=O)(O)OP(=O)(O)O)O7)c(=O)nc6N)c5)ccc4o3)c(=O)oc2c1]

In some implementations, secondary amine-substituted coumarin compounds may be particularly suitable for methods of fluorescence detection and sequencing by synthesis. Implementations described herein relate to dyes and their derivatives of the structure of Formula (III) or salts thereof:

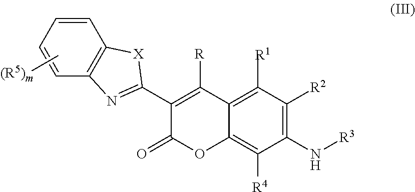

wherein:
X is O, S, Se, or NRⁿ, where Rⁿ is H or C₁₋₆alkyl;
R and R¹ are each independently H, halo, —CN, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl;
R² and R⁴ are each independently H, halo, —CN, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl; or one of R² and R⁴ is linked to R³ to form an optionally substituted heterocyclic ring;
R³ is H, C₁₋₆alkyl, substituted C₂-alkyl, optionally substituted C₂₋₆alkenyl, optionally substituted C₂₋₆alkynyl, or optionally substituted carbocyclyl, heterocyclyl, aryl, or heteroaryl, or R³ is linked to R² or R⁴ to form an optionally substituted ring; wherein when R is —CN, R³ is not C₁₋₆alkyl;
each R⁵ is independently halo, —CN, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl; and
m is 0, 1, 2, 3, or 4.

In some aspects, R is not —CN, such that R is H, halo, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl.

In another aspect is a compound of Formula (IV) or a salt thereof:

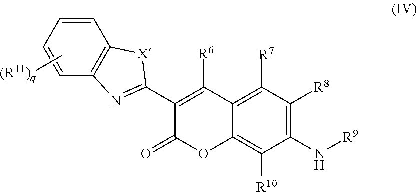

wherein:
X′ is selected from O, S, and NR, where R′ is H or C₁₋₆alkyl;
R⁶ is H or C₁₋₄alkyl;
R⁷ is H, halo, —CN, —OH, optionally substituted C₁₋₄alkyl, optionally substituted C₁₋₄ alkenyl, optionally substituted C₂₋₄alkynyl, —CO₂H, —SO₃H, —SO₂NH₂, —SO₂NH(C₁₋₄ alkyl), —SO₂N(C₁₋₄alkyl)₂, and optionally substituted C₁₋₄alkoxy;
R⁸ and R¹⁰ are each independently H, halo, —CN, —CO₂H, amino, —OH, —SO₃H, —SO₂NH₂, —SO₂NH(C₁₋₄alkyl), —SO₂N(C₁₋₄alkyl)₂, optionally substituted C₁₋₆ alkyl, optionally substituted C₁₋₆alkenyl, optionally substituted C₂₋₆alkynyl, or optionally substituted C₁₋₆alkoxy; or
one of R⁸ and R¹⁰ is H, halo, —CN, —CO₂H, amino, —OH, —SO₃H, —SO₂NH₂, —SO₂NH(C₁₋₄ alkyl), —SO₂N(C₁₋₄alkyl)₂, optionally substituted C₁₋₆alkyl, optionally substituted C₁₋₆ alkenyl, optionally substituted C₂₋₆alkynyl, or optionally substituted C₁₋₆alkoxy, and the other of R⁸ and R¹⁰ is taken with R⁹ to form an optionally substituted 4- to 7-membered heterocyclic ring;
R⁹ is C₂₋₆alkyl or C₁₋₆alkyl substituted with —CO₂H, —CO₂C₁₋₄alkyl, —CONH₂, —CONH(C₁₋₄ alkyl), —CON(C₁₋₄alkyl)₂, —CN, —SO₃H, —SO₂NH₂, —SO₂NH(C₁₋₄alkyl), or —SO₂N(C₁₋₄ alkyl)₂;
each R¹¹ is independently halo, —CN, carboxy, amino, —OH, C-amido, N-amido, nitro, —SO₃H, —SO₂NH₂, —SO₂NH(C₁₋₄alkyl), —SO₂N(C₁₋₄alkyl)₂, optionally substituted alkyl, optionally substituted alkenyl, optionally substituted alkynyl, and optionally substituted C₁₋₆alkoxy; and
q is 0, 1, or 2.

Regarding compounds of Formula (III) or salts thereof, particular implementations for the various substituents are shown below. Each single group can be combined with any other individual limitation unless otherwise specified.

To improve fluorescent properties of the biomarkers and especially their bioconjugates in water-based solutions, the compound of Formula (III) is a compound in which:

i) R² is —SO₃H; and/or

ii) R⁴ is —SO₃H; and/or

iii) R⁵ is —SO₃H or —SO₂NH₂.

In some aspects, X is O or S. In some aspects, X is O. In some aspects, X is S. In some aspects, X is NRⁿ, where Rⁿ is H or C₁₋₆alkyl, and in some aspects, Rⁿ is H.

In some aspects, R³ is H. In some aspects, R³ is methyl, ethyl, propyl, isopropyl, butyl, isobutyl, sec-butyl, tert-butyl, pentyl, or hexyl. In other aspects, R³ is ethyl. In other aspects, R³ is substituted C₂₋₆alkyl. In other aspects, R³ is C₂₋₆alkyl substituted with —CO₂H. In other aspects, R³ is optionally substituted C₂₋₆alkenyl or optionally substituted C₂₋₆alkynyl. In some aspects, R³ is linked to R² or R⁴ to form an optionally substituted ring.

Where coupling to a linker or nucleotide is via R³, R³ should be of sufficient length to allow coupling to a functional group attached thereto. In some aspects, R³ is not —CH₂COOH or —CH₂COO⁻.

Optionally, R³ is —(CH₂)˜COOH where n is 2-6. In some aspects, n is 2, 3, 4, 5 or 6. In other aspects, n is 2 or 5. In some aspects, n is 2. In some aspects, n is 5.

Optionally, R³ is —(CH₂)˜ SO₃H where n is 2-6. In some aspects, n is 2, 3, 4, 5 or 6. In other aspects, n is 2 or 5. In some aspects, n is 2. In some aspects, n is 5.

The benzene ring of the indole moiety is optionally substituted in any one, two, three, or four positions by a substituent shown as R⁵. Where m is zero, the benzene ring is unsubstituted. Where m is greater than 1, each R⁵ may be the same or different. In some aspects, m is 0. In other aspects, m is 1. In other aspects, m is 2. In some aspects, m is 1, 2, or 3, and each R⁵ is independently halo, —CN, —CO₂H, amino, —OH, —SO₃H, or —SO₂NH₂. In some aspects, R⁵ is —(CH₂)ₓCOOH where x is 2-6. In some aspects, x is 2, 3, 4, 5 or 6. In other aspects, x is 2 or 5. In some aspects, x is 2. In some aspects, x is 5.

In some aspects, R⁵ is halo, —CN, —CO₂H, —SO₃H, —SO₂NH₂, or optionally substituted C₁₋₆alkyl. In some aspects, R⁵ is halo, —CO₂H, —SO₃H, or —SO₂NH₂. In some aspects, R⁵ is C₂₋₆alkyl substituted with —CO₂H, —SO₃H, or —SO₂NH₂. In some aspects, each R⁵ is independently optionally substituted C₁₋₆alkyl, halo, —CN, —CO₂H, amino, —OH, —SO₃H, or —SO₂NH₂.

In some aspects, R¹ is H. In some aspects, R¹ is halo. In some aspects, R¹ is Cl. In some aspects, R¹ is C₁₋₆alkyl. In some aspects, R¹ is methyl.

In some aspects, R is H. In some aspects, R is halo. In some aspects, R is Cl. In some aspects, R is C₁₋₆alkyl. In some aspects, R is methyl. In some aspects, R is not —CN. In some aspects, R is H, halo, —CO₂H, amino, —OH, C-amido, N-amido, —NO₂, —SO₃H, —SO₂NH₂, optionally substituted alkyl, optionally substituted alkenyl, optionally substituted alkynyl, optionally substituted alkoxy, optionally substituted aminoalkyl, optionally substituted carbocyclyl, optionally substituted heterocyclyl, optionally substituted aryl, or optionally substituted heteroaryl.

In some aspects, R² is H. In some aspects, R² is optionally substituted alkyl. In some aspects, R² is C₁₋₄alkyl optionally substituted with —CO₂H or —SO₃H. In some aspects, R² is —SO₃H. In some aspects, R² is linked to R³ to form an optionally substituted heterocyclic ring, such as a pyrrolidine or piperidine, optionally substituted with one or more alkyl groups. In some aspects, R² is H, optionally substituted alkyl, C₁₋₄alkyl optionally substituted with —CO₂H or —SO₃H, or —SO₃H. In some aspects, R² is H or —SO₃H.

In some aspects, R⁴ is H. In some aspects, R⁴ is optionally substituted alkyl. In some aspects, R⁴ is C₁₋₄alkyl optionally substituted with —CO₂H or —SO₃H. In some aspects, R⁴ is —SO₃H. In some aspects, R⁴ is linked to R³ to form an optionally substituted heterocyclic ring, such as a pyrrolidine or piperidine, optionally substituted with one or more alkyl groups.

Particular examples of a compound of Formula (III) include where X is O or S; R is H; R¹ is H; R³ is —(CH₂)˜COOH where n is 2-6; R⁵ is H, —SO₃H, or —SO₂NH₂; R² is H or —SO₃H; and R⁴ is H or —SO₃H.

Particular examples of a compound of Formula (III) include where X is O or S; R is H; R¹ is H; R³ is —(CH₂)₂COOH; R⁵ is H, —SO₃H, or —SO₂NH₂; R² is H or —SO₃H; and R⁴ is H or —SO₃H.

Particular examples of a compound of Formula (III) include where X is O or S; R is H; R¹ is H; R³ is —(CH₂)₅COOH; R⁵ is H, —SO₃H, or —SO₂NH₂; R² is H or —SO₃H; and R⁴ is H or —SO₃H.

In some aspects of Formula (IV), X′ is O. In some aspects, X′ is S. In some aspects, X′ is NR, where RP is H or C₁₋₆alkyl. In some aspects, X′ is NR, where RP is H.

In some aspects, R⁶ is H. In some aspects, R⁶ is C₁₋₄alkyl.

In some aspects, R⁷ is H. In some aspects, R⁷ is optionally substituted C₁₋₄alkyl, —CO₂H, —SO₃H, —SO₂NH₂, —SO₂NH(C₁₋₄alkyl), or —SO₂N(C₁₋₄alkyl)₂. In some aspects, R⁷ is C₁₋₄alkyl optionally substituted with —CO₂H.

In some aspects, R⁸ is H. In some aspects, R⁸ is —CO₂H, —SO₃H, or —SO₂NH₂. In some aspects, R⁸ is —SO₃H.

In some aspects, R¹⁰ is H. In some aspects, R¹⁰ is —CO₂H, —SO₃H, or —SO₂NH₂. In some aspects, R¹⁰ is —SO₃H. In some aspects, R⁸ is H and R¹⁰ is —SO₃H. In some aspects, R⁸ is —SO₃H and R¹⁰ is H.

In some aspects, one of R⁸ and R¹⁰ is H, halo, —CN, —CO₂H, amino, —OH, —SO₃H, —SO₂NH₂, —SO₂NH(C₁₋₄alkyl), —SO₂N(C₁₋₄alkyl)₂, optionally substituted C₁₋₆alkyl, optionally substituted C₁₋₆alkenyl, optionally substituted C₂₋₆alkynyl, or optionally substituted C₁₋₆alkoxy, and the other of R⁸ and R¹⁰ is taken with R⁹ to form an optionally substituted 4- to 7-membered heterocyclic ring.

In some aspects, R⁹ is C₂₋₆alkyl. In some aspects, R⁹ is C₁₋₆alkyl substituted with —CO₂H, —CO₂C₁₋₄alkyl, —CONH₂, —CONH(C₁₋₄alkyl), —CON(C₁₋₄alkyl)₂, —CN, —SO₃H, —SO₂NH₂, —SO₂NH(C₁₋₄alkyl), or —SO₂N(C₁₋₄alkyl)₂. In some aspects, R⁹ is C₁₋₆alkyl substituted with —CO₂H. In some aspects, R⁹ is —(CH₂)_{y}—CO₂H, where y is 2, 3, 4, or 5.

In some aspects, each R¹¹ is independently halo, —CO₂H, —SO₃H, —SO₂NH₂, —SO₂NH(C₁₋₄alkyl), —SO₂N(C₁₋₄alkyl)₂, or optionally substituted alkyl. In other aspects, each R¹¹ is independently halo, —CO₂H, —SO₃H, or —SO₂NH₂.

In some aspects, q is 0. In other aspects, q is 1. In still other aspects, q is 2.

Specific examples of secondary amine-substituted coumarin dyes include:

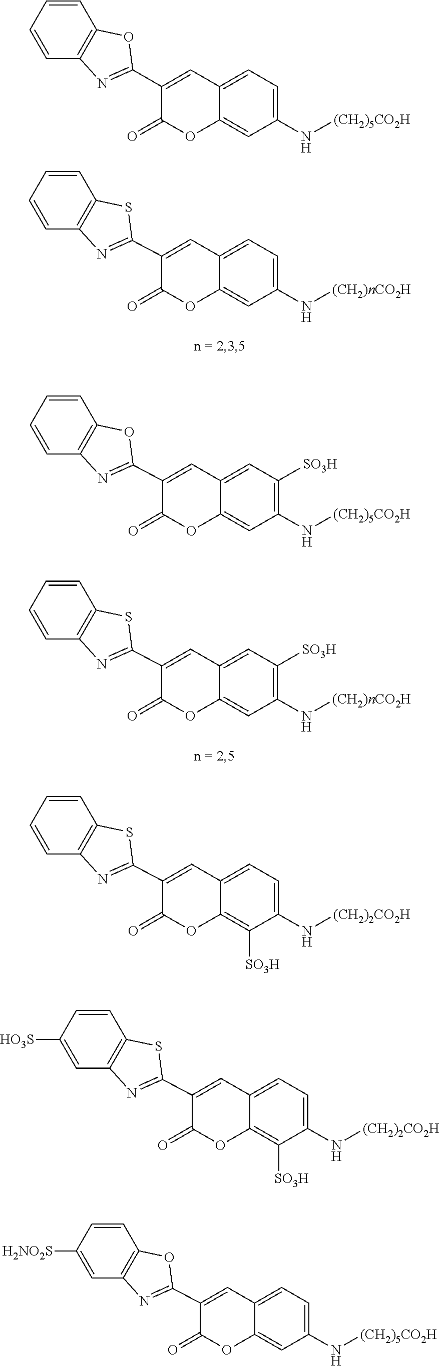

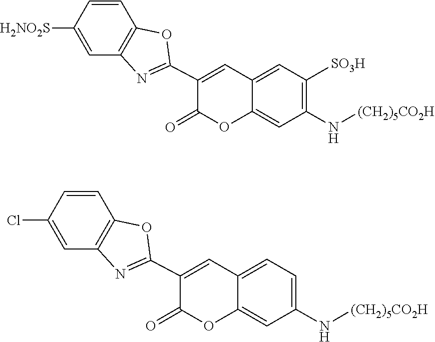

and salts thereof.

A particularly useful compound is a nucleotide or oligonucleotide labeled with a dye as described herein. The labeled nucleotide or oligonucleotide may have the label attached to the nitrogen atom of coumarin molecule via an alkyl-carboxy group to form an alkyl-amide. The labeled nucleotide or oligonucleotide may have the label attached to the C5 position of a pyrimidine base or the C7 position of a 7-deaza purine base through a linker moiety.

The labeled nucleotide or oligonucleotide may also have a blocking group covalently attached to the ribose or deoxyribose sugar of the nucleotide. The blocking group may be attached at any position on the ribose or deoxyribose sugar. In particular implementations, the blocking group is at the 3′ OH position of the ribose or deoxyribose sugar of the nucleotide.

Provided herein are kits including two or more nucleotides wherein at least one nucleotide is a nucleotide labeled with a compound of the present disclosure. The kit may include two or more labeled nucleotides. The nucleotides may be labeled with two or more fluorescent labels. Two or more of the labels may be excited using a single excitation source, which may be a laser. For example, the excitation bands for the two or more labels may be at least partially overlapping such that excitation in the overlap region of the spectrum causes both labels to emit fluorescence. In particular implementations, the emission from the two or more labels will occur in different regions of the spectrum such that presence of at least one of the labels can be determined by optically distinguishing the emission.

The kit may contain four labeled nucleotides, where the first of four nucleotides is labeled with a compound as disclosed herein. In such a kit, each of the four nucleotides can be labeled with a compound that is the same or different from the label on the other three nucleotides. Thus, one or more of the compounds can have a distinct absorbance maximum and/or emission maximum such that the compound(s) is(are) distinguishable from other compounds. For example, each compound can have a distinct absorbance maximum and/or emission maximum such that each of the compounds is distinguishable from the other three compounds. It will be understood that parts of the absorbance spectrum and/or emission spectrum other than the maxima can differ and these differences can be exploited to distinguish the compounds. The kit may be such that two or more of the compounds have a distinct absorbance maximum. The compounds may absorb light in the region below 500 nm.

The compounds, nucleotides, or kits that are set forth herein may be used to detect, measure, or identify a biological system (including, for example, processes or components thereof). Some techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two lasers operating at different wavelengths.

Disclosed herein are methods of synthesizing compounds of the disclosure. Dyes according to the present disclosure may be synthesized from a variety of different suitable starting materials. Methods for preparing coumarin dyes are well known in the art.

Compounds described herein can be represented as several mesomeric forms. Where a single structure is drawn, any of the relevant mesomeric forms are intended. The coumarin compounds described herein are represented by a single structure but can equally be shown as any of the related mesomeric forms. Some mesomeric structures are shown below for Formula (III):

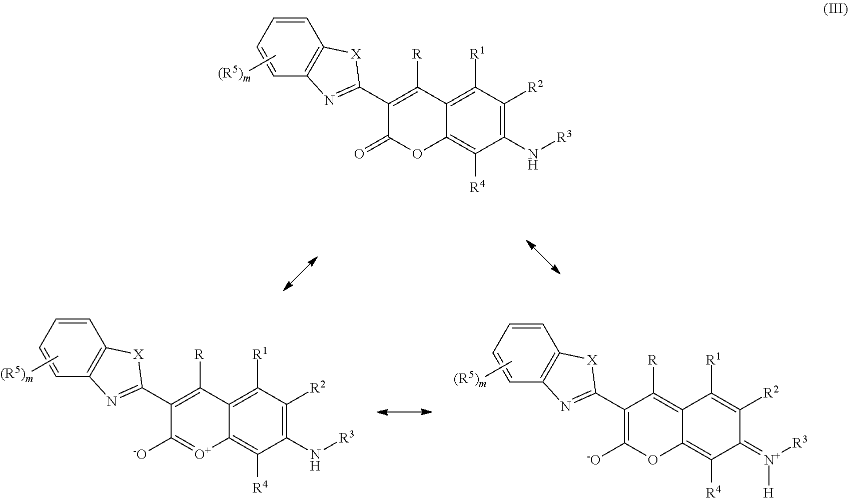

In each instance where a single mesomeric form of a compound described herein is shown, the alternative mesomeric forms are equally contemplated.

The attachment to the biomolecules may be via the R, R¹, R², R³, R⁴, R⁵, or X position of the compound of Formula (III). In some aspects, the connection is via the R³ or R⁵ group of Formula (III). For Formula (IV), attachment may be at any position R⁶⁻¹¹ or X′. In some implementations, the substituent group is a substituted alkyl, for example, alkyl substituted with —CO₂H or an activated form of carboxyl group, for example, an amide or ester, which may be used for attachment to the amino or hydroxyl group of the biomolecules. In one implementation, the R, R¹, R², R³, R⁴, R⁵, or X group of Formula (III) or the R⁶⁻¹¹ or X′ groups of Formula (IV) may contain an activated ester or amide residue most suitable for further amide/peptide bond formation. The term “activated ester” as used herein, refers to a carboxyl group derivative which is capable of reacting in mild conditions, for example, with a compound containing an amino group. Non-limiting examples of activated esters include but not limited to p-nitrophenyl, pentafluorophenyl and succinimido esters.

In some implementations, the dye compounds may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a linker moiety. The labeled nucleotide or oligonucleotide may also have a 3-OH blocking group covalently attached to the ribose or deoxyribose sugar of the nucleotide.

A particular useful application of the fluorescent dyes as described herein is for labeling biomolecules, for example, nucleotides or oligonucleotides. Some implementations of the present application are directed to a nucleotide or oligonucleotide labeled with the fluorescent compounds as described herein.

Additional implementations are disclosed in further detail in the following examples, which are not in any way intended to limit the scope of the claims.

Additional implementations are disclosed in further detail in the following examples, which are not in any way intended to limit the scope of the claims. Table 3 summarizes spectral properties of the coumarin fluorescent dyes disclosed in the examples. Table 4 summarizes the structure and spectral properties of various nucleotides labeled with dyes disclosed herein.

### Example 33: Compound III-1-1: 7-(5-Carboxypentyl)amino-3-(benzothiazol-2-yl)coumarin

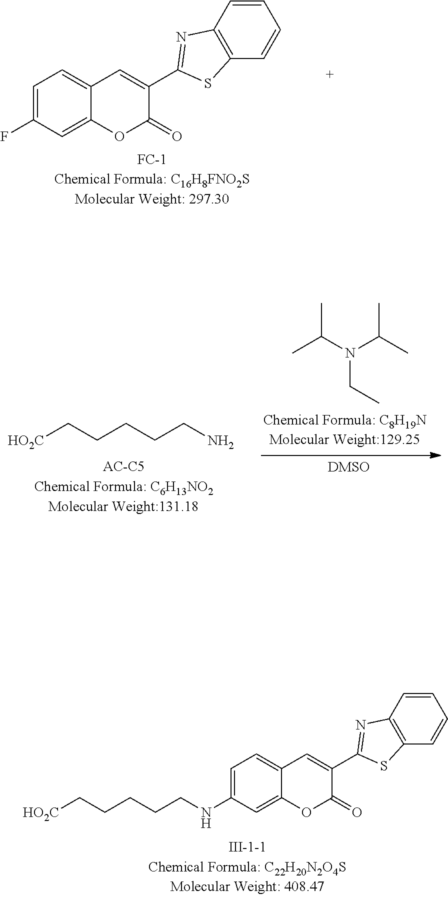

3-(Benzothiazol-2-yl)-7-fluoro-coumarin derivative (FC-1, 0.4 g, 1.345 mmol, 1 eqv) and 6-aminohexanoic acid (AC-C5, 0.25 g, 1.906 mmol, 1.417 eqv) was added to anhydrous dimethyl sulfoxide (DMSO, 3 mL). After the addition was complete, the mixture was stirred for a few minutes at room temperature and then N,N-diisopropyl-N-ethylamine (DIPEA, 0.25 g, 2 mmol, 2 eqv) was added to this mixture. The reaction mixture was stirred for 3 hours at 120° C. After standing at room temperature for 1 hour, the yellow, semi-solid reaction mixture was diluted with water (5 mL) and stirred overnight. The resulting precipitate was collected by suction filtration. Yield 0.36 g (65.5%). MS (DUIS): MW Calculated 408.47. Found m/z: (+) 409 (M+1)⁺; (−), 407 (M−1)⁻. ¹H NMR (400 MHz, DMSO-d₆) δ:12.03 (m, 2H), 9.00 (s, 1H), 8.12 (d, J=7.9 Hz, 1H), 7.99 (d, J=8.1 Hz, 1H), 6.73 (dd, J=8.8, 2.1 Hz, 1H), 6.54 (d, J=2.0 Hz, 1H), 3.18 (q, J=6.5 Hz, 2H), 2.23 (t, J=7.3 Hz, 2H), 1.57 (dp, J=14.7, 7.2 Hz, 4H), 1.39 (dq, J=9.2, 4.5, 3.5 Hz, 2H).

### Example 34: Compound III-1-2: 7-(5-Carboxypentyl)amino-3-(benzimidazol-2-yl)coumarin

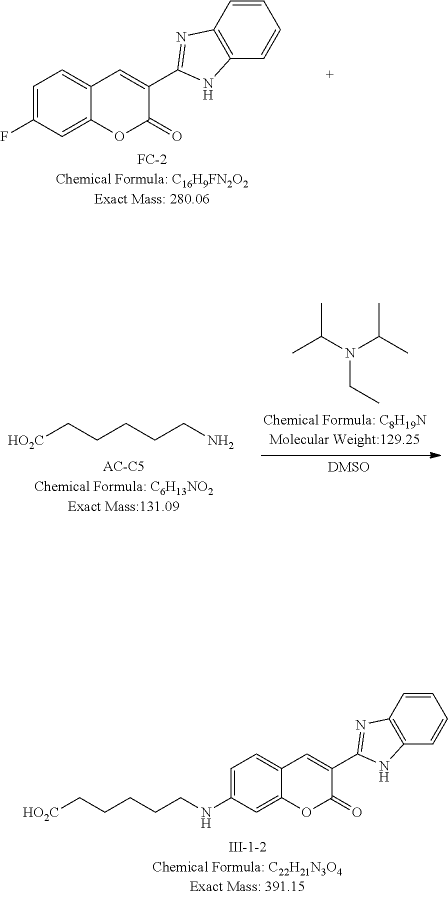

3-(Benzimidazol-2-yl)-7-fluoro-coumarin (FC-2, 0.28 g, 1 mmol, 1 eqv) and 6-aminohexanoic acid (AC-C5, 0.13 g, 1 mmol, 1 eqv) was added to anhydrous dimethyl sulfoxide (DMSO, 2 mL). The resulting mixture was stirred for a few minutes at room temperature and then DIPEA (0.25 g, 2 mmol, 2 eqv) was added. The reaction mixture was stirred for 4 hours at temperature 130° C. Additional portions of 6-aminohexanoic acid (AC-1, 0.13 g, 1 mmol, 1 eqv) and DIPEA (0.26 g, 2 mmol, 2 eqv) was added to the reaction mixture and heating was continued at 130° C. was continued for 5 hours. After standing at room temperature for 1 hour, the pale-yellow reaction mixture was diluted with water (5 mL) and stirred overnight. The resulting precipitate was collected by suction filtration. Yield 0.26 g (68.5%). Purity, structure and composition of the product were confirmed by HPLC, NMR and LCMS. MS (DUIS): MW Calculated 391.15. Found m/z: (+) 392 (M+1)⁺; (−) 390 (M−1), 781 (2M−1)⁻.

### Example 35: Compound III-1-3: 7-(2-Carboxyethyl)amino-3-(benzothiazol-2-yl)coumarin

### Step A: 7-[2-(t-Butyloxycarbonyl)ethyl]amino-3-(benzothiazol-2-yl)coumarin

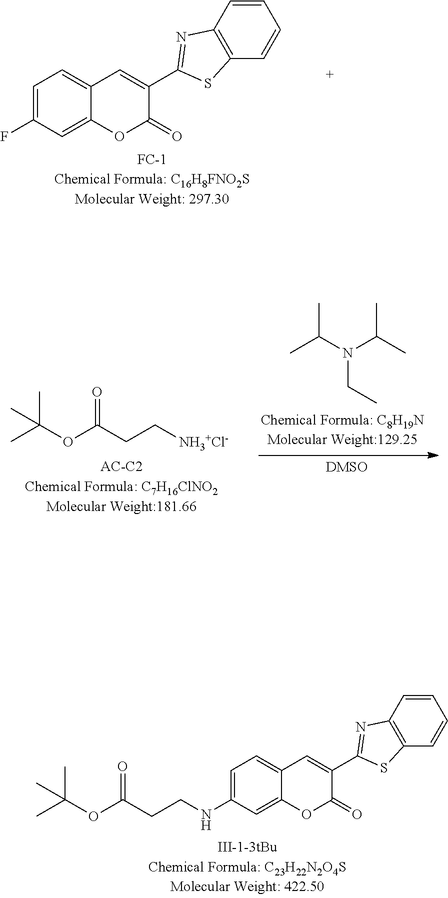

3-(Benzothiazol-2-yl)-7-fluoro-coumarin (FC-1, 0.3 g, 1.01 mmol, 1 eqv) and t-butyl 3-aminopropionate hydrochloride (AC-C2, 0.2 g, 1.1 mmol, 1.09 eqv) was added to anhydrous dimethyl sulfoxide (DMSO, 2 mL) and the resulting mixture was stirred for a few minutes at room temperature and then DIPEA (0.26 g, 2 mmol, 2 eqv) was added. The resulting mixture was stirred for 2 hours at 100° C. After standing at room temperature for 1 hour, the yellow reaction mixture was diluted with water (7 mL) and was stirred overnight. The resulting precipitate was collected by suction filtration. Yield 0.38 g (69%). MS (DUIS): MW Calculated 422.13. Found m/z: (+) 423 (M+1)⁺; (−), 421 (M−1)⁻. ¹H NMR (400 MHz, DMSO-d₆) δ: 9.28 (s, 1H), 9.01 (s, 1H), 8.27-8.16 (m, 1H), 8.10 (tt, J=8.3, 0.9 Hz, 2H), 8.05-7.92 (m, 1H), 7.72 (d, J=8.8 Hz, 1H), 7.66-7.55 (m, 1H), 7.51 (dddd, J=11.4, 8.2, 7.1, 1.3 Hz, 2H), 7.46-7.32 (m, 2H), 6.74 (dd, J=8.7, 2.1 Hz, 1H), 6.58 (d, J=2.1 Hz, 1H), 3.41 (q, J=6.3 Hz, 2H), 2.55 (t, J=6.4 Hz, 2H), 1.41 (s, 9H).

### Step B

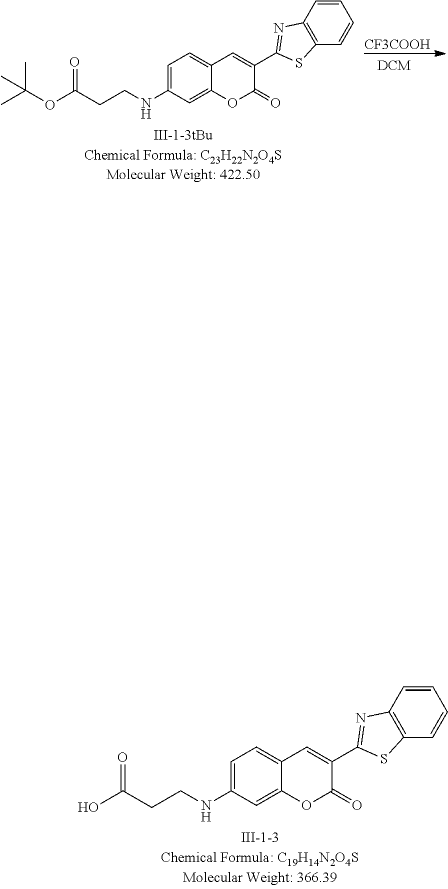

A solution of 7-[2-(t-butyloxycarbonyl)ethyl]amino-3-(benzothiazol-2-yl)coumarin (III-1-3tBu, 0.2 g, 0.473 mmol) in anhydrous dichloromethane (20 mL) was treated with trifluoroacetic acid (0.5 mL) and the resulting mixture was stirred for 24 hours at room temperature. The solvents were distilled off and the residue was triturated with water (10 mL). The resulting precipitate was collected by suction filtration. Yield 0.15 g (86%). Purity, structure and composition were confirmed by HPLC, NMR and LCMS. MS (DUIS): MW Calculated 366.39. Found m/z: (+) 367 (M+1)⁺; (−), 365 (M−1)⁻.

### Example 36: Compound III-1-4: 7-(3-Carboxypropyl)amino-3-(benzothiazol-2-yl)coumarin

### Step A: 7-[3-(t-Butyloxycarbonyl)propyl]amino-3-(benzothiazol-2-yl)coumarin

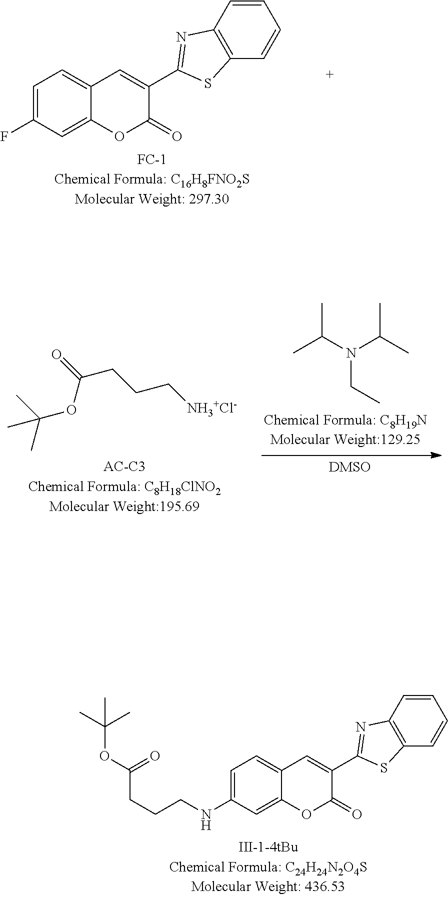

3-(Benzothiazol-2-yl)-7-fluoro-coumarin (FC-1, 0.6 g, 2.02 mmol, 1 eqv) and t-butyl 4-aminobutanoate hydrochloride (AC-C3, 0.5 g, 2.56 mmol, 1.27 eqv) were added to anhydrous dimethyl sulfoxide (DMSO, 5 mL). After the addition was complete, the mixture was stirred for a few minutes at room temperature and then DIPEA (0.65 g, 5 mmol, 4 eqv) was added. The reaction mixture was stirred for 3 hours at temperature 100° C. After standing at room temperature for 1 hour, the yellow semi-solid reaction mixture was diluted with water (10 mL) and was left stirring overnight. The resulting precipitate was collected by suction filtration. Yield 0.7 g (79%). Purity, structure and composition were confirmed by HPLC, NMR and LCMS. MS (DUIS): MW Calculated 436.53. Found m/z: (+) 437 (M+1)⁺; (−), 435 (M−1)⁻.

### Step B

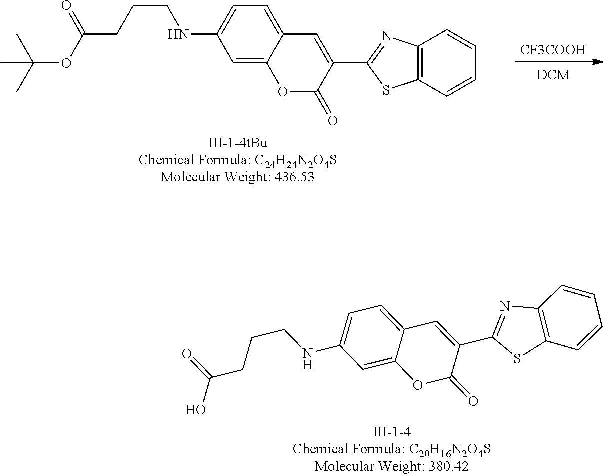

A solution of 7-[3-(t-Butyloxycarbonyl)propyl]amino-3-(benzothiazol-2-yl)coumarin (III-1-4tBu, 0.7 g, 1.604 mmol) in anhydrous dichloromethane (25 mL) was treated with trifluoroacetic acid (1 mL) and the reaction mixture was stirred for 24 hours at room temperature. The solvents were distilled off and the residue was triturated with water (10 mL). The resulting precipitate was collected by suction filtration. Yield 0.59 g (97%). Purity, structure and composition were confirmed by HPLC, NMR and LCMS. MS (DUIS): MW Calculated 366.39. Found m/z: (+) 381 (M+1)⁺; (−), 379 (M−1)⁻. ¹H NMR (400 MHz, DMSO-d₆) δ: 12.17 (s, 1H), 9.01 (s, 1H), 8.12 (d, J=8.0 Hz, 1H), 7.99 (d, J=8.1 Hz, 1H), 7.71 (d, J=8.8 Hz, 1H), 7.48-7.30 (m, 2H), 6.73 (dd, J=8.8, 2.1 Hz, 1H), 6.57 (d, J=2.1 Hz, 1H), 3.21 (q, J=6.6 Hz, 2H), 2.36 (d, J=7.3 Hz, 2H), 1.80 (p, J=7.3 Hz, 2H).

### Example 37: Compound III-1-5: 7-(5-Carboxypentyl)amino-3-(5-chloro-benzoxazol-2-yl)coumarin

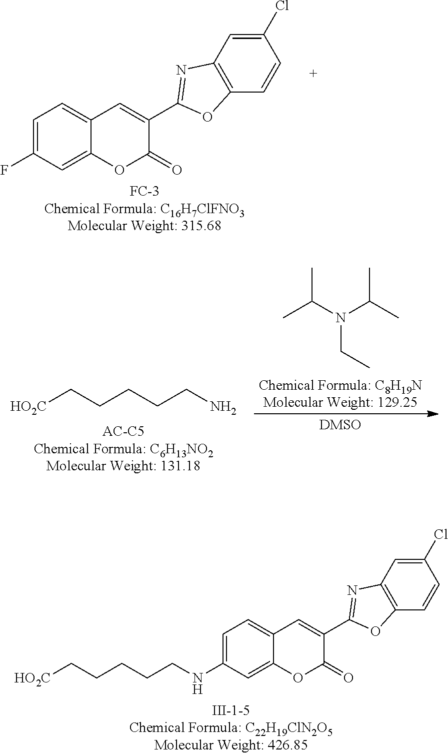

3-(5-Chloro-benzoxazol-2-yl)-7-fluoro-coumarin (FC-3, 0.32 g, 1 mmol, 1 eqv) and 6-aminohexanoic acid (AC-C5, 0.26 g, 2 mmol, 2 eqv) were added to anhydrous dimethyl sulfoxide (DMSO, 5 mL) in round bottomed flask. After the addition was complete, the mixture was stirred for a few minutes at room temperature and then DIPEA (0.52 g, 4 mmol, 2 eqv) was added. The reaction mixture was stirred for 7 hours at temperature 135° C. Additional portions of 6-aminohexanoic acid (AC-1, 0.13 g, 1 mmol, 1 eqv) and DIPEA (0.26 g, 2 mmol, 2 eqv) were added and heating was continued at 135° C. for 5 hours. After standing at room temperature for 1 hour, the pale-yellow reaction mixture was diluted with water (15 mL) and was stirred overnight. The resulting precipitate was collected by suction filtration. Yield 0.09 g (21%). Purity, structure and composition of the product were confirmed by HPLC, NMR and LCMS. MS (DUIS): MW Calculated 426.10. Found m/z: (+) 427 (M+1)⁺; (−) 425 (M−1), 851 (2M−1).

### Example 38: Compound III-2A 7-(5-Carboxypentyl)amino-3-(benzothiazol-2-yl)coumarin-6-sulfonic acid and Compound III-2B 7-(5-Carboxypentyl)amino-3-(benzothiazol-2-yl)coumarin-8-sulfonic acid

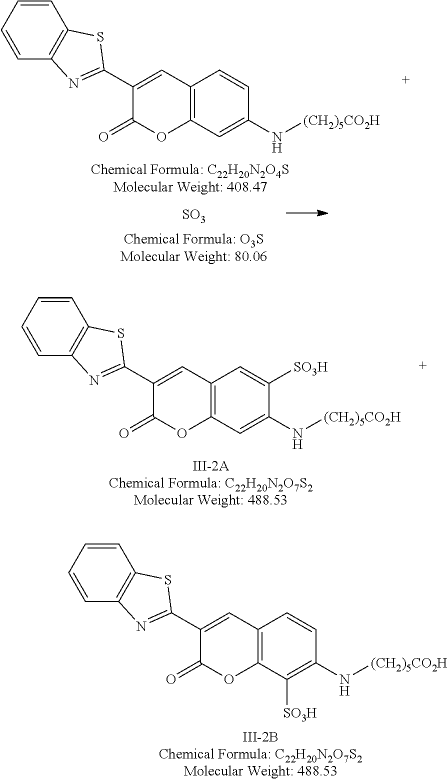

Compound III-1-1 (0.1 g, 0.245 mmol) was added in small portions with stirring to 20% fuming sulfuric acid (1 mL) that was cooled in a dry-ice/acetone bath. After the addition was complete, the mixture was stirred for 1 hour at 0° C., warmed to room temperature, and then stirred for 2 hours at room temperature. The solution was poured into anhydrous ether (25 mL). After standing at room temperature for 1 hour, the resulting precipitate was collected by suction filtration. Yield 78 mg (65%). ¹H NMR (d₆-DMSO) showed compound 2A plus a small amount (˜ 4%) of compound 2B.

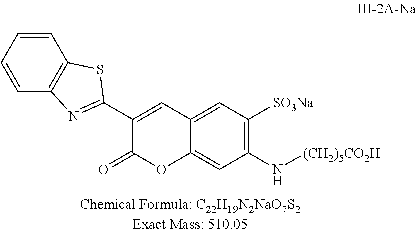

Example compound III-2A, Sodium Salt: The precipitate from above was resuspended in water (2 mL) and the pH of the suspension was adjusted to ˜ 5 by addition of 5 M NaOH solution. The resulting mixture was poured into 10 mL of methanol and the suspension was filtered. The filtrate was evaporated to dryness to give the dye as sodium salt (III-2A-Na). Purity, structure and composition were confirmed by HPLC, NMR and LCMS. MS (DUIS): MW Calculated 488.07. Found m/z: (+) 489 (M+1)⁺; (−) 243 (M−1)²⁻, 487 (M−1)⁻.

Preparation of Triethylammonium Salts of compounds III-2A and III-2B: Compound III-1-1 (0.41 g, 1 mmol) was added in small portions with stirring to 20% fuming sulfuric acid (5 mL) that was cooled in a dry-ice/acetone bath. After the addition was complete, the mixture was stirred for 1 hour at 0° C., warmed to room temperature, and then stirred for 2 hours at room temperature. The solution was poured into anhydrous ether (50 mL). After standing at room temperature for 1 hour, the organic solvent layer is decanted and the semi-solid bottom layer was dissolved in acetonitrile-water (1:1, 10 mL). The pH of the solution was adjusted to ˜ 7.0 by addition of 2 M TEAB solution in water. The resulting solution was filtered through a 20 m Nylon filter and the isomers were separated by preparative HPLC. The solution of the isomers were concentrated in vacuo then re-dissolved in water (20 μL) and solvent removed in vacuo to dryness to give the dyes as triethylammonium salts. Purity and composition were confirmed by HPLC and LCMS.

### Example 39: Compound III-3 7-(5-Carboxypentyl)amino-3-[5-sulfonato(benzothiazol-2-yl)-coumarin-6-sulfonate triethylammonium salt

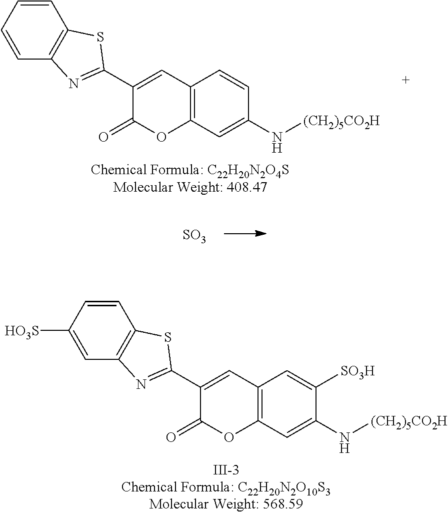

Compound III-1-1 (0.08 g, 0.2 mmol) was added in small portions with stirring to 20% fuming sulfuric acid (2 mL) that was cooled in a dry-ice/acetone bath. After the addition was complete, the mixture was stirred for 1 hour at 0° C., warmed to room temperature, and then stirred for 2 hours at 70° C. The mixture was then stirred overnight at room temperature. The solution was poured into anhydrous ether (30 mL). After stirring at room temperature for 1 hour, the resulting precipitate was collected by suction filtration. Yield 43 mg (38%).

The precipitate was resuspended in water (2 mL) and the pH of the suspension was adjusted to ˜ 7.5 by addition of 2 M TEAB solution in water. The resulting mixture was filtered through a 20 m Nylon filter and purified by preparative HPLC. The dye fraction was concentrated in vacuo then re-dissolved in water (20 μL) and solvent removed in vacuo to dryness to give the dye as the bis-triethylammonium salt. Purity and composition were confirmed by HPLC and LCMS. MS (DUIS): MW Calculated 568.03. Found m/z: (+) 569 (M+1)⁺.

Fluorescence intensities of dye solutions were compared with a commercial dye for the same spectral region. The results are shown in Table 3 and demonstrate significant advantages of the dyes for fluorescence based analytical applications.

TABLE 3
Spectral properties of the fluorescent dyes disclosed in the examples.

| | | Spectral properties | | |
|---|---|---|---|---|
| | | in EtOH-Water 1:1 | | |
| | | | | Relative Fluor.* |
| | | | Fluorescence | Intensity, |
| Number | Structure | Abs. max nm | max nm | % |
| III-1-1 | 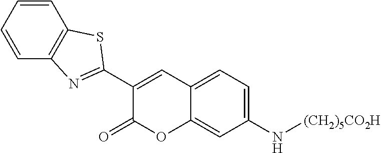 | 460 | 499 | 275 |
| III-1-2 | 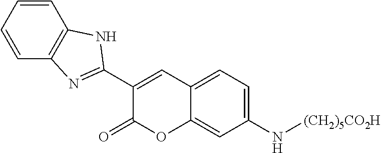 | 437 | 488 | 175 |
| III-1-3 | 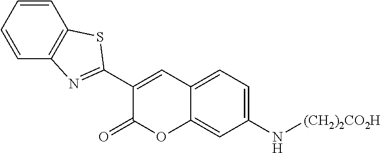 | 453 | 499 | 230 |
| III-1-4 | 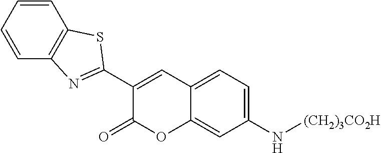 | 455 | 500 | 220 |
| III-1-5 | 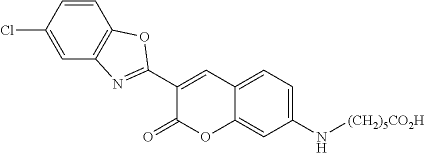 | 430 | 490 | 200 |
| III-2A | 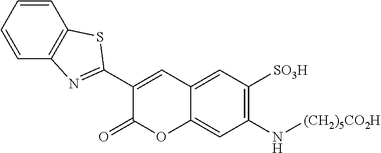 | 465 | 503 | 395 |
| III-2B | 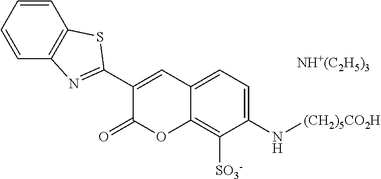 | 466 | 505 | 280 |
| III-3 | | 472 | 515 | 330 |
| Standard | Atto465 from AttoTec | 455 | 508 | 100 |

*Excitation of fluorescence @ 460 nm

### Example 40: General Procedure for the Synthesis of Fully Functional Nucleotide Conjugates with Fluorescent Dyes

Coumarin fluorescent dyes disclosed herein were coupled with appropriate amino-substituted adenine (A) and cytosine (C) nucleotide derivatives A-LN3-NH₂ or C-LN3—NH₂:

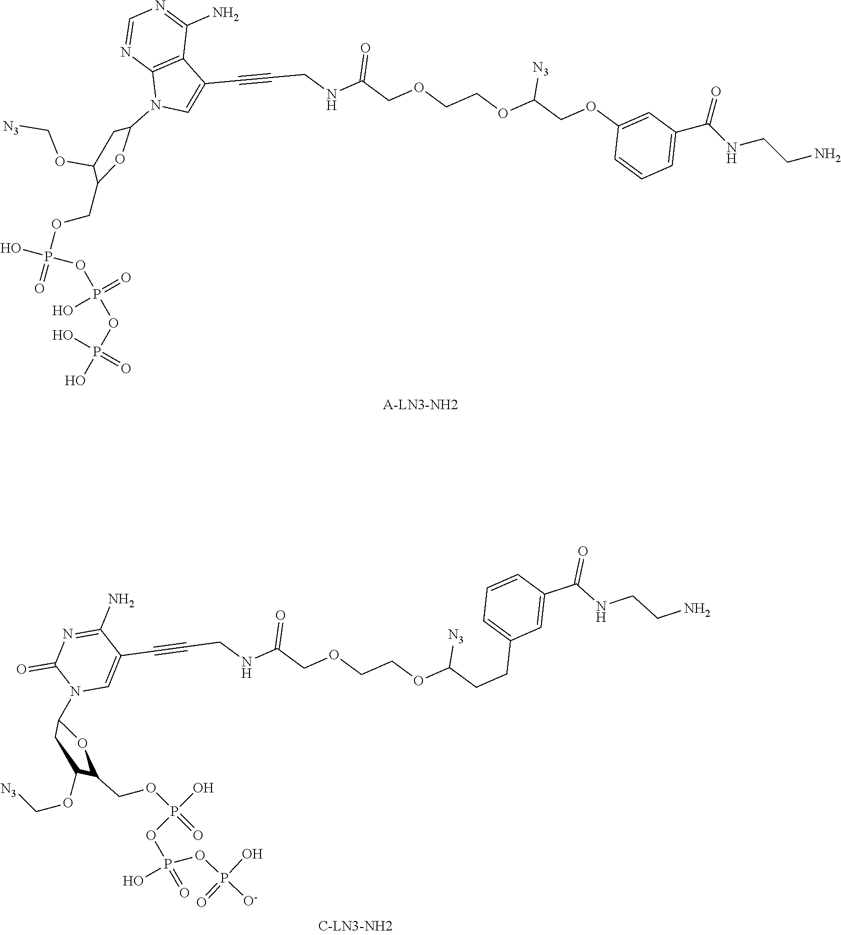

after activation of carboxylic group of a dye with appropriate reagents according to the following adenine scheme:

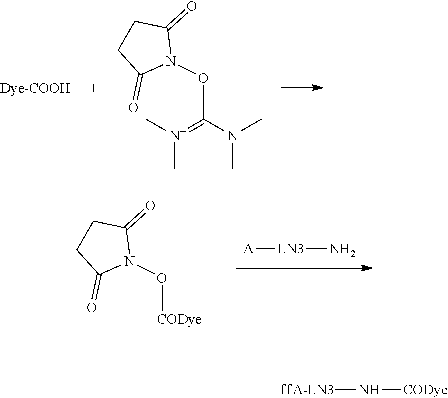

The general product for the adenine coupling is as shown below:

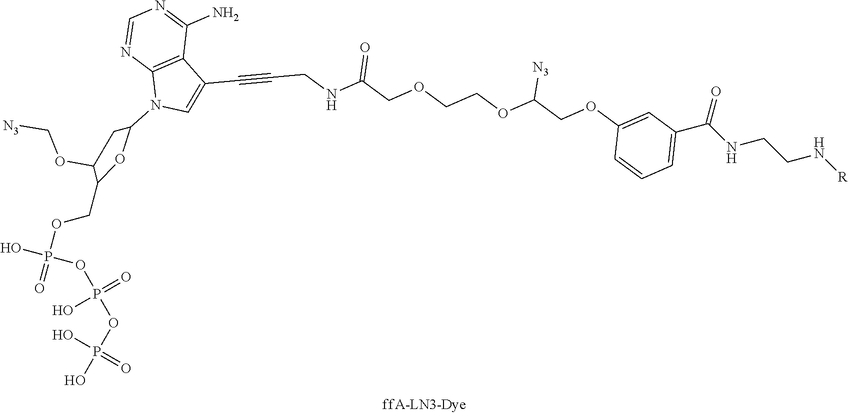

ffA-LN3-Dye refers to a fully functionalized A nucleotide with an LN3 linker and labeled with a coumarin dye disclosed herein. The R group in each of the structures refers to the coumarin dye moiety after conjugation.

The dye (10 μmol) is dried by placing into a 5 mL round-bottomed flask and is dissolved in anhydrous dimethylformamide (DMF, 1 mL) then the solvent is distilled off in vacuo. This procedure is repeated twice. The dried dye is dissolved in anhydrous N,N-dimethylacetamide (DMA, 0.2 mL) at room temperature. N,N,N′,N′-Tetramethyl-O—(N-succinimidyl)uronium tetrafluoroborate (TSTU, 1.5 eq., 15 μmol, 4.5 mg) is added to the dye solution, then DIPEA (3 eq., 30 μmol, 3.8 mg, 5.2 μL) is added via micropipette to this solution. The reaction flask is sealed under nitrogen gas. The reaction progress is monitored by TLC (eluent Acetonitrile-Water 1:9) and HPLC. Meanwhile, a solution of the appropriate amino-substituted nucleotide derivative (A-LN3-NH₂, 20 mM, 1.5 eq, 15 μmol, 0.75 mL) is concentrated in vacuo then re-dissolved in water (20 μL). A solution of the activated dye in DMA is transferred to the flask containing the solution of N-LN3-NH₂. More DIPEA (3 eq, 30 μmol, 3.8 mg, 5.2 μL) is added along with triethylamine (1 μL). Progress of coupling is monitored hourly by TLC, HPLC, and LCMS. When the reaction is complete, triethylamine bicarbonate buffer (TEAB, 0.05 M˜ 3 mL) is added to the reaction mixture via pipette. Initial purification of the fully functionalized nucleotide is carried out by running the quenched reaction mixture through a DEAE-Sephadex® column to remove most of remaining unreacted dye. For example, Sephadex is poured into an empty 25 g Biotage cartridge, solvent system TEAB/MeCN. The solution from the Sephadex column is concentrated in vacuo. The remaining material is re-dissolved in the minimum volume of water and acetonitrile, before filtering through a 20 m Nylon filter. The filtered solution is purified by preparative-HPLC. The composition of prepared compounds was confirmed by LCMS.

TABLE 4
Structure and spectral properties of various nucleotides labeled
with coumarin based dyes disclosed herein.

| | Spectral properties in SRE | | |
|---|---|---|---|
| | Absorption, | Fluorescence, | Relative Fluor. |
| Compd. | nm | nm | Intensity, % |
| ffA-III-1-1 | 448 | 505 | 480 |
| ffA-III-1-3 | 454 | 499 | 500 |
| ffA-III-2A | 475 | 510 | 575 |
| ffA-Standard | 465 | 504 | 100 |

A comparison of fluorescence intensities in solution of nucleotides labeled with dyes disclosed herein with appropriate data for nucleotides labeled with a commercial dye for the same spectral region (Atto465 from AttoTec GmbH) demonstrate the advantage of the dyes described herein for labeling of biomolecules to use in fluorescence based analytical applications.

A. Example Red and Green Dyes

Some aspects of the disclosure provide for compounds of the formula (V) or mesomeric forms thereof:

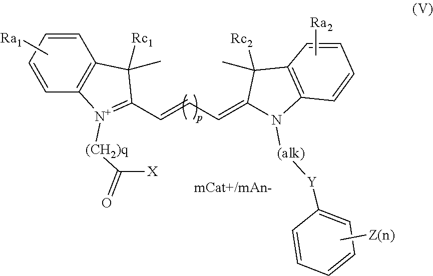

wherein mCat+ or mAn− is an organic or inorganic positively/negatively charged counterion and
m is an integer 0-3;
p is an integer 1-2;
q is an integer 1-5;
alk is a chain of 1-5 carbon atoms optionally containing one or more double or triple bonds;

### Y is S, O or CH₂;

### Z is OH;

n is an integer 0-3;
X is OH or O⁻ or an amide or ester conjugate thereof;
each of Ra₁ and Ra₂ is independently H, SO₃⁻, sulfonamide, halogen, or a further ring fused to an adjacent carbon atom; and
each of Rc₁ and Rc₂ is independently alkyl or substituted alkyl.

In some aspects, each of Rc₁ and Rc₂ is independently alkyl or substituted alkyl, wherein at least one of Ra₁ or Ra₂ is SO₃⁻, or Ra₁ or Ra₂ is a further ring fused to an adjacent carbon atom, the further ring having an SO₃⁻, or Rc₁ or Rc₂ is an alkyl sulfonic acid group. In some aspects, each of Rc₁ and Rc₂ is independently alkyl or substituted alkyl, wherein when n is 0, Y is S or O. In some aspects, each of Rc₁ and Rc₂ is independently alkyl or substituted alkyl, wherein at least one of Ra₁ or Ra₂ is SO₃⁻, or Ra₁ or Ra₂ is a further ring fused to an adjacent carbon atom, the further ring having an SO₃—, or Rc₁ or Rc₂ is an alkyl sulfonic acid group and wherein when n is 0, Y is S or O.

The molecules may contain one or more sulphonamide or SO₃— moieties at position Ra. Ra₁ and/or Ra₂ may be SO₃⁻ or sulphonamide. The other Ra (Ra₁ or Ra₂) can be independently H, SO₃⁻, sulphonamide, halogen, or a further ring fused to an adjacent carbon atom. Ra₁ or Ra₂ can be H. Ra₁ or Ra₂ can be SO₃⁻. Ra₁ can be different to Ra₂, for example the structure can have a single sulfonamide group at Ra₁, and H as Ra₂. Ra₁ and Ra₂ can both be sulphonamide. The sulphonamide can be SO₂NH₂ or SO₂NHR, where R is an alkyl, substituted alkyl, aryl or substituted aryl group. Where neither Ra₁ or Ra₂ is a SO₃ or a further ring fused to an adjacent carbon atom, then Rc₁ or Rc₂ must be an alkyl sulfonic acid group.

Ra₁ or Ra₂ can be a further aliphatic, aromatic or heterocyclic ring fused to adjacent carbons of the indole ring. For example, in such cases when an aromatic ring is fused the dyes end group can represent a structure of type:

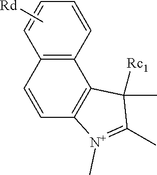

where Rd can be H, alkyl, substituted alkyl, aryl, substituted aryl, halogen, carboxy, sulphonamide, or sulfonic acid.

Thus, some dyes of the disclosure can be described by Formula (VC) or (VD) or mesomeric forms thereof:

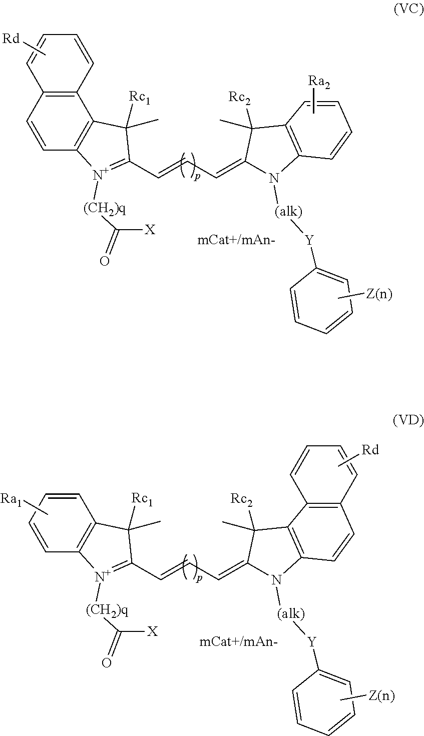

wherein mCat+ or mAn− is an organic or inorganic positively/negatively charged counterion and
m is an integer 0-3;
p is an integer 1-2;
q is an integer 1-5;
alk is a chain of 1-5 carbon atoms optionally containing one or more double or triple bonds;

### Y is, O or CH₂;

### Z is OH;

n is an integer 0-3;
X is OH or O⁻ or an amide or ester conjugate thereof;
each of Ra₁ and Ra₂ is independently H, SO₃—, sulfonamide, halogen, or a further ring fused to an adjacent carbon atom;
each of Rc₁ and Rc₂ is independently alkyl or substituted alkyl; and
Rd is H, alkyl, substituted alkyl, aryl, substituted aryl, halogen, carboxy, sulphonamide, or sulfonic acid.

In some aspects, Rd is H, alkyl, substituted alkyl, aryl, substituted aryl, halogen, carboxy, sulphonamide, or sulfonic acid, wherein at least one of Ra₁ or Ra₂ is SO₃⁻, or Rd is SO₃⁻, or Rc₁ or Rc₂ is an alkyl sulfonic acid group. In some aspects, Rd is H, alkyl, substituted alkyl, aryl, substituted aryl, halogen, carboxy, sulphonamide, or sulfonic acid, wherein when n is 0, Y is S or O. In some aspects, Rd is H, alkyl, substituted alkyl, aryl, substituted aryl, halogen, carboxy, sulphonamide, or sulfonic acid, wherein at least one of Ra₁ or Ra₂ is SO₃, or Rd is SO₃—, or Rc₁ or Rc₂ is an alkyl sulfonic acid group and wherein when n is 0, Y is S or O.

In formula (VC) or (VD) the additional rings fused to adjacent carbon atoms of the indole ring may be optionally substituted, for example with sulfonic acid or sulphonamide.

The C(═O)—X carboxy group or its derivatives is attached to the indole nitrogen atom by an alkyl chain of length q, where q is 1-5 carbon or hetero-atoms. The chain may be (CH₂)_{q} where q is 1-5. The group may be (CH₂)₅COOH.

The molecules can contain one or more alkyl-sulfonate moieties at position Rc. Either Rc₁ and/or Rc₂ may be alkyl-SO₃—. The other Rc (Rc₁ or Rc₂) can be independently alkyl or substituted alkyl. Rc₁ and Rc₂ may be independently methyl, ethyl, propyl, butyl, pentyl, hexyl or (CH₂)ₜSO₃H, where t is 1-6. t may be 1-4. t may be 4. Rc₁ and Rc₂ may be a substituted alkyl group. Rc₁ and Rc₂ may contain a COOH or —SO₃H moiety or their ester or amide derivatives.

In certain implementations, when one of Ra₁ or Ra₂ is SO₃⁻, and the other of Ra₁ or Ra₂ is H or SO₃⁻, either Rc₁ or Rc₂ can also be an alkyl sulfonic acid group.

The COOH group shown as C(═O)—X can act as a linking moiety for further attachment or is linked to a further molecule. Once conjugation has occurred, the COOH or COO— is converted into an amide or ester.

Examples of compounds include structures according to formula (VI) or (VIa) or mesomeric forms thereof:

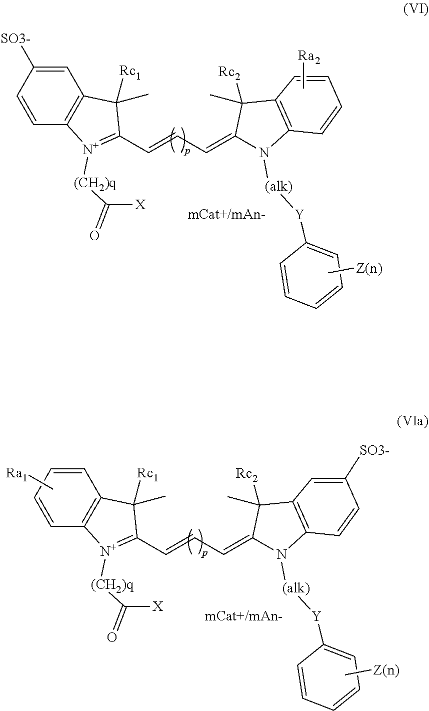

wherein mCat+ or mAn− is an organic or inorganic positively/negatively charged counterion and
m is an integer 0-3;
p is an integer 1-2;
q is an integer 1-5;
alk is a chain of 1-5 carbon atoms optionally containing one or more double or triple bonds;

### Y is S, O or CH₂;

### Z is OH;

n is an integer 0-3;
X is OH or O⁻ or an amide or ester conjugate thereof;
each of Ra₁ and Ra₂ is independently H, SO₃⁻, sulfonamide, halogen, or a further ring fused to an adjacent carbon atom; and
each of Rc₁ and Rc₂ is independently alkyl or substituted alkyl.

In some aspects, each of Rc₁ and Rc₂ is independently alkyl or substituted alkyl, wherein when n is 0, Y is S or O.

Further examples of compounds include structures according to formula (VIIa) or (VIIb):

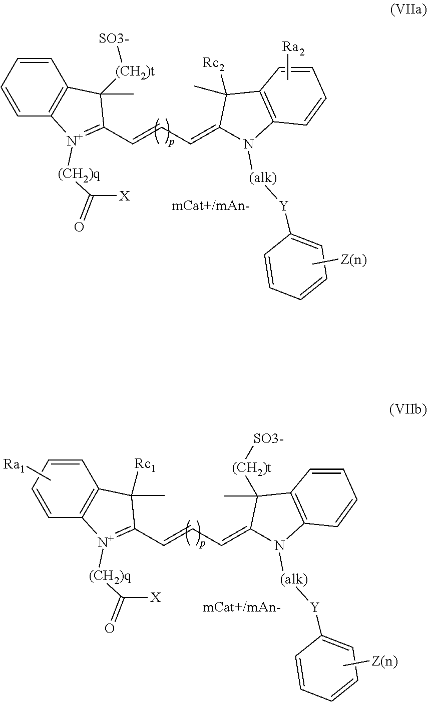

wherein mCat+ or mAn− is an organic or inorganic positively/negatively charged counterion and
m is an integer 0-3;
p is an integer 1-2;
q is an integer 1-5;
alk is a chain of 1-5 carbon atoms optionally containing one or more double or triple bonds;
t is an integer 1-6;

### Y is S, O or CH₂;

### Z is OH;

n is an integer 0-3;
X is OH or O⁻ or an amide or ester conjugate thereof;
each of Ra₁ and Ra₂ is independently H, SO₃⁻, sulfonamide, halogen, or a further ring fused to an adjacent carbon atom; and
each of Rc₁ and Rc₂ is independently alkyl or substituted alkyl.

In some aspects, each of Rc₁ and Rc₂ is independently alkyl or substituted alkyl, wherein when n is 0, Y is S or O.

Further examples of compounds include structures according to formula (VIIIa) to (VIIId):

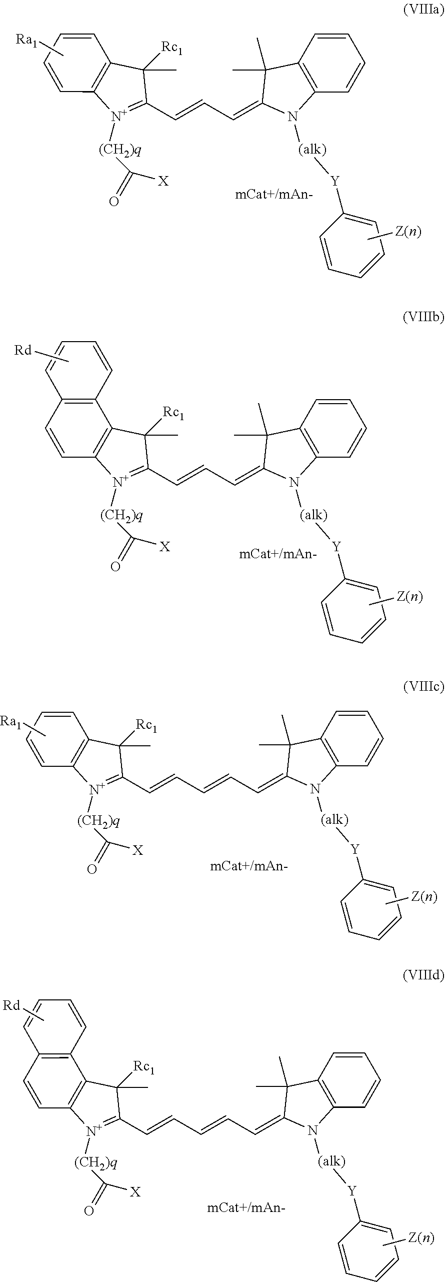

wherein mCat+ or mAn− is an organic or inorganic positively/negatively charged counterion and
m is an integer 0-3;
q is an integer 1-5;
alk is a chain of 1-5 carbon atoms optionally containing one or more double or triple bonds;

### Y is S, O or CH₂;

### Z is OH;

n is an integer 0-3;
X is OH or O⁻ or an amide or ester conjugate thereof;
Ra₁ is H, SO₃⁻, sulfonamide, halogen, or a further ring fused to an adjacent carbon atom;
Rc₁ is alkyl or substituted alkyl; and
Rd is H, alkyl, substituted alkyl, aryl, substituted aryl, halogen, carboxy, sulphonamide, or sulfonic acid.

Further examples of compounds include structures according to formula (IXa) to (IXd):

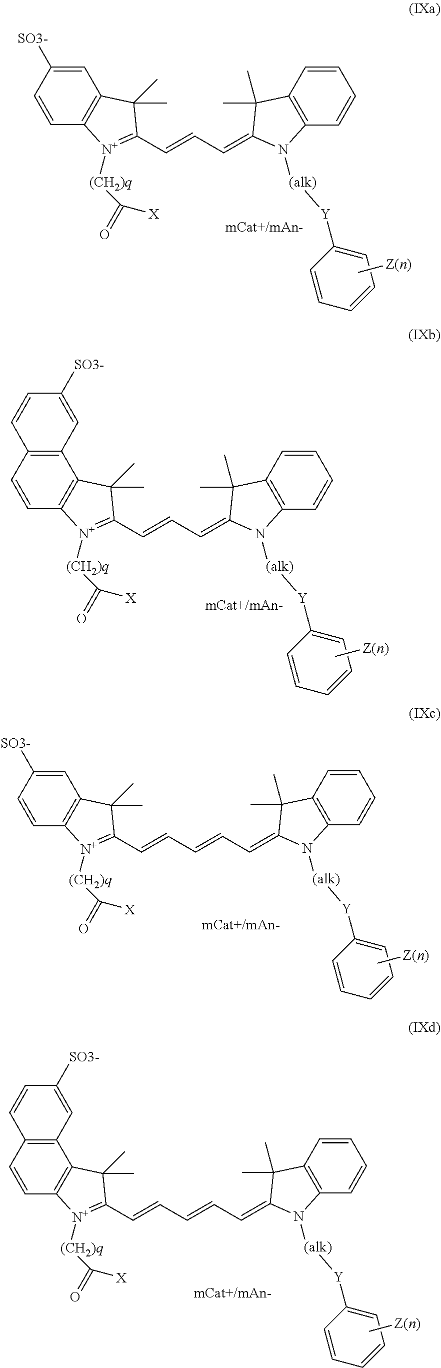

wherein mCat+ or mAn− is an organic or inorganic positively/negatively charged counterion and
m is an integer 0-3;
q is an integer 1-5;
alk is a chain of 1-5 carbon atoms optionally containing one or more double or triple bonds;

### Y is S, O or CH₂;

### Z is OH;

n is an integer 0-3; and
X is OH or O⁻ or an amide or ester conjugate thereof.

Further examples of compounds include structures according to formula (Xa) to (Xd):

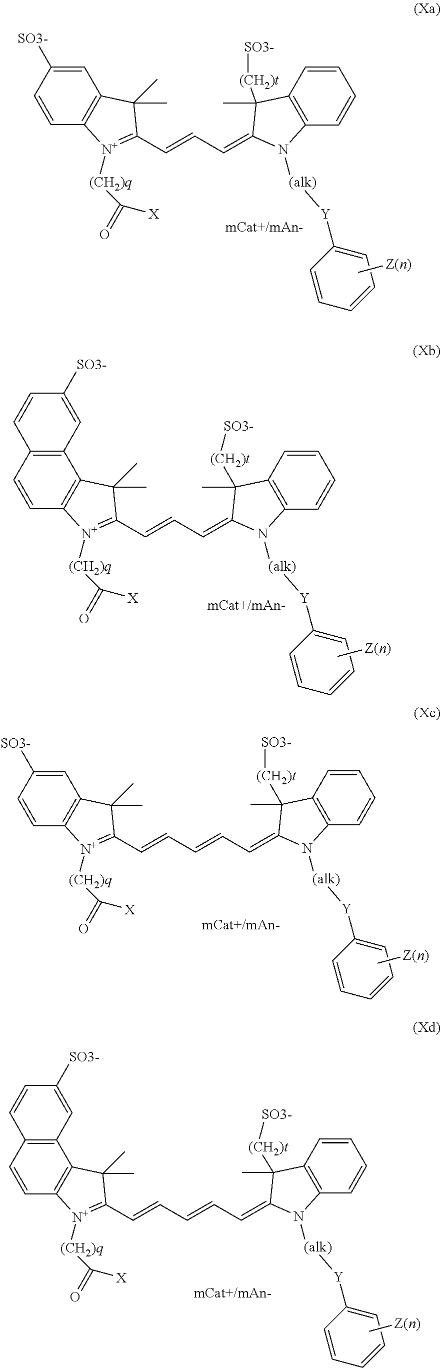

wherein mCat+ or mAn− is an organic or inorganic positively/negatively charged counterion and
m is an integer 0-3;
q is an integer 1-5;
alk is a chain of 1-5 carbon atoms optionally containing one or more double or triple bonds;
t is an integer 1-6;

### Y is S, O or CH₂;

### Z is OH;

n is an integer 0-3; and
X is OH or O⁻ or an amide or ester conjugate thereof.

In the foregoing implementations, alk is an alkyl, alkenyl or alkynyl chain of 1-carbon atoms optionally containing one or more double or triple bonds. Alk can be a group (CH₂)r where r is 1-5. Alk can be (CH₂)₃. Alternatively the carbon chain may contain one or more double bonds or triple bonds. The chain may contain a linkage —CH₂—CH═CH—CH₂—, optionally with further CH₂ groups. The chain may contain a linkage —CH₂—C≡C—CH₂—, optionally with further CH₂ groups.

In any of the examples given in formula V to XII; q can equal 5. In any of the examples given in formula VII, formula X or formula XI; t can equal 4. In any of the examples given in formula V to X; n can equal 1-3. In any of the examples given in formula V to X; n can equal 1. In any of the examples given in formula V to X; n can be an integer 0-1. Where n is 1, the OH group can be at any position on the ring. The OH group can be at the 4 position. Where n is 2 or 3, the OH groups can be at any positions on the phenyl ring. In any of the examples given in formula V to X; when n is zero, Y can equal O or S and not CH₂. In any of the examples given in formula V to X; Y can equal O. In any of the examples given in formula V to X; Y can equal O. Where Y is O, n can be 0-3. Where Y is CH₂, n can be 1-3.

Further examples of compounds include structures according to formula (XIa) to (XId):

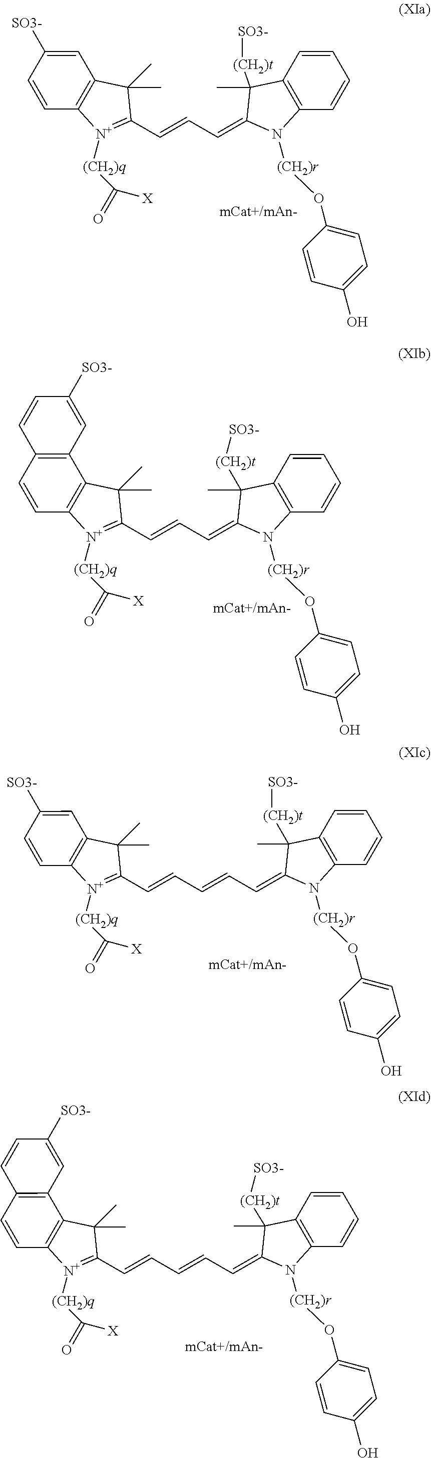

wherein mCat+ or mAn− is an organic or inorganic positively/negatively charged counterion and
m is an integer 0-3;
q is an integer 1-5;
r is an integer 1-5;
t is an integer 1-6; and
X is OH or O⁻ or an amide or ester conjugate thereof.

Further examples of compounds include structures according to formula (XIIa) to (XIId):

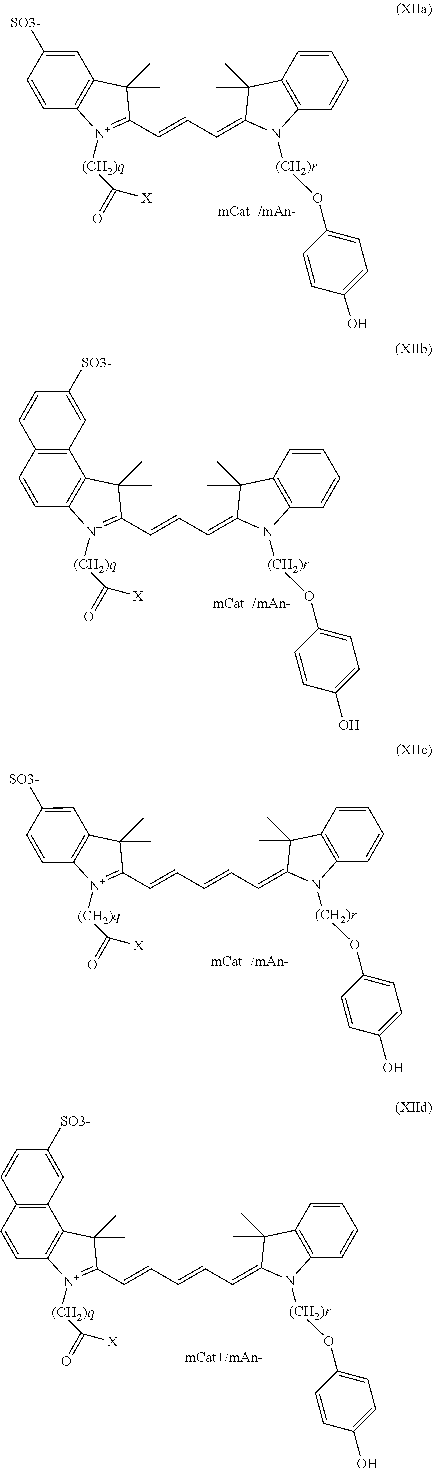

wherein mCat+ or mAn− is an organic or inorganic positively/negatively charged counterion and
m is an integer 0-3;
q is an integer 1-5;
r is an integer 1-5; and
X is OH or O⁻ or an amide or ester conjugate thereof.

In any of the examples given in formula XI to XII; r can equal 3.

A particularly useful compound is a nucleotide or oligonucleotide labeled with a dye as described herein. The labeled nucleotide or oligonucleotide may have the label attached to the nitrogen atom of indole via an alkyl-carboxy group to form an amide. The labelled nucleotide or oligonucleotide may have the label attached to the C5 position of a pyrimidine base or the C7 position of a 7-deaza purine base through a linker moiety.

The labeled nucleotide or oligonucleotide may also have a blocking group covalently attached to the ribose or deoxyribose sugar of the nucleotide. The blocking group may be attached at any position on the ribose or deoxyribose sugar. In particular implementations, the blocking group is at the 3′ OH position of the ribose or deoxyribose sugar of the nucleotide.

Provided herein are kits including two or more nucleotides wherein at least one nucleotide is a nucleotide labeled with a compound of the present disclosure. The kit may include two or more labeled nucleotides. The nucleotides may be labelled with two or more fluorescent labels. Two or more of the labels may be excited using a single excitation source, which may be a laser. For example, the excitation bands for the two or more labels may be at least partially overlapping such that excitation in the overlap region of the spectrum causes both labels to emit fluorescence. In particular implementations, the emission from the two or more labels will occur in different regions of the spectrum such that presence of at least one of the labels can be determined by optically distinguishing the emission.

The kit may contain four labeled nucleotides, where the first of four nucleotides is labeled with a compound as disclosed herein. In such a kit, the second, third, and fourth nucleotides can each be labeled with a compound that is optionally different from the label on the first nucleotide and optionally different from the labels on each other. Thus, one or more of the compounds can have a distinct absorbance maximum and/or emission maximum such that the compound(s) is(are) distinguishable from other compounds. For example, each compound can have a distinct absorbance maximum and/or emission maximum such that each of the compounds is distinguishable from the other three compounds. It will be understood that parts of the absorbance spectrum and/or emission spectrum other than the maxima can differ and these differences can be exploited to distinguish the compounds. The kit may be such that two or more of the compounds have a distinct absorbance maximum above 600 nm. The compounds can absorb light in the region above 640 nm. The kit may include any of the red, green, or blue wavelength light emitting compounds described herein.

The compounds, nucleotides or kits that are set forth herein may be used to detect, measure or identify a biological system (including, for example, processes or components thereof). Some techniques that can employ the compounds, nucleotides or kits include sequencing, expression analysis, hybridisation analysis, genetic analysis, RNA analysis, cellular assay (e.g. cell binding or cell function analysis), or protein assay (e.g. protein binding assay or protein activity assay). The use may be on an automated instrument for carrying out a particular technique, such as an automated sequencing instrument. The sequencing instrument may contain two lasers operating at different wavelengths.

Disclosed herein is a method of synthesising compounds of the disclosure. A compound of formula (XIII) and/or (XIII-1), (XIII-2) (XIII-3) or (XIII-4) or a salt thereof may be used as a starting material for the synthesis of symmetrical or unsymmetrical polymethine dyes:

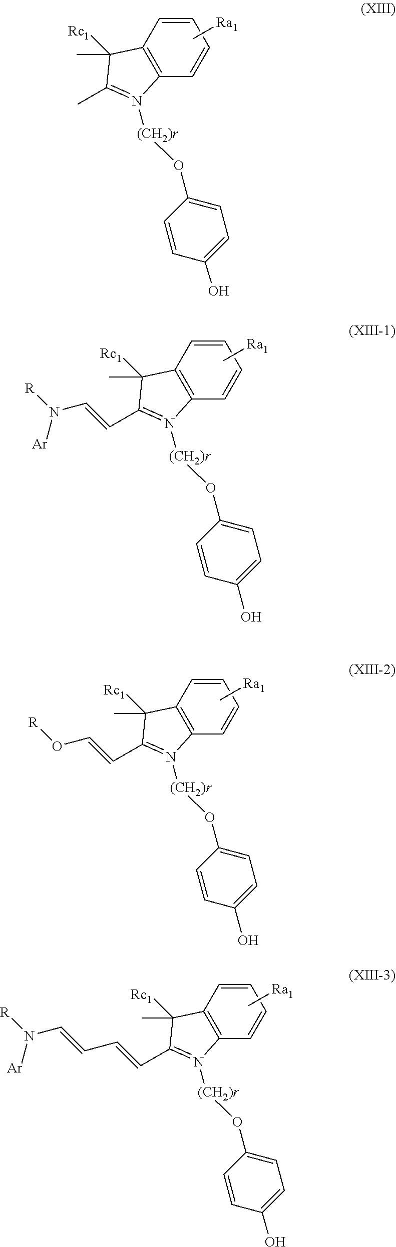

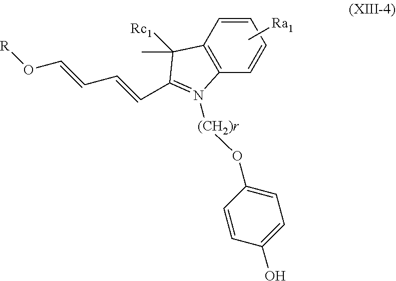

or a salt thereof wherein Ra₁ is H, SO₃⁻, sulfonamide, halogen, or a further ring fused to an adjacent carbon atom; Rc₁ is alkyl or substituted alkyl; Ar is an aromatic group and R is an alkyl group. Where specific examples of 4-hydroxyphenyl are shown, further hydroxyl groups may also be substituted on the ring in cases where n is greater than one. r can be equal to 3.

Disclosed herein is a method of synthesising compounds of the disclosure. A compound of formula (XIII-5) or a salt thereof may be used as a starting material for the synthesis of symmetrical or unsymmetrical polymethine dyes:

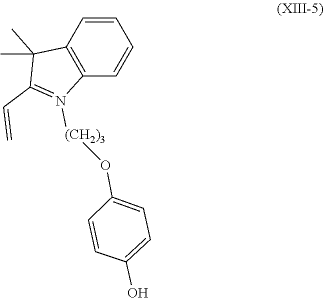

Further aspects of the disclosure provide polymethine dye compounds of the formula (XIV) or mesomeric forms thereof:

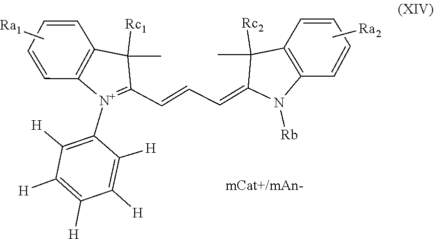

wherein mCat+ or mAn− is an organic or inorganic positively/negatively charged counterion and
m is an integer 0-3;
each of Ra₁ and Ra₂ is independently H, SO₃⁻, sulfonamide, halogen, or a further ring fused to an adjacent carbon atom;
Rb is optionally substituted aryl or optionally substituted alkyl;
each of Rc₁ and Rc₂ is independently alkyl or substituted alkyl; and
either Rb or one of Rc₁ or Rc₂ contains a linking moiety for further attachment or is linked to a further molecule.

Each Ra₁ or Ra₂ can be independently H, SO₃, sulphonamide, halogen, or a further ring fused to an adjacent carbon atom. Ra₁ or Ra₂ can be H. Ra₁ or Ra₂ can be SO₃⁻ Ra₁ can be different to Ra₂, for example the structure can have a single sulfonic acid group at Ra₁, and H as Ra₂. Ra₁ or Ra₂ can be sulphonamide. The sulphonamide can be SO₂NH₂ or SO₂NHR, where R is an alkyl, substituted alkyl, aryl or substituted aryl group.

Ra₁ or Ra₂ can be a further aliphatic, aromatic or heterocyclic ring fused to an adjacent carbon of the indole ring. For example, in such cases when an aromatic ring is fused the dyes end group can represent a structure of type:

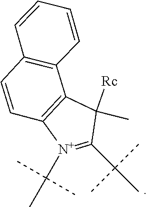

Thus the dyes of the disclosure can be described by Formula (XIVA), (XIVB) or (XIVC):

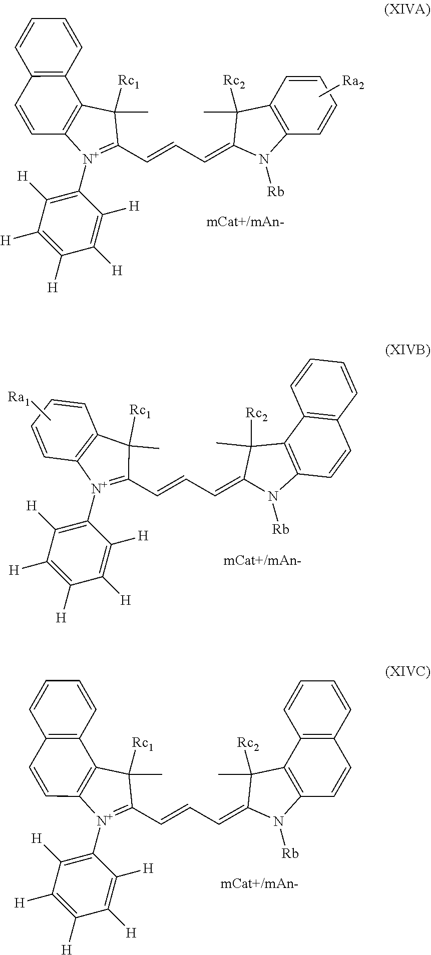

In formula (XIVA), (XIVB) and (XIVC) one or both additional rings fused to an adjacent carbon atoms of the indole ring may be optionally substituted, for example with sulfonic acid or sulphonamide.

The compound may be where one of the Ra groups is a further fused ring forming a structure of formula (XV):

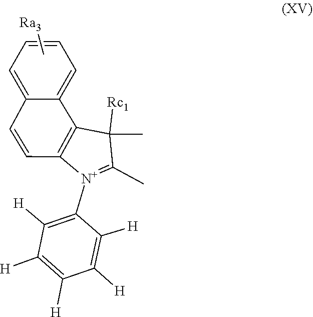

wherein Ra₃ is H, SO₃⁻, sulphonamide or halogen; and
Rc₁ is alkyl or substituted alkyl.

Rb can be optionally substituted aryl or optionally substituted alkyl. Rb can be alkyl. Rb can be methyl, ethyl, propyl, butyl, pentyl or hexyl. The alkyl chain can be further substituted, for example with carboxy or sulfonic groups. The Rb can be used for further conjugation. For example if Rb contains a COOH moiety, this can be conjugated with further molecules in order to attach the label. In the case of biomolecule, protein, DNA labelling and suchlike, the conjugation can be carried out via Rb. Rb can form amide or ester derivatives once the conjugation has occurred. The compound may be attached to a nucleotide or oligonucleotide via Rb.

Rb can be aryl or substituted aryl. Rb can be phenyl.

Each Rc₁ and Rc₂ can be independently alkyl or substituted alkyl. Rc₁ and Rc₂ may be methyl, ethyl, propyl, butyl, pentyl, hexyl or (CH₂)_{q}SO₃H, where q is 1-6. q may be 1-3. Rc₁ and Rc₂ may be a substituted alkyl group. Rc₁ and Rc₂ may contain a COOH or —SO₃H moiety or their ester or amide derivatives.

Either Rb or Rc₁ or Rc₂ contains a linking moiety for further attachment or is linked to a further molecule. Rb or Rc₁ or Rc₂ may contain a carboxy or carboxylate (COOH or COO) moiety. Once conjugated has occurred, Rb or Rc₁ or Rc₂ may contain an amide or ester.

Examples of compounds include:

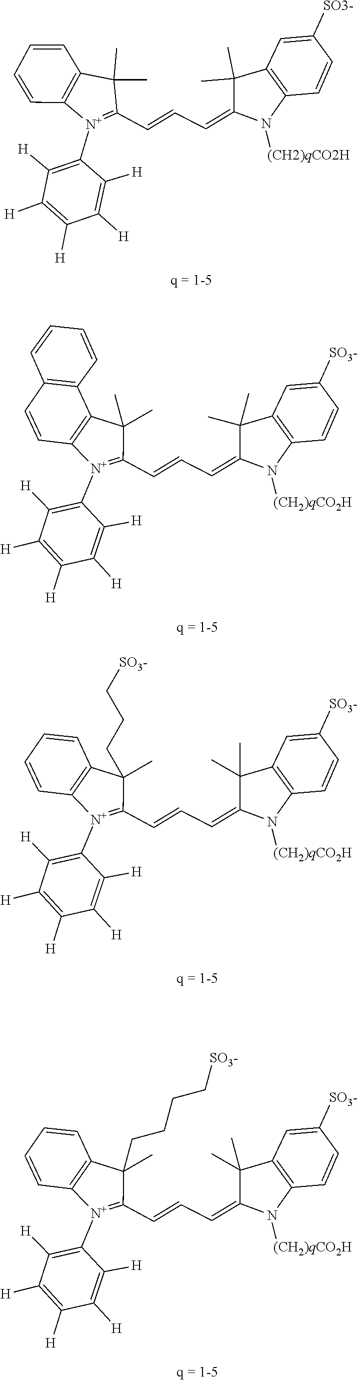

or salts thereof.

Disclosed herein is a method of synthesising compounds of the disclosure. A compound of formula (XVI) and/or (XVII), (XVI2) or a salt thereof may be used as a starting material for the synthesis of symmetrical or unsymmetrical polymethine dyes:

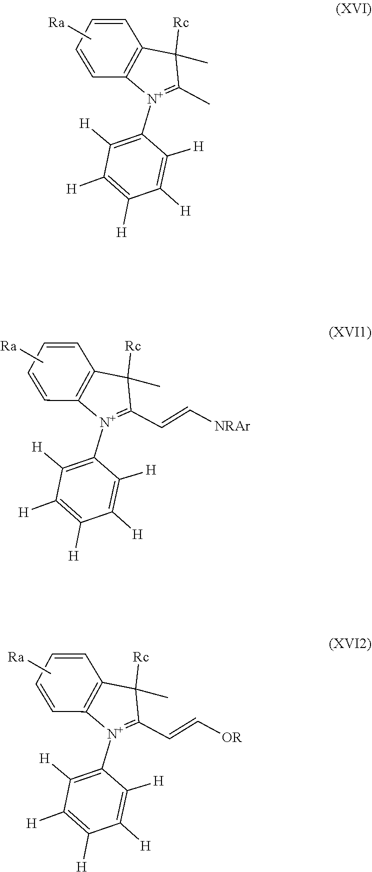

wherein Ra is H, SO₃⁻, sulphonamide, halogen, or a further ring fused to an adjacent carbon atoms;
Rb is optionally substituted aryl or optionally substituted alkyl; and
Rc is alkyl or substituted alkyl.

Particular excitation wavelengths can be 532 nm, 630 nm to 700 nm, particularly 660 nm.

### Example 41: Compound XVII 2,3,3-Trimethyl-1-phenyl-3H-indolium-5-sulfonate

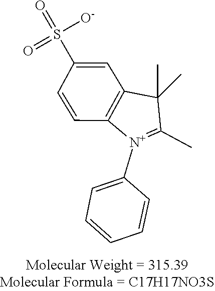 [SMILES: CC1=[N+](c2ccccc2)c2ccc(S(=O)(=O)[O-])cc2C1(C)C]

2-Methylene-3,3-trimethyl-1-phenyl-2,3-dihydro-1H-indole (1 g, 4.25 mmol) was dissolved in 1 ml of sulphuric acid at temperature <5° C. and 1 ml fuming sulphuric acid (20%) was added with stirring. The solution was stirred at room temperature 1 h then heated at 60° C. for 3 h. Product precipitated with diethyl ether washed with acetone and ethanol. Yield 0.7 g (52%). The structure was confirmed by NMR.

### Example 42: Compound XVIII 2-(2-Anilinovinyl-1)-3,3-trimethyl-1-phenyl-3H-indolium-5-sulfonate

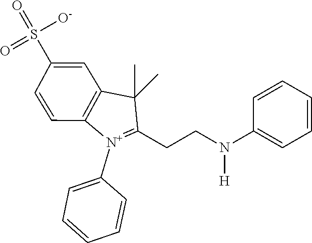 [SMILES: CC1(C)C(CCNc2ccccc2)=[N+](c2ccccc2)c2ccc(S(=O)(=O)[O-])cc21]

Reaction Scheme:

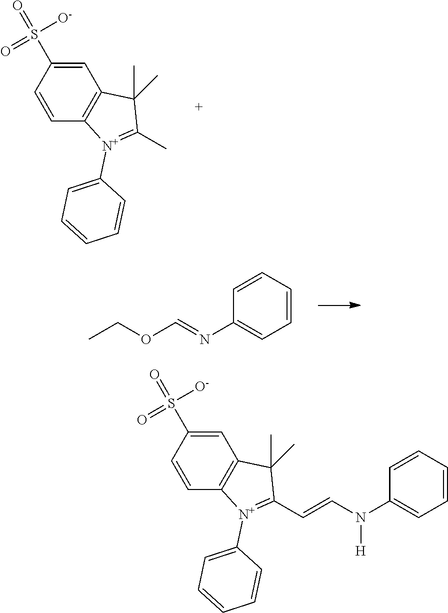

A mixture of 2,3,3-trimethyl-1-phenyl-3H-indolium-5-sulfonate (0.63 g) and ethyl N-phenylformimidate (0.5 g) was heated at 70° C. for 30 min. An orange melt formed. The product triturated with diethyl ether and filtered off. Yield 0.7 g (84%).

### Example 43: Compound XIX 2-(2-Acetanilidovinyl-1)-3,3-trimethyl-1-phenyl-3H-indolium-5-sulfonate

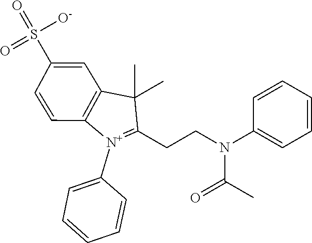 [SMILES: CC(=O)N(CCC1=[N+](c2ccccc2)c2ccc(S(=O)(=O)[O-])cc2C1(C)C)c1ccccc1]

Reaction Scheme:

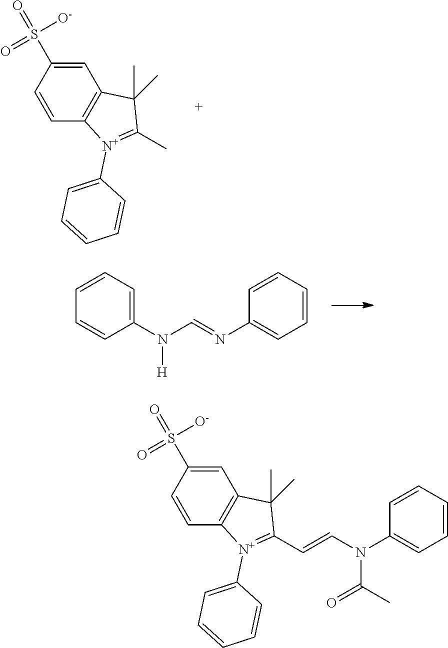

A mixture of 2,3,3-trimethyl-1-phenyl-3H-indolium-5-sulfonate (0.63 g), N,N′-diphenylformimidine (0.5 g), acetic acid (1 ml) and acetic anhydride (2 ml) was heated at 70° C. for 3 hours and then at 50° C. overnight. A yellow solution formed. The product was filtered off and washed with diethyl ether. Yield 0.69 g (75%).

### Example 44: Compound XX 1,2-dimethyl-1-(4-sulfonatobutyl)-3-phenyl-1H-benzo[e]indolium

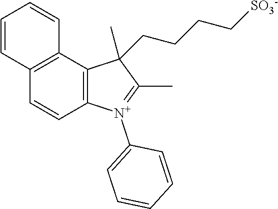

Reaction Scheme:

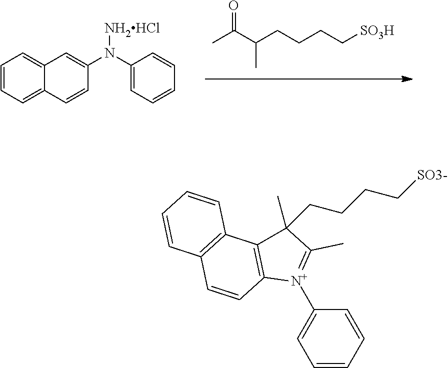

N-(2-Naphtyl),N-phenylhydrazine hydrochloride (19.51 mmol, 5.28 g), 5-methyl-6-oxoheptanesulfonic acid (17.18 mmol, 3.70 g) and anhydrous ZnCl₂ (17.18 mmol, 2.34 g) in absolute ethanol (30 ml) were stirred at room temperature for 30 min, then at 80° C. for 2 h. the reaction progress was checked by TLC (10% H₂O in CH₃CN). After completion the reaction was cooled down and the solvent removed under vacuum. The residue was dissolved in DCM and purified by flash column on silica-gel. Yield: 3.06 g, 42%.

Proton NMR: (MeOH-D4): 8.28 (0.5H, d, J=8 Hz); 8.05-8.02 (1H, m); 7.89 (0.5H, d, J=8 Hz); 7.75-7.66 (3H, m); 7.65-7.60 (1H, m); 1.49-1.43 (1.5H, m); 7.31-7.25 (2H, m); 7.16 (0.5H, d, J=9 Hz); 7.07 (0.5H, appt, J=7.4 Hz); 6.61 (0.5H, d, J=8 Hz); 2.85-2.35 (4H, m); 1.88 (3H, appd, J=9 Hz); 1.75-1.4 (5H, m); 1.35-1.25 (0.5H, m); 1.1-0.95 (0.5H, m); 0.8-0.65 (0.5H, m); 0.58-0.45 (0.5H, m).

### Example 45: Compound XXI 1,2-Dimethyl-1-(3-sulfonatopropyl)-3-phenyl-1H-benzo[e]indolium

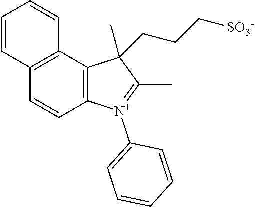

Reaction Scheme:

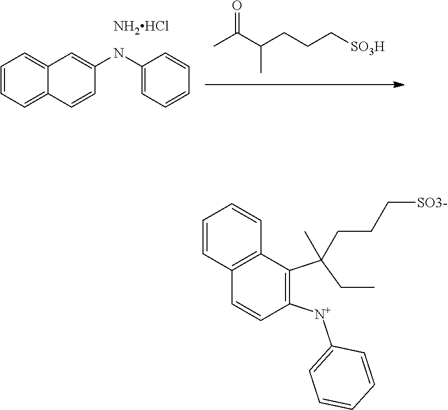

The title compound was prepared as the previous compound from N-(2-naphtyl)-N-phenylhydrazine hydrochloride and 4-methyl-5-oxopentanesulfonic acid. The product was purified by flash column on silicagel. Yield: 40%. Structure confirmed by NMR spectrum.

### Example 46: Compound XXII 2,3-Dimethyl-3-(4-sulfonatobutyl)-1-phenyl-3H-indolium

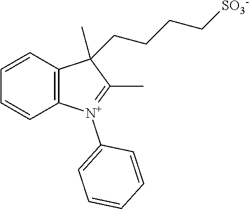

Reaction Scheme:

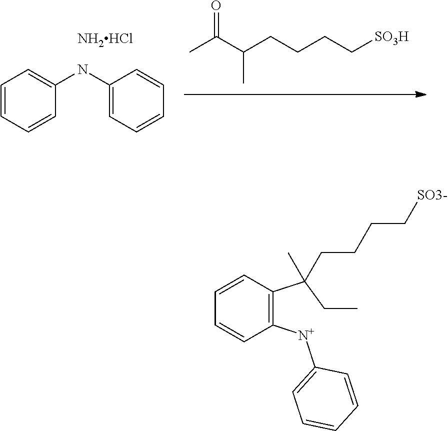

N,N-Diphenylhydrazine hydrochloride (0.01 mol, 2.2 g), 5-methyl-6-oxoheptanesulfonic acid (0.017 mol, 3.0 g) in glacial acetic acid (20 ml) were stirred at room temperature (˜20° C.) for an hour then at 100° C. for 3 hours (TLC check). The reaction mixture was cooled down and the solvent removed under vacuum. The residue was washed with diethyl ether and purified by flash column on silicagel. Yield: 2 g (56%). Structure confirmed by NMR spectrum.

### Example 47: Compound XXIII Indocarbocyanine

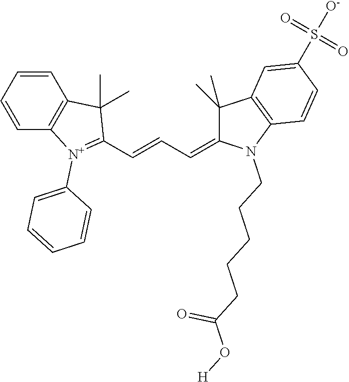 [SMILES: CC1(C)C(/C=C/C=C2/N(CCCCCC(=O)O)c3ccc(S(=O)(=O)[O-])cc3C2(C)C)=[N+](c2ccccc2)c2ccccc21]

Chemical Name: 2-{(5-[1-phenyl-3,3-dimethyl)-1,2-dihydro-3H-indol-2-ylidene]-1-propen-1-yl}-3,3-dimethyl-1-(5-carboxypenthyl)-indolium-5-sulfonate.

Reaction Scheme:

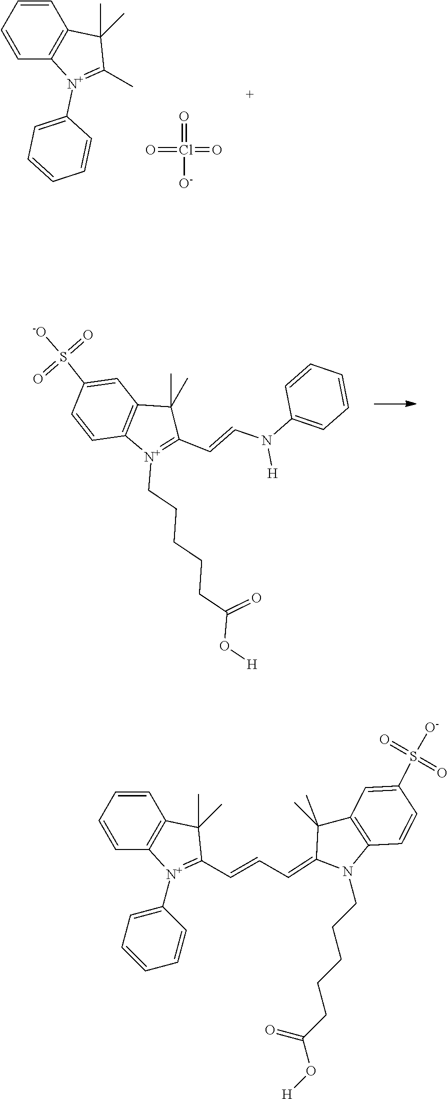

3,3-Dimethyl-1-(5-carboxypenthyl-2-(4-anilinovinyl)-3H-indolium-5-sulfonate (0.46 g) and 2,3,3-Trimethyl-1-phenyl-3H-indolium perchlorate (0.34 g) in mixture of acetic anhydride (2 ml) and acetic acid (1 ml) were stirred at room temperature (˜25° C.) for 0.5 hour. Then to this solution pyridine (0.5 ml) was added. The reaction mixture was stirred at 80° C. for 3 h. Completion of the reaction was checked by TLC (20% H₂O in CH₃CN) and by UV measurement. Once the reaction finished, the red coloured mixture was cooled down and the solvents were removed under vacuum. The residue was purified by C18 flash column (TEAB 0.1 M in water and acetonitrile). Yield: 0.33 g (55%).

### Example 48: Compound XXIV Indocarbocyanine

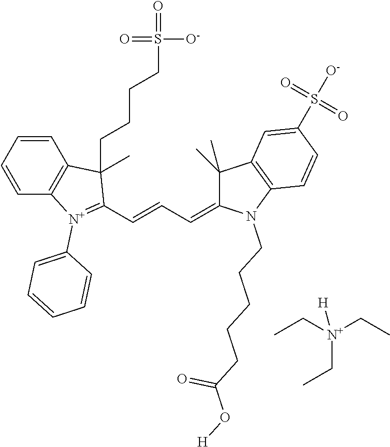

Chemical Name: Triethylammonium 2-{(5-[(4-sulfonatobutyl)-1-phenyl-3-methyl)-1,2-dihydro-3H-indol-2-ylidene]-1-propen-1-yl}-3,3-dimethyl-1-(5-carboxypenthyl)-indolium-5-sulfonate.

Reaction Scheme:

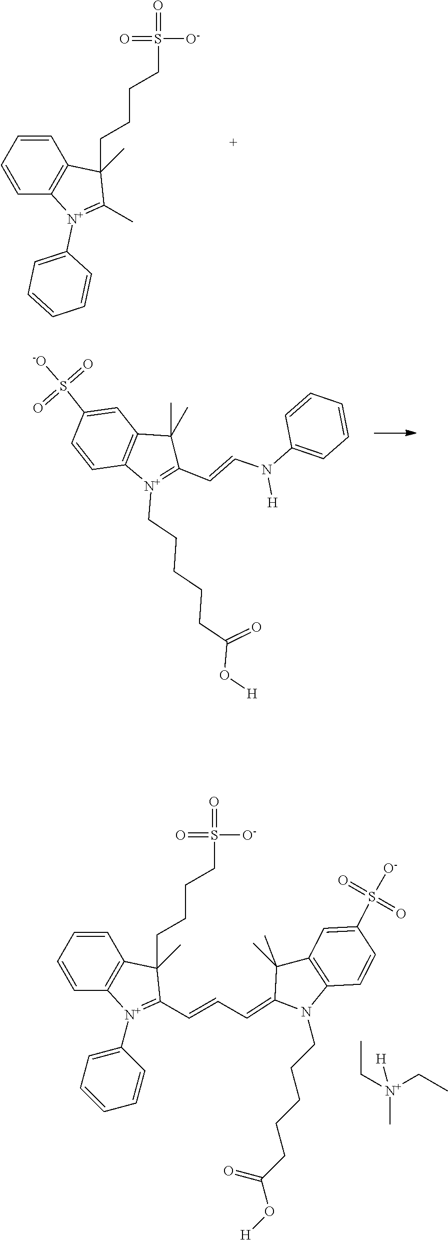

3,3-Dimethyl-1-(5-carboxypenthyl-2-(4-anilinovinyl)-3H-indolium-5-sulfonate (0.46 g) and 2,3-dimethyl-3-(4-sulfonatobutyl)-1-phenyl-3H-indolium (0.36 g) in mixture of acetic anhydride (2 ml) and acetic acid (1 ml) were stirred at room temperature (˜25° C.) for 0.5 hour. Then to this solution pyridine (1 ml) was added. The reaction mixture was stirred at 80° C. for 3 h/completion of the reaction checked by TLC (20% H₂O in CH₃CN)/and by UV measurement). Once the reaction finished, the red coloured reaction mixture was cooled down and most of the solvents were removed under vacuum. The residue was purified by C18 flash column (TEAB 0.1 M in water and acetonitrile). Yield: 0.29 g (35%).

### Example 49: Compound XXV Indocarbocyanine

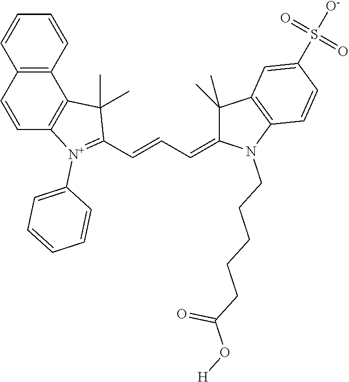 [SMILES: CC1(C)C(/C=C/C=C2/N(CCCCCC(=O)O)c3ccc(S(=O)(=O)[O-])cc3C2(C)C)=[N+](c2ccccc2)c2ccc3ccccc3c21]

Chemical Name: 2-{(5-[(3-phenyl-1,1-dimethyl)-2,3-dihydro-1H-benzo[e]indol-2-ylidene]-1-propen-1-yl}-3,3-dimethyl-1-(5-carboxypenthyl)-indolium-5-sulfonate.

Reaction Scheme:

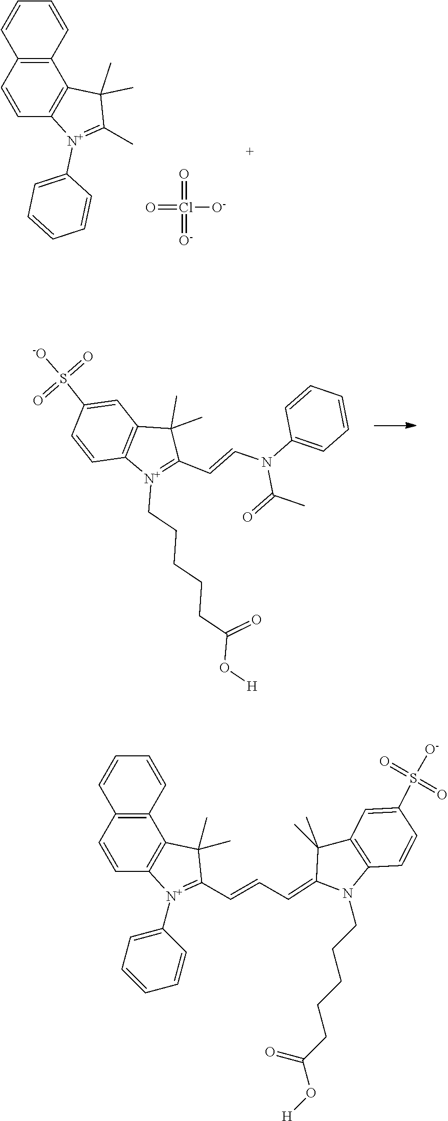

3,3-Dimethyl-1-(5-carboxypenthyl-2-(4-anilidovinyl)-3H-indolium-5-sulfonate (0.46 g) and 1,1,2-trimethyl-3-phenyl-3H-indolium perchlorate (0.39 g) in mixture of acetic anhydride (1 ml) and acetic acid (1 ml) were stirred at room temperature (˜25° C.) for 0.5 hour. Then to this solution pyridine (1 ml) was added. The reaction mixture was stirred at 60° C. for 3 h/the reaction progress checked by TLC (20% H₂O in CH₃CN)/and by UV measurement. Once the reaction finished, the red coloured reaction mixture was cooled down and most of the solvents were removed under vacuum. The residue was purified by C18 flash column (TEAB 0.1 M in water and acetonitrile). Yield: 0.38 g (54%).

### Example 50: Compound XXVI Dye conjugate pppT-I-2

Reaction Scheme:

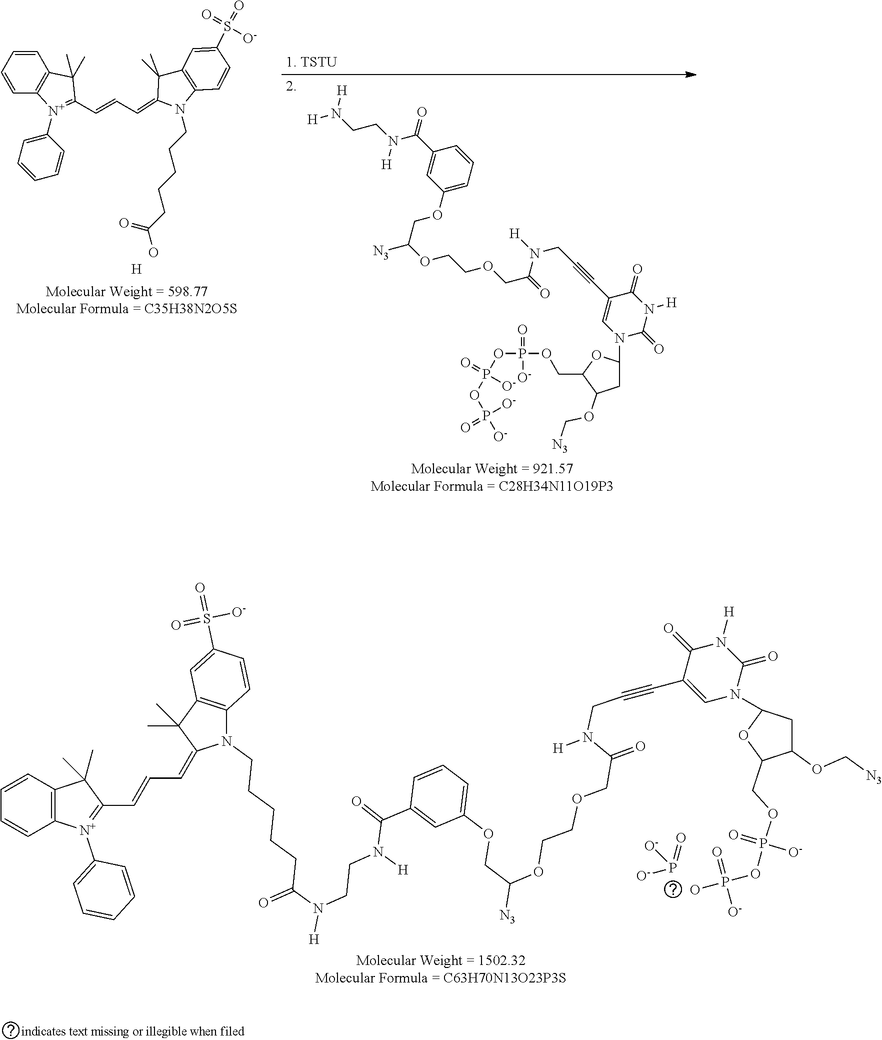

Preparation: Anhydrous DMA (5 mL) and Hunig's Base (0.06 mL) were added to the dried sample of the dye (Compound XXIII) (60 mg). A solution of TSTU, (0.25 g) in 5 mL of dry DMA was then added to this. The red colour of activated ester developed. The reaction mixture was stirred at room temperature for 1 h. According to TLC (20% H₂O in CH₃CN) the activation was completed. After activation was completed this solution was added to the solution of pppT-LN3 (0.23 g) in water (7 mL). The reaction mixture was stirred at room temperature under nitrogen atmosphere for 3 h. The coupling progress was checked by TLC (20% H₂O in acetonitrile). The reaction mixture was cooled down to ˜4° C. with an ice-bath, then a solution of 0.1 M TEAB (5 mL) in water was added and the mixture was stirred at room temperature for 10 min. The reaction mixture was applied to column with ˜ 50 g of DEAE sephadex resin suspension in 0.05 M TEAB solution in water and washed with TEAB (concentration gradient from 0.1 M up to 0.5 M). Coloured fractions were collected and evaporated then co-evaporated again with water to remove more TEAB and vac down to dryness. The residue was then re-dissolved in TEAB 0.1 M. This solution was filtered through a syringe filter 0.2 nm pore size into a corning flask and stored in the freezer. The product was purified by HPLC using C18 reverse phase column with acetonitrile-0.1 M TEAB. Yield 67%.

### Example 51: Compound XXVII Dye Conjugate pppT-I-4

Reaction Scheme:

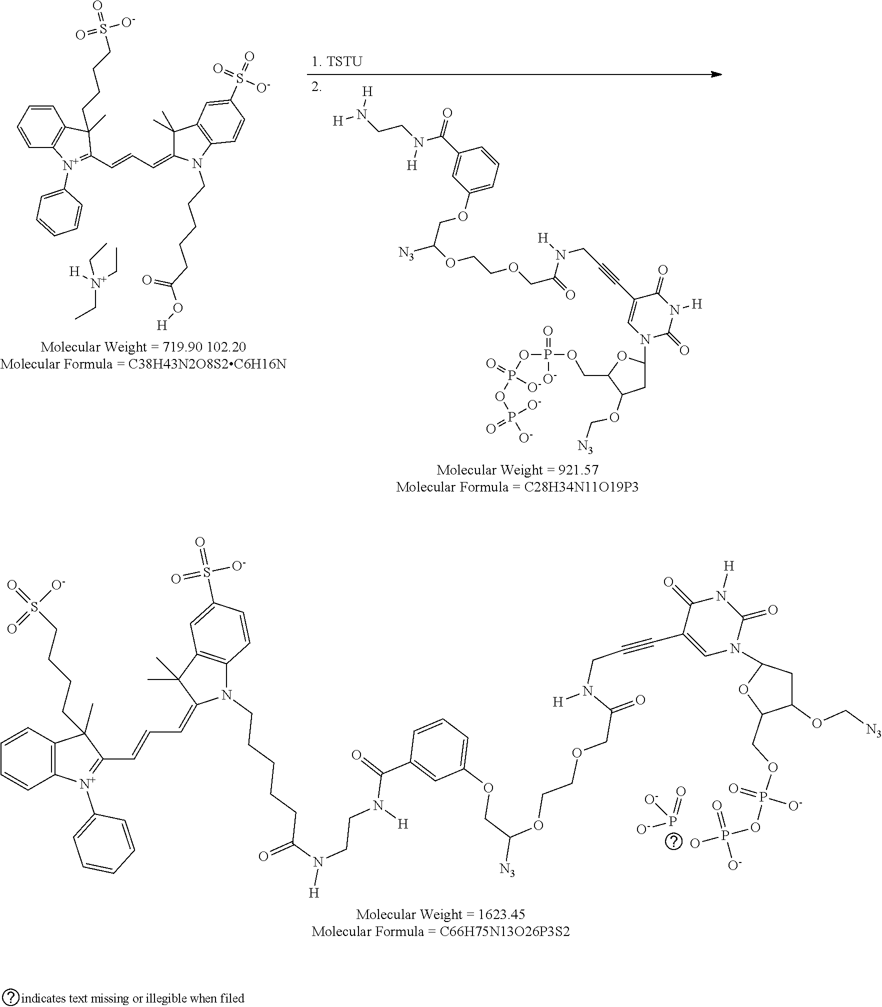

Preparation: Anhydrous DMA (5 mL) and Hunig's Base (0.06 mL) were added to the dried sample of the dye (Compound XXIII) (82 mg). A solution of TSTU, (0.25 g) in 5 mL of dry DMA was then added to this. The red colour of activated ester developed soon. The reaction mixture was stirred at room temperature for 1 h. After activation was completed (TLC: 15% H₂O in CH₃CN) this solution was added to the solution of pppT-LN3 (0.23 g) in water (7 mL). The reaction mixture was stirred at room temperature under nitrogen atmosphere for 3 h. The reaction mixture was cooled down to ˜4° C. with an ice-bath, then a solution of 0.1 M TEAB (5 mL) in water was added and the mixture was stirred at room temperature for 10 min. The reaction mixture was applied to column with ˜75 g of DEAE Sephadex resin suspension in 0.05 M TEAB solution in water and washed with TEAB (concentration gradient from 0.10 M up to 0.75 M). Red coloured fractions were collected, the solvent evaporated and then the residue co-evaporated again with water to remove more TEAB and vac down to dryness. The dye was then re-dissolved in TEAB 0.1 M. This solution was filtered through a syringe filter 0.2 nm pore size and the product was purified by HPLC using C18 reverse phase column with acetonitrile-0.1 M TEAB. Yield 70%.

The terms “substantially” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Also, when used herein, an indefinite article such as “a” or “an” means “at least one.”

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

A number of implementations have been described. Nevertheless, it will be understood that various modifications may be made without departing from the spirit and scope of the specification.

In addition, the logic flows depicted in the figures do not require the particular order shown, or sequential order, to achieve desirable results. In addition, other processes may be provided, or processes may be eliminated, from the described flows, and other components may be added to, or removed from, the described systems. Accordingly, other implementations are within the scope of the following claims.

While certain features of the described implementations have been illustrated as described herein, many modifications, substitutions, changes and equivalents will now occur to those skilled in the art. It is, therefore, to be understood that appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

1. A method, comprising:
providing a sample, the sample including a first nucleotide and a second nucleotide;
contacting the sample with a first fluorescent dye and a second fluorescent dye, the first fluorescent dye emitting first emitted light within a first wavelength band responsive to a first excitation illumination light, the second fluorescent dye emitting second emitted light within a second wavelength band responsive to a second excitation illumination light;
simultaneously collecting, using one or more image detectors, multiplexed fluorescent light comprising the first emitted light and the second emitted light, the first emitted light being a first color channel corresponding to the first wavelength band and the second emitted light being a second color channel corresponding to the second wavelength band; and
identifying the first nucleotide based on the first wavelength band of the first color channel and the second nucleotide based on the second wavelength band of the second color channel.

2. The method as in claim 1, wherein the first wavelength band corresponds to a blue color and the second wavelength band corresponds to a green color.

3. The method as in claim 1, wherein the first wavelength band is included within a range of about 450 nm to about 525 nm, and wherein the second wavelength band is included within a range of about 525 nm to about 650 nm.

4. The method as in claim 1, wherein a first mean or peak wavelength is defined for a first emission spectrum of the first fluorescent dye, and a second mean or peak wavelength is defined for a second emission spectrum of the second fluorescent dye, the first and second mean or peak wavelengths having at least a predefined separation from each other.

5. The method as in claim 1, wherein the first wavelength band has shorter wavelengths than the second wavelength band, wherein the second wavelength band is associated with a first wavelength, and wherein a wavelength emission separation between the first fluorescent dye and the second fluorescent dye is defined so that an emission spectrum of the first fluorescent dye includes at most a predefined amount of light at or above the first wavelength.

6. The method as in claim 1, wherein simultaneously collecting the multiplexed fluorescent light includes:
detecting the first emitted light using a first optical subsystem for the first color channel, and
detecting the second emitted light using a second optical subsystem for the second color channel,
wherein an emission dichroic filter directs the first emitted light of the first color channel to the first optical subsystem and the second emitted light of the second color channel to the second optical subsystem.

7. The method as in claim 6, wherein at least one of the first optical subsystem and the second optical subsystem includes an angled optical path.

8. The method as in claim 1, wherein an emission spectrum of the first fluorescent dye has a peak in the first wavelength band.

9. The method as in claim 1, wherein the sample further includes a third nucleotide, and
wherein the method further comprises:
contacting the sample with a third fluorescent dye emitting third emitted light within the first wavelength band responsive to the first excitation illumination light, and emitting fourth emitted light within the second wavelength band responsive to the second excitation illumination light, wherein the multiplexed fluorescent light further comprises the third emitted light and the fourth emitted light; and
identifying the third nucleotide based on the first wavelength band of the first color channel and on the second wavelength band of the second color channel.

10. The method as in claim 1, wherein the sample further includes a third nucleotide, and
wherein the method further comprises:
contacting the sample with a third fluorescent dye emitting third emitted light within a third wavelength band responsive to a third excitation illumination light, wherein the multiplexed fluorescent light further comprises the third emitted light; and
identifying the third nucleotide based on the third wavelength band.

11. An apparatus, comprising:
a flow cell containing a sample, the sample including a first nucleotide and a second nucleotide, wherein the first nucleotide is coupled to a first fluorescent dye, wherein the second nucleotide is coupled to a second fluorescent dye, the first fluorescent dye emitting first emitted light within a first wavelength band responsive to a first excitation illumination light, the second fluorescent dye emitting second emitted light within a second wavelength band responsive to a second excitation illumination light;
an illumination system simultaneously providing the first excitation illumination light and the second excitation illumination light to the flow cell; and
a light collection system simultaneously collecting multiplexed fluorescent light comprising the first emitted light and the second emitted light, the first emitted light being a first color channel corresponding to the first wavelength band and the second emitted light being a second color channel corresponding to the second wavelength band.

12. The apparatus as in claim 11, wherein the first wavelength band corresponds to a blue color and the second wavelength band corresponds to a green color.

13. The apparatus as in claim 11, wherein the first wavelength band is included within a range of about 450 nm to about 525 nm, and wherein the second wavelength band is included within a range of about 525 nm to about 650 nm.

14. The apparatus as in claim 11, wherein a first mean or peak wavelength is defined for a first emission spectrum of the first fluorescent dye, and a second mean or peak wavelength is defined for a second emission spectrum of the second fluorescent dye, the first and second mean or peak wavelengths having at least a predefined separation from each other.

15. The apparatus as in claim 11, wherein the first wavelength band has shorter wavelengths than the second wavelength band, wherein the second wavelength band is associated with a first wavelength, and wherein a wavelength emission separation between the first fluorescent dye and the second fluorescent dye is defined so that an emission spectrum of the first fluorescent dye includes at most a predefined amount of light at or above the first wavelength.

16. The apparatus as in claim 11, wherein the light collection system includes:
a first optical subsystem for the first color channel detecting the first emitted light, and
a second optical subsystem for the second color channel detecting the second emitted light,
wherein an emission dichroic filter directs the first emitted light of the first color channel to the first optical subsystem and the second emitted light of the second color channel to the second optical subsystem.

17. The apparatus as in claim 16, wherein at least one of the first optical subsystem and the second optical subsystem includes an angled optical path.

18. The apparatus as in claim 11, wherein an emission spectrum of the first fluorescent dye has a peak in the first wavelength band.

19. The apparatus as in claim 11, wherein the sample further includes a third nucleotide coupled to a third fluorescent dye emitting third emitted light within the first wavelength band responsive to the first excitation illumination light, and emitting fourth emitted light within the second wavelength band responsive to the second excitation illumination light, and wherein the multiplexed fluorescent light further comprises the third emitted light and the fourth emitted light.

20. The apparatus as in claim 11, wherein the sample further includes a third nucleotide coupled to a third fluorescent dye emitting third emitted light within a third wavelength band responsive to a third excitation illumination light, wherein the multiplexed fluorescent light further comprises the third emitted light.
